(19) United States
(12) Patent Application Publication
(10) Pub. No.: US 2023/0192789 A1
(43) Pub. Date: Jun. 22, 2023

(54) STRUCTURALLY-STABILIZED AND HDMX-SELECTIVE P53 PEPTIDES AND USES THEREOF

(71) Applicant: Dana-Farber Cancer Institute, Inc., Boston, MA (US)

(72) Inventors: Loren D. Walensky, Newton, MA (US); Yael Ben-Nun, Ramat Gan (IL); Henry D. Herce, Brookline, MA (US)

(21) Appl. No.: 17/920,503

(22) PCT Filed: Apr. 27, 2021
(86) PCT No.: PCT/US2021/029391

§ 371 Date: Oct. 21, 2022

Related U.S. Application Data
(60) Provisional application No. 63/016,130, filed on Apr. 27, 2020.

(51) Int. Cl. C07K 14/47; G01N 33/50; A61P 35/00

(52) U.S. Cl. CPC C07K 14/4747; A61P 35/00; G01N 33/5011; A61K 38/00

(57) ABSTRACT

Disclosed herein are peptides and structurally-stabilized peptides that selectively bind to HDMX, or both HDMX and HDM2 as well as compositions comprising the same. Also provided are methods for using such peptides in the treatment and diagnosis of cancer (e.g., HDMX-expressing and/or -dependent cancers).

### CROSS-REFERENCE TO RELATED APPLICATION

This application claims the priority benefit of U.S. Provisional Application No. 63/016,130, filed Apr. 27, 2020, which is hereby incorporated by reference herein in its entirety.

### SEQUENCE LISTING

The instant application contains a Sequence Listing which has been submitted electronically in ASCII format and is hereby incorporated by reference in its entirety. Said ASCII copy, created on Apr. 26, 2021, is named 00530-0355WO1_SL.txt and is 116,335 bytes in size.

### TECHNICAL FIELD

This disclosure relates to HDMX-selective peptides based on the transactivation domain of p53, structurally-stabilized peptides thereof, compositions comprising the same, methods of making the same, and methods for using such peptides in the treatment and diagnosis of cancer.

### BACKGROUND

As the “guardian of the genome,” p53 prevents the emergence of genetically variant clones by activating defense mechanisms, such as induction of senescent-like arrest and apoptotic programs, to prevent replication of defective cells (Lane, Nature, 358:15-16 (1992); Vogelstein et al., Nature, 408:307-310 (2000)). Cancer cells have adopted a number molecular strategies to subvert p53 activity and achieve a pathologic survival advantage (see, e.g., Baker et al., Science, 244:217-221 (1989); Honda et al., FEBS Lett., 420:25-27 (1997); Momand et al., Cell, 69:1237-1245 (1992); Moll et al., Proc. Natl. Acad. Sci. U.S.A., 92:4407-4411 (1995).

Restoration of p53 activity is a strategy for cancer therapy (see, e.g., Brown et al., Nat. Rev. Cancer, 9:862-873 (2009)). The determination of the crystal structure of the p53-HDM2 binding interface contributed to the development of such strategies, e.g., by revealing that a hydrophobic cleft on the N-terminal surface of the E3 ubiquitin ligase HDM2 (Toledo and Wahl, Nat. Rev. Cancer, 6:909-923 (2006); Marine and Dyer, J. Cell. Sci., 120:371-378 (2007); Bartel et al., Int. J. Cancer, 117:469-475 (2005); Shvarts et al., Genomics, 43 :34-42 (1997); Danovi et al., Mol. Cell. Biol., 24:5835-5843 (2004)) directly engages the amphipathic α-helix of the p53 transactivation domain (Kussie et al., Science, 274:948-953 (1996)). Consequently, small molecules and peptides that target the p53-binding pocket of HDM2 have been developed (see, e.g., Bernal et al., J. Am. Chem. Soc., 129:2456-2457 (2007); Grasberger et al., J. Med. Chem., 48:909-912 (2005); Koblish et al., Mol. Cancer Ther., 5:160-169 (2006); Kritzer et al., J. Am. Chem. Soc., 126:9468-9469 (2004); Shangary et al., Proc. Natl. Acad. Sci., U.S.A., 105:3933-3938 (2008); Vassilev et al., Science, 303:844-848 (2004); Yin et al., Angew. Chem. Int. Ed. Engl., 44:2704-2707 (2005)). One such agent is the small molecule MDM2 inhibitor, Nutlin-3 (Vassilev et al., Science, 303:844-848 (2004)). It has been shown using these agents that targeting HDM2 in certain tumors that express p53 (e.g., wild-type p53) can lead to a therapeutic surge in p53 levels. Specifically, it has been shown that Nutlin-3 can trigger apoptosis in the absence of other therapeutics in certain tumors (see, e.g., Drakos et al., Clin. Cancer Res., 13:3380-3387 (2007); Tabe et al., Clin. Cancer Res., 15:933-942 (2009)). However, such effects do not occur in all tumors types. Specifically, certain tumors are resistant or more resistant to HDM2-targetting therapeutics than other tumors. Co-expression of HDMX, an HDM2 homolog that binds and sequesters p53, with HDM2 can reduce the efficacy of HDM2 targeting agents (see, e.g., Hu et al., J. Biol. Chem., 281:33030-33035 (2006); Patton et al., Cancer Res., 66:3169-3176 (2006); Wade et al., J. Biol. Chem., 281:33036-33044 (2006)).

The role of HDMX in regulating p53 dynamics has been described (see, e.g., Danovi et al., Mol. Cell. Biol., 24:5835-5843 (2004); Laurie et al., Nature, 444:61-66 (2006); Ramos et al., Cancer Res., 61:1839-1842 (2001); Wade et al., J. Biol. Chem., 281:33036-33044 (2006); Wang et al., Proc. Natl. Acad. Sci. U.S.A., 104:12365-12370 (2007)) and in vitro preliminary reports are available for several agents that target HDMX (see, e.g., Harker et al., Bioorg. Med. Chem., 17:2038-2046 (2009); Hayashi et al., Bioorg. Med. Chem., 17:7884-7893 (2009); Hu et al., Cancer Res., 67:8810-8817 (2007); Kallen et al., J. Biol. Chem., 284:8812-8821 (2009); Li et al., J. Am. Chem. Soc., 130:13546-13548 (2008); Michel et al., J. Am. Chem. Soc., 131:6356-6357 (2009); Pazgier et al., Proc. Natl. Acad. Sci. U.S.A., 106:4665-4670 (2009); Reed et al., J. Biol. Chem., 285:10786-10796 (2010)). No such molecules have advanced as clinical candidates to date. Overall, HDMX can cause resistance to HDM2 inhibitors and other therapies by effectively binding to and sequestering p53 protein (Bernal et al., Cancer Cell, 2010). No selective small molecule or stapled peptide inhibitors of HDMX with clinical development potential have been reported to date.

Cancer remains one of the leading causes of morbidity and mortality in the United States. Improved cancer therapies are required.

### SUMMARY

This disclosure relates in part to peptides and stapled peptides designed to selectively target (e.g., inhibit) HDMX (also known as MDMX or MDM4) and thereby block the inhibitory HDMX-p53 interaction. These peptides and stapled peptides selectively (preferentially) bind HDMX over HDM2. The selective stapled peptides disclosed herein are useful for diagnosing and treating cancer and other diseases promoted by HDMX expression and activity.

HDM2 and HDMX are two negative regulators of the tumor suppressor protein p53. Whereas HDM2 binds and destroys p53, HDMX binds and sequesters p53, thereby blocking the anti-cancer activity of p53 and promoting the development, maintenance, and chemoresistance of diverse subtypes of adult and pediatric cancers (Wachter et al., Oncogene, 2017; Stolte et al., J Exp Med, 2018; Howard et al., Cancer Res, 2019).

Selective small molecule inhibitors are available for selective inhibition of HDM2 (e.g., Nutlin-3, RG7388) but have been shown to cause toxicity, particularly hematotoxicity, in clinical trials. Further, HDMX can cause resistance to such molecules by effectively binding to and sequestering p53 protein (Bernal et al., Cancer Cell, 2010). Previously, stapled peptides that target both HDM2 and HDMX (Bernal et al., J Am Chem Soc, 2007; Bernal et al., Cancer Cell, 2010) were developed, and these stapled peptide compounds formed the basis for the development and ongoing Phase 1 and Phase 2 testing of ALRN-6924 (Chang et al., PNAS, 2013; Ng et al., Nat Commun, 2018; Carvajal et al., Sci Transl Med, 2018). However, no selective small molecules with drug-like properties and/or documented on target mechanism of action or stapled peptide inhibitors of HDMX have been reported to date. Certain cancers are exquisitely dependent on HDMX, and thus selective targeting of HDMX can subvert cancer while avoiding the toxicity that accompanies HDM2 targeting, demonstrating the biologically-and clinically-relevant importance of identifying selective HDMX targeting sequences.

The peptides disclosed herein can also be used in diagnostic tests for HDMX dependency and selective HDMX inhibitor susceptibility. For example, if a subject is treated separately with a dual HDMX/HDM2 and a selective HDMX inhibitor, and the selective inhibitor was found to have substantially the same or better activity than the dual inhibitor, this would indicate that the cancer was HDMX-dependent and that treatment could proceed with the selective inhibitor instead of the dual inhibitor. This would allow the subject to receive a more tailored or personalized and potentially less toxic treatment. Thus, the selective stapled peptide inhibitors of HDMX can be used to diagnose an HDMX-expressing or an HDMX-dependent cancer and then used for treatment thereof.

Also provided herein is a crystal structure of selective HDMX inhibitor prototypes in complex with HDMX, revealing the molecular basis for compound selectivity.

It has long been held that the triad of Phe19, Trp23, and Leu26 of wild type p53 is essential for p53 binding to HDM2 and HDMX. Here, it was unexpectedly and surprisingly shown that Leu at position 26 of p53 can be substituted with other amino acids thereby creating a polypeptide that preferentially binds HDMX over HDM2. For instance, mutagenesis scanning analyses performed in the Examples herein revealed that presence of an amino acid other than phenylalanine, isoleucine, norleucine, and leucine (e.g., a charged amino acid (e.g., arginine or glutamic acid), alanine, valine, tryptophan, a hydrophobic amino acid less hydrophobic than leucine (e.g., Ala, Gly, Pro,), or a hydrophilic amino acid) at the amino acid corresponding to position 26 of p53 (numbered according to SEQ ID NO:102) in peptides based on the p53 transactivation domain (e.g., SEQ ID NO:99, SEQ ID NO:100, SEQ ID NO:101) yields an HDMX-selective peptide (i.e., a peptide that preferentially binds to HDMX over HDM2, e.g., has an at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity to HDMX than to HDM2 as determined by, e.g., isothermal calorimetry or fluorescence polarization analyses). Thus, this disclosure features p53 transactivation domain peptides (the transactivation domain refers to amino acids 14-29 of SEQ ID NO:102) and stapled peptides that preferentially bind HDMX relative to HDM2, methods of making such peptides, and methods of using such peptides (e.g., in therapeutic and diagnostic methods). Provided are peptides and stapled peptides based on, or derived from, the transactivation domain of p53 wherein the amino acid corresponding to Leu26 of SEQ ID NO:102 is substituted with any amino acid other than an amino acid more hydrophobic than leucine (excluding tryptophan and valine). Also provided herein are peptides and stapled peptides based on, or derived from, the transactivation domain of p53 wherein the amino acid corresponding to Leu26 of SEQ ID NO:102 is substituted with any amino acid other than phenylalanine, isoleucine, norleucine, and leucine. Such stapled peptides based on, or derived from, the p53 transactivation domain include peptides having the amino acid sequence of positions 14-29 of SEQ ID NO:102 and variants thereof and include stapled peptides having 3-12 (e.g., 3, 4, 5, 6, 7, 8, 9, 10, 11, or 12) amino acid substitutions in and/or 0-5 (e.g., 0, 1, 2, 3, 4, 5) amino acid additions and/or deletions at the N- and/or C-terminus of the amino acid sequence of SEQ ID NO:99, SEQ ID NO:100, or SEQ ID NO:101, wherein two of the 3-12 amino acid substitutions are with stapling amino acids (e.g., α,α-disubstituted non-natural amino acids with olefinic side chains that can be cross-linked (optionally by ring closing metathesis reaction)) and one of the 3-12 amino acid substitutions is at the amino acid corresponding to Leu26 of SEQ ID NO:102 with any amino acid other than phenylalanine, isoleucine, norleucine, and leucine, wherein the peptide preferentially binds HDMX (e.g., has an at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity to HDMX than to HDM2 as determined by, e.g., isothermal calorimetry or fluorescence polarization analyses). In some instances, variants include those identified from phage display. In some instances, the amino acid that is substituted for leucine is a positively (e.g., K, R) or negatively charged (e.g., D, E) amino acid. In some instances, the amino acid that is substituted for leucine is alanine. In some instances, the amino acid that is substituted for leucine is tryptophan. In some instances, the amino acid that is substituted for leucine is valine. In some instances, the amino acid that is substituted for leucine is hydrophilic (e.g., S, T, R, K. N, D, E, Q, H). In some instances, the amino acid that is substituted for leucine is E(OMe) or 5-fluoronorvaline. In some instances, the amino acid that is substituted for leucine is not isoleucine, norleucine, or phenylalanine. In some instances, the amino acid that is substituted for leucine at the position corresponding to position 26 of SEQ ID NO:102 is alanine, arginine, glutamic acid, E(OMe), 5-fluoronorvaline, lysine, glycine, histidine, asparagine, glutamine, serine, threonine, valine, tryptophan, tyrosine, proline, aspartic acid, D(OMe), or cysteine. In some instances, the amino acid that is substituted for leucine at the position corresponding to position 26 of SEQ ID NO:102 is aspartic acid. In some instances, these peptides may further comprise a substitution(s) at the position corresponding to Phe19 and/or Trp23 of SEQ ID NO:102. In some instances, the position corresponding to Phe19 may be substituted with a conservative amino acid substitution. In some instances, the position corresponding to Phe19 may be substituted with alanine. In some instances, the position corresponding to Trp23 may be substituted with a conservative amino acid substitution. In some instances, the position corresponding to Trp23 may be substituted with alanine, arginine, glutamic acid, lysine, or aspartic acid. For example, the position corresponding to Phe19 may be substituted with alanine and/or the position corresponding to Trp23 may be substituted with alanine, arginine, glutamic acid, lysine, or aspartic acid. In some instances, Phe19 is not substituted. In some instances, Trp23 is not substituted. In some instances, one or more of the amino acids corresponding to positions 15, 18, 20, 22, 24, 25, 27, and/or 29 of SEQ ID NO:102 are not substituted. If one or more of the amino acids corresponding to positions 15, 18, 20, 22, 24, 25, 27, and/or 29 of SEQ ID NO:102 are substituted, they are substituted by conservative amino acid substitutions. In some instances, the amino acid corresponding to position 18 of SEQ ID NO:102 is not substituted. In some instances, the amino acids corresponding to positions 18, 19, and 23 of SEQ ID NO:102 are not substituted. In addition, the peptides may comprise 1 to 10 (e.g., 1, 2, 3, 4, 5, 6, 7, 8, 9, 10) substitutions provided that the peptide still preferentially bind HDMX relative to HDM2. Such substitutions may be at the positions corresponding to positions 17, 21, 22, 23, 24, and 29 of SEQ ID NO:102. These peptides may be stabilized - e.g., stapled or stitched. In some instances, a peptide (e.g., stapled or stitched peptide) of this disclosure is a variant of SEQ ID NO:101, SEQ ID NO:99, or SEQ ID NO:100. In certain instances, the peptide differs from the amino acid sequence of any one of SEQ ID NOs:99-101 at 3 to 10 (e.g., 3, 4, 5, 6, 7, 8, 9, 10) positions. Three of those positions include the two amino acids replaced with stapling amino acids for stabilizing the peptide and the amino acid corresponding to Leu26 of SEQ ID NO:102. The two amino acids replaced with stapling amino acids are generally those that are separated by two, three, or six amino acids (see, e.g., FIGS. 3A, 3B, 4A, and 4B). In certain cases, the stapling amino acids are introduced at the positions corresponding to positions 20 and 27 of SEQ ID NO:102. These peptides can be used for treating cancers expressing HDMX or dependent on HDMX. These peptides can also be used for diagnosing cancers expressing HDMX or dependent on HDMX.

The disclosure features HDMX-specific stabilized peptides such as those set forth in SEQ ID NOs: 5, 7, 8, 10, 13, 21, 23, 24, 27, 29, 34, 37, 38, 40, 42-46, 48-51, 56-59, 61, 62, 64, 67-76, 78, 80, 86-94, or 96 or (SEQ ID NO:84)-napthalene-(SEQ ID NO238) as well as HDMX-specific variants thereof. These variants include sequences with 1 to 10 (e.g., 1, 2, 3, 4, 5, 6, 7, 8, 9, 10) substitutions relative to SEQ ID NOs: 5, 7, 8, 10, 13, 21, 23, 24, 27, 29, 34, 37, 38, 40, 42-46, 48-51, 56-59, 61, 62, 64, 67-76, 78, 80, 86-94, or 96, or (SEQ ID NO:84)-napthalene-(SEQ ID NO:238). In some instances, the 1 to 10 (e.g., 1, 2, 3, 4, 5, 6, 7, 8, 9, 10) substitutions are on the non-interacting face of the stabilized peptide helix. In some instances, the 1 to 10 (e.g., 1, 2, 3, 4, 5, 6, 7, 8, 9, 10) substitutions are on the interacting face of the stabilized peptide helix. In some instances, the 1 to 10 (e.g., 1, 2, 3, 4, 5, 6, 7, 8, 9, 10) substitutions are on the non-interacting and interacting faces of the stabilized peptide helix. In some cases, the substitutions are conservative amino acid substitutions. In this context, the “interacting face” refers to the face of the alpha helix that interacts with or binds to HDMX.

Also featured herein is a polypeptide comprising a stabilized p53 peptide that binds HDMX, wherein the stabilized p53 peptide has 3-11 (e.g., 3, 4, 5, 6, 7, 8, 9, 10, 11) amino acid substitutions relative to the sequence of SEQ ID NO:101, 3-13 (e.g., 3, 4, 5, 6, 7, 8, 9, 10, 11, 12, 13) amino acid substitutions relative to the sequence of SEQ ID NO:100, or 3-13 (e.g., 3, 4, 5, 6, 7, 8, 9, 10, 11, 12, 13) amino acid substitutions relative to the sequence of SEQ ID NO:99, wherein at least two of the amino acid substitutions are substitutions of natural amino acids separated by 2, 3, or 6 amino acids with α,α-disubstituted non-natural amino acids with olefinic side chains that can be cross-linked (optionally by ring closing metathesis reaction) and one of the amino acid substitutions is any amino acid other than phenylalanine, isoleucine, norleucine, and leucine (e.g., is glutamic acid, alanine, arginine, valine, or tryptophan) at position 26 (numbered according to SEQ ID NO:102), wherein the peptide has 0-5 additions and/or deletions at the N-terminus and/or C-terminus relative to the sequence of SEQ ID NO:101, SEQ ID NO:100, or SEQ ID NO:99, and wherein the peptide preferentially binds HDMX over HDM2 (e.g., has an at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity to HDMX than to HDM2 as determined by, e.g., isothermal calorimetry or fluorescence polarization analyses). In some instances, the peptide comprises an arginine at the amino acid corresponding to position 22 of SEQ ID NO:102. In some instances, the peptide comprises a Phe at the amino acid corresponding to position 19 of SEQ ID NO:102. In some instances, the peptide comprises a tryptophan at the amino acid corresponding to position 23 of SEQ ID NO:102. In some instances, one or more of the negatively charged residues in a peptide described herein are esterified, optionally by adding OMe to one or more of the negatively charged residues. In some instances, the peptide comprises the amino acid sequence of any one of SEQ ID NO:13, 23, 27, 40, 42, 45, 58, 51, 56-59, 61, 62, 64, 67-75, 78, 80, 82-94, and 96-98. Also provided herein are methods of making the polypeptides, compositions comprising the polypeptides, and methods of using the polypeptides (e.g., to treat or diagnose a cancer (e.g., an HDMX-expressing or HDMX-dependent cancer).

The disclosure also features a pharmaceutical composition comprising (a) means for preferentially binding HDMX over HDM2; and (b) a pharmaceutically acceptable carrier.

The disclosure also features HDM2-binding stabilized peptides such as the one set forth in SEQ ID NO: 47, as well as HDM2-binding variants thereof. These variants include sequences with 1 to 10 (e.g., 1, 2, 3, 4, 5, 6, 7, 8, 9, 10) substitutions relative to SEQ ID NO: 47.

The disclosure also features a pharmaceutical composition comprising (a) means for preferentially binding HDM2 over HDMX; and (b) a pharmaceutically acceptable carrier.

Also featured are HDM2/HDMX dual binding stabilized peptides such as those set forth in SEQ ID NOs: 2-4, 6, 9, 11, 12, 14-20, 22, 25, 26, 28, 30, 31-33, 35, 36, 39, 41, 47, 60, 63, 65, 66, 77, 79, and 81, as well as variants thereof. Variants include sequences with 1 to 10 (e.g., 1, 2, 3, 4, 5, 6, 7, 8, 9, 10) substitutions relative to SEQ ID NOs: 2-4, 6, 9, 11, 12, 14-20, 22, 25, 26, 28, 30, 31-33, 35, 36, 39, 41, 47, 60, 63, 65, 66, 77, 79, and 81. These variants bind both HDM2 and HDMX. In some instances, the HDM2/HDMX dual binding stabilized peptide comprises the amino acid sequence set forth in one of SEQ ID NOs: 9, 22, 26, 47, 60, 77, 79, and 81 and variants thereof. Variants include sequences with 1 to 10 (e.g., 1, 2, 3, 4, 5, 6, 7, 8, 9, 10) substitutions relative to SEQ ID NOs: 9, 22, 26, 47, 60, 77, 79, and 81. These variants bind both HDM2 and HDMX.

The disclosure also features a pharmaceutical composition comprising (a) means for selectively binding HDM2 and HDMX; and (b) a pharmaceutically acceptable carrier.

In another aspect, this disclosure features a structurally-stabilized peptide that preferentially binds to HDMX over HDM2, wherein the structurally -stabilized peptide comprises the amino acid sequence LTFEEYWAQBTSAA (SEQ ID NO:101), with 3 to 10 amino acid substitutions relative to the amino acid sequence of SEQ ID NO:101, wherein at least two of the 3 to 10 amino acid substitutions are substitutions with stapling amino acids, wherein one of the 3 to 10 amino acid substitutions is a substitution of the amino acid corresponding to L26 of SEQ ID NO:102 with a charged amino acid, alanine, or a hydrophilic amino acid, and optionally wherein the 3 to 10 amino acid substitutions comprises (i) substitution of the amino acid corresponding to F19 of SEQ ID NO:102 with a charged amino acid or alanine, or (ii) substitution of the amino acid corresponding to W23 of SEQ ID NO:102 with a charged amino acid, alanine, or an aromatic amino acid, wherein the structurally-stabilized peptide is stapled or stitched. In some instances, the structurally-stabilized peptide disclosed herein includes additional amino acids (e.g., at least 1, 2, 3, 4, 5, 6, 7, 8, 9, or 10) at the N-terminus of SEQ ID NO:101. In some instances, the structurally-stabilized peptide disclosed herein includes additional amino acids (e.g., at least 1, 2, 3, 4, 5, 6, 7, 8, 9, or 10) at the C-terminus of SEQ ID NO:101. In some instances, the structurally-stabilized peptide disclosed herein includes 0-5 (e.g., 0, 1, 2, 3, 4, 5) amino acid deletions at the N-terminus of SEQ ID NO:101. In some instances, the structurally-stabilized peptide disclosed herein includes 0-5 (e.g., 0, 1, 2, 3, 4, 5) amino acid deletions at the C-terminus of SEQ ID NO:101.

In another aspect, this disclosure features a structurally-stabilized peptide that preferentially binds to HDMX over HDM2, wherein the structurally -stabilized peptide comprises the amino acid sequence LSQETFSDLWKLLPEN (SEQ ID NO:99), with 3 to 10 amino acid substitutions relative to the amino acid sequence of SEQ ID NO:99, wherein at least two of the 3 to 10 amino acid substitutions are substitutions with stapling amino acids, wherein one of the 3 to 10 amino acid substitutions is a substitution of the amino acid corresponding to L26 of SEQ ID NO:102 with a charged amino acid, alanine, or a hydrophilic amino acid, and optionally wherein the 3 to 10 amino acid substitutions comprises: (i) substitution of the amino acid corresponding to F19 of SEQ ID NO:102 with a charged amino acid or alanine, or (ii) substitution of the amino acid corresponding to W23 of SEQ ID NO:102 with a charged amino acid, alanine, or an aromatic amino acid, wherein the structurally-stabilized peptide is stapled or stitched. In some instances, the structurally-stabilized peptide disclosed herein includes additional amino acids (e.g., at least 1, 2, 3, 4, 5, 6, 7, 8, 9, or 10) at the N-terminus of SEQ ID NO: 99. In some instances, the structurally-stabilized peptide disclosed herein includes additional amino acids (e.g., at least 1, 2, 3, 4, 5, 6, 7, 8, 9, or 10) at the C-terminus of SEQ ID NO:99. In some instances, the structurally-stabilized peptide disclosed herein includes 0-5 (e.g., 0, 1, 2, 3, 4, 5) amino acid deletions at the N-terminus of SEQ ID NO:99. In some instances, the structurally-stabilized peptide disclosed herein includes 0-5 (e.g., 0, 1, 2, 3, 4, 5) amino acid deletions at the C-terminus of SEQ ID NO:99.

In another aspect, this disclosure features a structurally-stabilized peptide that preferentially binds to HDMX over HDM2, wherein the structurally-stabilized peptide comprises the amino acid sequence QSQQTFSNLWRLLPQN (SEQ ID NO:100), with 3 to 10 amino acid substitutions relative to the amino acid sequence of SEQ ID NO:100, wherein at least two of the 3 to 10 amino acid substitutions are substitutions with stapling amino acids, wherein one of the 3 to 10 amino acid substitutions is a substitution of the amino acid corresponding to L26 of SEQ ID NO:102 with a charged amino acid, alanine, or a hydrophilic amino acid, and optionally wherein the 3 to 10 amino acid substitutions comprises: (i) substitution of the amino acid corresponding to F19 of SEQ ID NO:102 with a charged amino acid or alanine, or (ii) substitution of the amino acid corresponding to W23 of SEQ ID NO:102 with a charged amino acid, alanine, or an aromatic amino acid, wherein the structurally-stabilized peptide is stapled or stitched. In some instances, the structurally-stabilized peptide disclosed herein includes additional amino acids (e.g., at least 1, 2, 3, 4, 5, 6, 7, 8, 9, or 10) at the N-terminus of SEQ ID NO:100. In some instances, the structurally-stabilized peptide disclosed herein includes additional amino acids (e.g., at least 1, 2, 3, 4, 5, 6, 7, 8, 9, or 10) at the C-terminus of SEQ ID NO:100. In some instances, the structurally-stabilized peptide disclosed herein includes 0-5 (e.g., 0, 1, 2, 3, 4, 5) amino acid deletions at the N-terminus of SEQ ID NO:100. In some instances, the structurally-stabilized peptide disclosed herein includes 0-5 (e.g., 0, 1, 2, 3, 4, 5) amino acid deletions at the C-terminus of SEQ ID NO:100.

In certain embodiments, one of the 2 to 10 amino acid substitutions in the structurally-stabilized peptide disclosed herein is a substitution of the amino acid corresponding to L26 of SEQ ID NO:102 with a charged amino acid or alanine.

This disclosure also features a structurally-stabilized peptide that preferentially binds to HDMX over HDM2, wherein the structurally-stabilized peptide includes the amino acid sequence LTF8EYWAQBXSAA, wherein 8 is a first stapling amino acid, X is a second stapling amino acid, and B is cyclobutyl alanine (SEQ ID NO:55), with 1 to 10 amino acid substitutions relative to the amino acid sequence of SEQ ID NO:55, wherein the 1 to 10 amino acid substitutions are not at positions 4 or 11 of SEQ ID NO:55, wherein at least one of the amino acid substitutions is at the amino acid position corresponding to Q16, E17, F19, D21, L22, W23, K24, L26, or N29, or combinations thereof, of the amino acid sequence of SEQ ID NO:102, and wherein a sidechain of the first stapling amino acid is cross-linked to a sidechain of the second stapling amino acid. In certain embodiments, the structurally-stabilized peptide that preferentially binds to HDMX over HDM2, wherein the structurally-stabilized peptide includes the amino acid sequence LSQETF8DLWKLLXEN, wherein 8 is a first stapling amino acid and X is a second stapling amino acid (SEQ ID NO:1), with 1 to 10 amino acid substitutions relative to the amino acid sequence of SEQ ID NO:1, wherein the 1 to 10 amino acid substitutions are not at positions 7 or 14 of SEQ ID NO:1, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid. In certain embodiments, the structurally-stabilized peptide that preferentially binds to HDMX over HDM2, wherein the structurally-stabilized peptide includes the amino acid sequence QSQQTF8NLWRLLXQN, wherein 8 is a first stapling amino acid and X is a second stapling amino acid (SEQ ID NO:95), with 1 to 10 amino acid substitutions relative to the amino acid sequence of SEQ ID NO:95, wherein the 1 to 10 amino acid substitutions are not at positions 7 or 14 of SEQ ID NO:95, wherein at least one of the amino acid substitutions is at the amino acid position corresponding to Q16, E17, F19, D21, L22, W23, K24, L26, or N29, or combinations thereof, of the amino acid sequence of SEQ ID NO:102, and wherein a sidechain of the first stapling amino acid is cross-linked to a sidechain of the second stapling amino acid. In some instances, the structurally-stabilized peptide disclosed herein includes additional amino acids (e.g., at least 1, 2, 3, 4, 5, 6, 7, 8, 9, or 10) at the N-terminus. In some instances, the structurally-stabilized peptide disclosed herein includes additional amino acids (e.g., at least 1, 2, 3, 4, 5, 6, 7, 8, 9, or 10) at the C-terminus.

In certain embodiments, the structurally-stabilized peptide preferentially binds to HDMX with at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity than a binding affinity for HDM2. In some instances, a binding affinity is determined using methods known in the art or described herein, e.g., isothermal calorimetry or fluorescence polarization analyses.

In certain embodiments, the structurally-stabilized peptide includes the amino acid sequence LTF8EYWAQBXSAA, wherein 8 is a first stapling amino acid, X is a second stapling amino acid, and B is cyclobutyl alanine (SEQ ID NO:55), with 1 to 10 amino acid substitutions relative to the amino acid sequence of SEQ ID NO:55, wherein the 1 to 10 amino acid substitutions are not at positions 4 or 11 of SEQ ID NO:55, wherein at least one of the amino acid substitutions is at the amino acid position corresponding to Q16, E17, F19, L22, W23, K24, L26, or N29, or combinations thereof, of the amino acid sequence of SEQ ID NO:102, and wherein a sidechain of the first stapling amino acid is cross-linked to a sidechain of the second stapling amino acid.

In certain embodiments, the structurally-stabilized peptide includesthe amino acid sequence LSQETF8DLWKLLXEN, wherein 8 is a first stapling amino acid and X is a second stapling amino acid (SEQ ID NO:1), with 1 to 10 amino acid substitutions relative to the amino acid sequence of SEQ ID NO:1, wherein the 1 to 10 amino acid substitutions are not at positions 7 or 14 of SEQ ID NO:1, wherein at least one of the amino acid substitutions is at the amino acid position corresponding to Q16, E17, F19, D21, L22, W23, K24, L26, or N29, or combinations thereof, of the amino acid sequence of SEQ ID NO:102, and wherein a sidechain of the first stapling amino acid is cross-linked to a sidechain of the second stapling amino acid.

In certain embodiments, the structurally-stabilized peptide includes the amino acid sequence QSQQTF8NLWRLLXQN, wherein 8 is a first stapling amino acid and X is a second stapling amino acid (SEQ ID NO:95), with 1 to 10 amino acid substitutions relative to the amino acid sequence of SEQ ID NO:95, wherein the 1 to 10 amino acid substitutions are not at positions 7 or 14 of SEQ ID NO:95, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid.

In certain embodiments, the structurally-stabilized peptide includes amino acid substitutions at particular positions. For instance, in certain embodiments, the structurally-stabilized peptide includes a substitution at the amino acid position corresponding to E17 of the amino acid sequence of SEQ ID NO:102 is with a negatively charged amino acid. In certain embodiments, the structurally-stabilized peptide includes a substitution at the amino acid position corresponding to F19 of the amino acid sequence of SEQ ID NO:102 is with a charged amino acid or alanine. In certain embodiments, the structurally-stabilized peptide includes a substitution at the amino acid position corresponding to L22 of the amino acid sequence of SEQ ID NO:102 is with a positively charged amino acid. In certain embodiments, the structurally-stabilized peptide includes a substitution at the amino acid position corresponding to W23 of the amino acid sequence of SEQ ID NO:102 is with a charged amino acid, alanine, or an aromatic amino acid. In certain embodiments, the structurally-stabilized peptide includes a substitution at the amino acid position corresponding to K24 of the amino acid sequence of SEQ ID NO:102 is with a charged amino acid or an aromatic amino acid. In certain embodiments, the structurally-stabilized peptide includes a substitution at the amino acid position corresponding to L26 of the amino acid sequence of SEQ ID NO:102 is with any amino acid other than phenylalanine, isoleucine, norleucine, and leucine. In certain embodiments, the structurally-stabilized peptide includes a substitution at the amino acid position corresponding to L26 of the amino acid sequence of SEQ ID NO:102 is with a charged amino acid, alanine, a hydrophobic amino acid less hydrophobic than leucine (e.g., Ala, Gly, Pro), or a hydrophilic amino acid. In certain embodiments, the structurally-stabilized peptide includes a substitution at the amino acid position corresponding to L26 of the amino acid sequence of SEQ ID NO:102 is with valine or tryptophan. In certain embodiments, the structurally-stabilized peptide includes a substitution at the amino acid position corresponding to N29 of the amino acid sequence of SEQ ID NO:102 is with a positively charged amino acid or an aromatic amino acid.

In certain embodiments, at least one of the amino acid substitutions is at the amino acid position corresponding to F19, L22, W23, K24, L26, and N29, or combinations thereof. In certain embodiments, at least one of the amino acid substitutions is at the amino acid position corresponding to L22, W23, K24, L26, and N29, or combinations thereof. In certain embodiments, the substitution at position F19 is with a charged amino acid or alanine. In certain embodiments, the substitution at position L22 is with a positively charged amino acid. In certain embodiments, the substitution at position W23 is with an aromatic amino acid. In certain embodiments, the substitution at position K24 is with a hydrophobic or positively charged amino acid. In certain embodiments, the substitution at position L26 is with a charged amino acid or alanine. In certain embodiments, the substitution at position N29 is with a hydrophobic amino acid.

In certain embodiments, the substitution at position L22 is with arginine (R), the substitution at position W23 is with naphthalene, the substitution at position K24 is with arginine (R), leucine (L), or phenylalanine (F), the substitution at position L26 is with alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C), and the substitution at position N29 is with leucine (L) or phenylalanine (F).

In certain embodiments, the 1 to 10 amino acid substitutions comprise one or more of L26A; L26R; L26E; L26h, wherein h is homoglutamic acid; L26f, wherein f is 5-fluoronorvaline (f); L26E(OMe), wherein E(OMe) is O-methylated glutamic acid; L26G; L26H; L26K; L26N; L26Q; L26S; L26T; L26V; L26W; L26Y; L26P; L26C; L26D; L22R; W23Z and L26E, wherein Z is naphthalene; L22R and L26E; K24R and L26E; K24L and L26E; K24F and L26E; N29L and L26E; N29F and L26E; D21L and L26E; D21F and L26E; or L26Met(O), wherein Met(O) is methionine-sulfoxide.

In certain embodiments, the structurally-stabilized peptide is 14 to 50 amino acids in length.

In certain embodiments, the at least one of the amino acid substitutions of the structurally-stabilized peptide is at the amino acid position corresponding to F19, L22, W23, K24, L26, and N29, or combinations thereof, wherein the substitution at position F19 is with a charged amino acid or alanine, the substitution at position L22 is with a positively charged amino acid, the substitution at position W23 is with a charged amino acid or alanine, the substitution at position K24 is with a negatively charged amino acid, the substitution at position L26 is with a charged amino acid or alanine, and the substitution at position N29 is with a positively charged amino acid.

In certain embodiments, the substitution at position F19 is with alanine (A), arginine (R), or glutamate (E), the substitution at position L22 is with arginine (R), the substitution at position W23 is with alanine (A), arginine (R), or glutamate (E), the substitution at position K24 is with glutamate (E), the substitution at position L26 is with alanine (A), arginine (R), or glutamate (E), and the substitution at position N29 is with arginine (R).

In certain embodiments, the 1-8 amino acid substitutions of the structurally-stabilized peptide comprise one or more of F19A; F19R; F19E; L22R; W23A; W23R; W23E; K24E; L26A; L26R; L26E; N29R; K24E and L26R; K24E, L26R, and E28A; K24E and L26A; K24E and L26E; K24E, L26E, and E28A; K24E, L26A and E28A; L26R and E28A; L26A and E28A; or L26E and E28A.

In certain embodiments, the at least one of the amino acid substitutions of the structurally-stabilized peptide is at the amino acid position corresponding to E17 and L26, or combinations thereof, wherein the substitution at position E17 is with a negatively charged amino acid and the substitution at position L26 is with a charged amino acid or alanine.

In certain embodiments, the substitution at position E17 is with O-methylated glutamate (E(OMe)) and substitution at position L26 is with alanine (A), arginine (R), glutamate (E), O-methylated glutamate (E(OMe)), or Met(O) (wherein Met(O) is methionine-sulfoxide). In certain embodiments, the 1 to 10 amino acid substitutions comprise L26E; L26E(OMe); or E17E(OMe) and L26E(OMe). In certain embodiments, the structurally-stabilized peptide is 16 to 50 amino acids in length. In certain embodiments, the 8 of the structurally-stabilized peptide is (R)-α-(7′-octenyl)alanine and X is (S)-α-(4′-pentenyl)alanine, or wherein 8 is (R)-α-(4′-pentenyl)alanine and X is (S)-α-(7′-octenyl)alanine. See FIGS. 3 and 4 for the positions of X and 8.

Also featured herein is a structurally-stabilized peptide comprising an amino acid sequence set forth in any one of SEQ ID NOs:5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), wherein a sidechain of the first stapling amino acid is cross-linked to a sidechain of the second stapling amino acid. In certain embodiments, the structurally-stabilized peptide consists of the amino acid sequence set forth in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), wherein a sidechain of the first stapling amino acid is cross-linked to a sidechain of the second stapling amino acid. In certain embodiments, in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), 8 is (R)-α-(7′-octenyl)alanine and X is (S)-α-(4′-pentenyl)alanine, or 8 is (R)-α-(4′-pentenyl)alanine and X is (S)-α-(7′-octenyl)alanine. See FIGS. 3 and 4 for the positions of X and 8.

Also featured herein is a structurally-stabilized peptide comprising an amino acid sequence set forth in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98 or (SEQ ID NO:84)-napthalene-(SEQ ID NO:238)comprising 1 to 4 amino acid substitutions, wherein the structurally-stabilized peptide is stapled, and wherein the peptide selectively binds to HDMX. In certain embodiments, in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), 8 is (R)-α-(7′-octenyl)alanine and X is (S)-α-(4′-pentenyl)alanine or 8 is (R)-α-(4′-pentenyl)alanine and X is (S)-α-(7′-octenyl)alanine. In certain embodiments, the structurally-stabilized peptide is from 16 to 50 amino acids. See FIGS. 3 and 4 for the positions of X and 8.

Also featured herein is a peptide comprising the amino acid sequence set forth in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98 or (SEQ ID NO:84)-napthalene-(SEQ ID NO:238).

Also featured herein is a structurally-stabilized polypeptide comprising the amino acid sequence set forth in one of SEQ ID NOs: 13, 23, 27, 40, 42, 45, 58, 51, 56-59, 61, 62, 64, 67-75, 78, 80, 82-94, and 96-98.

Also featured herein is a pharmaceutical composition that includes a structurally-stabilized peptide as disclosed herein and a pharmaceutically acceptable carrier.

Also featured herein is a method of making a structurally-stabilized peptide, the method comprising: (a) providing a peptide described herein, and (b) cross-linking the peptide. In some instances, the method comprising: (a) providing a peptide having the sequence set forth in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98 or (SEQ ID NO:84)-napthalene-(SEQ ID NO:238), and (b) cross-linking the peptide. In certain embodiments, in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), 8 is (R)-α-(7′-octenyl)alanine, and wherein X in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), respectively, is (S)-α-(4′-pentenyl)alanine. In certain embodiments, in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238), respectively, X is (R)-α-(4′-pentenyl)alanine, and 8 in any one of SEQ ID NOs: 5, 7, 10, 13, 21, 23, 24, 27, 29, 34, 37, 38, 40, 42-47, 49-51, 57-59, 61, 62, 64, 67-76, 78, 80, 86-94, and 96-98, or (SEQ ID NO:84)-napthalene-(SEQ ID NO:238) is (S)-α-(7′-octenyl)alanine. See FIGS. 3 and 4 for the positions of X and 8.

Also featured herein is a method of treating cancer in a human subject in need thereof, comprising administering to the subject a therapeutically effective amount of any one of the structurally-stabilized peptides disclosed herein. In certain embodiments, the cancer is an HDMX-expressing cancer. In certain embodiments, the cancer is an HDMX-overexpressing or HDMX-dependent cancer. In certain embodiments, the cancer is a hematologic cancer. In certain embodiments, the cancer is a solid cancer. In certain embodiments, the solid cancer is an osteosarcomoa or a choriocarcinoma. In certain embodiments, the cancer is an eosinophilic leukemia. In certain embodiments, the cancer is acute myeloid leukemia. In certain embodiments, the cancer is an osteosarcoma. In certain embodiments, the cancer is a pediatric cancer. In certain embodiments, the pediatric cancer is Ewing sarcoma or a rhabdoid tumor or a diffuse interstitial pontine glioma.

Also featured herein is any one of the structurally-stabilized peptides disclosed herein for use in tailoring a more selective and non-toxic treatment for a patient identified to have an HDMX-dependent cancer.

Also featured herein is a method of selecting a treatment for a cancer in a human subject, the method including obtaining cancer cells from the subject; determining that the cancer cells express HDMX; and administering to the subject a therapeutically effective amount of any one of the structurally-stabilized peptides disclosed herein. In certain embodiments, the methods further includes determining that the structurally-stabilized peptide is cytotoxic to the cancer cells obtained from the human subject in an ex vivo assay.

Also featured herein is a method of selecting a treatment for a cancer in a human subject, the method comprising obtaining cancer cells from the subject; separating the cancer cells into a first sample and a second sample; contacting the cancer cells of the first sample with a HDMX-specific inhibitor and the cancer cells of the second sample with a HDM2/HDMX dual inhibitor; determining that the cancer cells of the first sample are killed substantially the same or better than the cancer cells of the second sample; and selecting the HDMX-specific inhibitor for treatment of the cancer.

Also featured herein is a stabilized peptide comprising an internally cross-linked polypeptide comprising the amino acid sequence Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆- (SEQ ID NO: 147), wherein:
Xaa₁ is Leu or a conservative amino acid substitution thereof, or Gln or a conservative amino acid substitution thereof, or is missing;
Xaa₂ is Ser or a conservative amino acid substitution thereof, or is missing;
Xaa₃ is Gln or a conservative amino acid substitution thereof, or is missing;
Xaa₄ is Glu or a conservative amino acid substitution thereof, Leu, Ala, or Gln or a conservative amino acid substitution thereof,;
Xaa₅ is Thr or a conservative amino acid substitution thereof, or Ala;
Xaa₆ is Phe or a conservative substitution thereof, Ala, Arg, Glu, or naphthalene;
Xaa₇ is Ser or Glu;
Xaa₈ is Asp or a conservative amino acid substitution thereof, Glu, Ala, or Asn or a conservative amino acid substitution thereof;
Xaa₉ is Leu or a conservative amino acid substitution thereof,, Tyr, or Arg;
Xaa₁₀ is Trp or a conservative substitution thereof, Ala, Arg, or Glu;
Xaa₁₁ is Lys or a conservative amino acid substitution thereof, Ala, Arg or a conservative amino acid substitution thereof, or Glu;
Xaa₁₂ is Leu or a conservative amino acid substitution thereof, Gln, Lys, or Ala;
Xaa₁₃ is a charged amino acid, a hydrophobic amino acid less hydrophobic than alanine, a hydrophilic amino acid, Ala, Arg, Glu, E(OMe), Met(O), homoglutamic acid, 5-fluoronorvaline, Gly, His, Lys, Asn, Gln, Ser, Thr, Val, Trp, Tyr, Pro, or Cys;
Xaa₁₄ is Pro or Thr;
Xaa₁₅ is Glu or a conservative amino acid substitution thereof, Ser, Gln or a conservative amino acid substitution thereof, or Ala; and/or
Xaa₁₆ is Asn or a conservative amino acid substitution thereof, or Ala, or is missing.

In certain embodiments, the side chains of at least two amino acids of the amino acid sequence separated by three or six amino acids are replaced by an internal crosslink, and wherein the internally cross-linked polypeptide preferentially binds HDMX over HDM2. In certain embodiments, in the stabilized peptide, the internally cross-linked polypeptide is alpha helical, neutral to positively charged, cell permeable, and/or not ubiquitinylated. In certain embodiments, Xaa₇ and Xaa₁₄ are stapling amino acids. In certain embodiments, Xaa₇ is (R)-α-(7′-octenyl)alanine and Xaa₁₄ is (S)-α-(4′-pentenyl)alanine, or wherein Xaa₇ is (R)-α-(4′-pentenyl)alanine and Xaa₁₄ is (S)-α-(7′-octenyl)alanine. In certain embodiments, the internally cross linked peptide is 17 to 50 amino acids in length.

In certain embodiments, this disclosure features a structurally-stabilized peptide that preferentially binds to HDMX over HDM2, wherein the structurally -stabilized peptide comprises the amino acid sequence LTFEEYWAQBTSAA (SEQ ID NO: 101), with 3 to 10 amino acid substitutions relative to the amino acid sequence of SEQ ID NO:101, wherein at least two of the 3 to 10 amino acid substitutions are substitutions with stapling amino acids, wherein one of the 3 to 10 amino acid substitutions is a substitution of the amino acid corresponding to L26 of SEQ ID NO:102 with a charged amino acid, alanine, or a hydrophilic amino acid, and optionally wherein the 3 to 10 amino acid substitutions comprises (i) substitution of the amino acid corresponding to F19 of SEQ ID NO:102 with a charged amino acid or alanine, or (ii) substitution of the amino acid corresponding to W23 of SEQ ID NO:102 with a charged amino acid, alanine, or an aromatic amino acid, wherein the structurally-stabilized peptide is stapled or stitched. In certain embodiments, the structurally-stabilized peptide comprises the amino acid sequence of any one of SEQ ID NO: 59, 80, or 86.

In certain embodiments, this disclosure features a structurally-stabilized peptide that preferentially binds to HDMX over HDM2, wherein the structurally-stabilized peptide comprises the amino acid sequence QSQQTFSNLWRLLPQN (SEQ ID NO:100), with 3 to 10 amino acid substitutions relative to the amino acid sequence of SEQ ID NO: 100, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with a charged amino acid, alanine, or a hydrophilic amino acid, and optionally wherein the 3 to 10 amino acid substitutions comprises: (i) substitution of the amino acid corresponding to F19 of SEQ ID NO:102 with a charged amino acid or alanine, or (ii) substitution of the amino acid corresponding to W23 of SEQ ID NO:102 with a charged amino acid, alanine, or an aromatic amino acid, wherein the structurally-stabilized peptide is stapled or stitched. In certain embodiments, the structurally-stabilized peptide comprises the amino acid sequence of any one of SEQ ID NO: 96 or 97.

Also featured herein is a compound comprising a structurally-stabilized peptide comprising a cross-linked amino acid sequence having the formula:

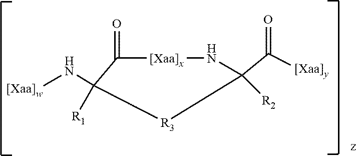

or a pharmaceutically acceptable salt thereof. In certain embodiments, each R1 and R2 is independently H, alkyl, alkenyl, alkynyl, arylalkyl, cycloalkylalkyl, heteroarylalkyl, or heterocyclylalkyl, any of which is substituted or unsubstituted. In certain embodiments, each R3 is independently alkylene, alkenylene, or alkynylene, any of which is substituted or unsubstituted. In certain embodiments, the cross-linked amino acid sequence has the sequence set forth in SEQ ID NO:100 or 101 with at least two amino acid substitutions, where the at least two amino acid substitutions include substitutions of two amino acids in SEQ ID NO:100 or 101 with a stapling amino acid the side chains of which are cross-linked, wherein the structurally-stabilized peptide selectively binds HDMX over HDM2, and optionally wherein the amino acid sequence includes Phe19 and Trp23 of wild type p53. In certain embodiments, in the compound or the pharmaceutically acceptable salt thereof, at least two amino acid substitutions are 3 to 10 amino acid substitutions, optionally 3 to 7, 3 to 6, 3 to 5 or 4 amino acid substitutions. In certain embodiments, [Xaa]_{w} is LTF, [Xaa]ₓ is EYWAQE (SEQ ID NO:194), and [Xaa]_{y} is SAA of the compound or the pharmaceutically acceptable salt thereof. In certain embodiments, [Xaa]_{w} is LTF, [Xaa]ₓ is EYWAQ# (SEQ ID NO:228), and [Xaa]_{y} is SAA of the compound or the pharmaceutically acceptable salt thereof; wherein # is E(OMe). In certain embodiments, [Xaa]_{w} is LTF, [Xaa]ₓ is EYWAQf (SEQ ID NO:218) of the compound or the pharmaceutically acceptable salt thereof, and [Xaa]_{y} is SAA; wherein f is 5-fluoronorvaline. In certain embodiments, [Xaa]_{w} is LTF, [Xaa]ₓ is EYWAQ$, (SEQ ID NO:227) and [Xaa]_{y} is SAA of the compound or the pharmaceutically acceptable salt thereof; wherein $ is methionine-sulfoxide. In certain embodiments, [Xaa]_{w} is QSQQTF (SEQ ID NO:225), [Xaa]ₓ is NLWRLE (SEQ ID NO:226) of the compound or the pharmaceutically acceptable salt thereof, and [Xaa]_{y} is QN. In certain embodiments, [Xaa]_{w} is QSQQTF (SEQ ID NO:225), [Xaa]ₓ is NLWRL# (SEQ ID NO:237), and [Xaa]_{y} is QN of the compound or the pharmaceutically acceptable salt thereof; wherein # is E(OMe). In certain embodiments, the pharmaceutically acceptable salt is acetate, sulfate, or chloride.

Also featured herein is a structurally-stabilized peptide comprising a peptide set forth in any one of SEQ ID NOs.: 59, 80, 86, 96, or 97 with 1 to 8 (1, 2, 3, 4, 5, 6, 7, 8) amino acid substitutions, wherein the substitutions are not at locations of stapling amino acids in the peptide, and optionally wherein the peptide includes Phe19 and Trp23 of wild type p53. In certain embodiments, any of the structurally-stabilized peptides disclosed herein has a length of 14-50 amino acids. In certain embodiments, in the structurally-stabilized peptide disclosed herein, 1 to 7, 1 to 6, 1 to 5, 1 to 4, 1 to 3, 1 to 2, or 1 amino acid substitutions are on the non-interacting face of the helix. In certain embodiments, in the structurally-stabilized peptide disclosed herein, 1 to 5, 1 to 4, 1 to 3, 1 to 2, or 1 of the amino acids of the interacting face are substituted. Also featured herein is a method of treating a HDMX-expressing or dependent cancer in a human subject, the method comprising administering to the human subject a therapeutically effective amount of any of the compounds or the pharmaceutically acceptable salts thereof disclosed herein, or the structurally-stabilized peptides disclosed herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the disclosure will be apparent from the following detailed description and from the claims.

### BRIEF DESCRIPTION OF THE DRAWINGS

FIG. 1 is a drawing summarizing a major discovery of the disclosure: identification of binding determinants that can convert a naturally dual-targeting MDM2/MDMX stapled peptide inhibitor (left) into an MDMX-selective agent (right) for cancer diagnosis and therapy.

FIG. 2 shows an alignment of the human p53 transactivation domain (SEQ ID NO:99) and the unstapled precursors of SAH-p53-4, ATSP-7041, and SAH-p53-8 peptides (i.e., SEQ ID NOs:99, 101, and 100, respectively). The amino acid position (numbered according to SEQ ID NO:102) is shown at the top. The single-underlined amino acids correspond to R8 in the stapled versions of SAH-p53-4, ATSP-7041, and SAH-p53-8. The double-underlined amino acids correspond to S5 in the stapled versions of SAH-p53-4, ATSP-7041, and SAH-p53-8.

FIG. 3A shows the amino acid sequences of a library of stapled p53 peptides based on the SAH-p53-4 sequence (SEQ ID NO:1), including comprehensive alanine, arginine, and glutamate scans for selective HDMX inhibitor discovery.

FIG. 3B shows the amino acid sequences of a library of stapled p53 peptides based on the SAH-p53-4 sequence (SEQ ID NO:1), including double and triple combination mutants for selective HDMX inhibitor discovery.

FIG. 4A shows the amino acid sequences of a library of stapled p53 peptides based on the ATSP-7041 sequence (SEQ ID NO:55), and iterating amino acid position 26 across all natural amino acids and generating single and double non-natural point mutant variants to optimize HDMX-selectivity and cellular penetrance.

FIG. 4B shows the amino acid sequence of an exemplary HDMX-selective stapled p53 peptide (SEQ ID NO:96) based on the SAH-p53-8 sequence (SEQ ID NO:95) and the use of glutamate protection strategies (SEQ ID NOs:97 and 98) to mask negative charge and thereby optimize cellular penetrance. The E(OMe) only temporarily sequesters the negative charge of the glutamate for the purposes of cellular import. Once inside the cell the derivatization comes off (naturally) - for example by hydrolysis of the ester back to the carboxylic acid - yielding the original selective HDMX-inhibitory peptide (SEQ ID NO:96).

FIG. 5A shows the results of competitive binding of the library of biotinylated SAH-p53-4 alanine scanning peptides (see FIG. 3A) to recombinant HDMX (amino acids 1-490 of SEQ ID NO:104) and HDM2 (amino acids 17-125 of SEQ ID NO:103), which revealed exemplary HDMX-selective constructs (e.g., SAH-p53-4 L26A (SEQ ID NO:13), marked by an asterisk), as assessed by competitive streptavidin pull-down of biotinylated peptides incubated with a 1:1 mixture of recombinant HDM2 and HDMX proteins, followed by HDM2 and HDMX western analyses. The peptides tested were biotinylated stapled forms of SEQ ID NOs: 1-15, from left to right (starting at SAH-p53-4), respectively.

FIG. 5B shows the results of competitive binding of the library of biotinylated SAH-p53-4 arginine scanning peptides (see FIG. 3A) to recombinant HDMX (amino acids 1-490 of SEQ ID NO:104) and HDM2 (amino acids 17-125 of SEQ ID NO:103), which revealed exemplary HDMX-selective constructs (e.g., SAH-p53-4 L22R (SEQ ID NO:23), SAH-p53-4 L26R (SEQ ID NO:27), marked by asterisks), as assessed by competitive streptavidin pull-down of biotinylated peptides incubated with a 1:1 mixture of recombinant HDM2 and HDMX proteins, followed by HDM2 and HDMX western analyses. The peptides tested were biotinylated stapled forms of SEQ ID NOs:1 and 16-29, from left to right (starting at SAH-p53-4), respectively.

FIG. 5C shows the results of competitive binding of the library of biotinylated SAH-p53-4 glutamate scanning peptides (see FIG. 3A to recombinant HDMX (amino acids 1-490 of SEQ ID NO:104) and HDM2 (amino acids 17-125 of SEQ ID NO:103), which revealed exemplary HDMX-selective constructs (e.g., SAH-p53-4 L26E (SEQ ID NO:40), marked by an asterisk), as assessed by competitive streptavidin pull-down of biotinylated peptides incubated with a 1:1 mixture of recombinant HDM2 and HDMX proteins, followed by HDM2 and HDMX western analyses. The peptides tested were biotinylated stapled forms of SEQ ID NOs: 1, 30-32, 1, 33-40, 1, and 41, from left to right (starting at SAH-p53-4), respectively.

FIGS. 6A-6F show two quantitative binding analyses of the interactions between the HDMX-selective SAH-p53-4 peptides and humanized zebrafish HDMX protein (amino acids 15-106 L46V/V95L, denoted as zHDMX), using isothermal calorimetry (A-E, top) and fluorescence polarization (A-E, bottom) assays, and a summary table of values (F).

FIGS. 7A-7D show comparative fluorescence polarization binding analyses of the SAH-p53-4 and SAH-p53-4 L26E interactions with zHDMX (A) vs. HDM2 (B). Whereas L26E mutagenesis has little to no effect on zHDMX binding, the same mutation markedly impairs HDM2 interaction. Isothermal calorimetry analyses of the peptide interactions with HDM2 are shown in 7C and 7D.

FIG. 8 shows that the L26E selectivity determinant identified for SAH-p53-4 likewise transforms SAH-p53-8 from a dual-targeting HDM2/HDMX compound into an HDMX-selective agent. The peptides tested were biotinylated stapled forms of SEQ ID NOs: 1, 40, 95, and 96, from left to right (starting at SAH-p53-4), respectively.

FIG. 9 shows that upon replacing the non-natural amino acid cyclobutyl alanine of ATSP-7041 (SEQ ID NO:55), a dual-HDM2/HDMX targeting compound, with each of the natural amino acids or norleucine, the position 26 mutational tolerance of HDM2 is significantly restricted compared to HDMX. For example, HDM2 can only bind to those ATSP-7041 mutants bearing the hydrophobic residues phenylalanine, isoleucine, leucine, or norleucine. In contrast, the HDMX binding pocket can tolerate a much broader spectrum of mutations, including alanine, arginine, and glutamate mutations that engage HDMX with greatest affinity but essentially abrogate HDM2 interaction. The peptides tested were biotinylated stapled forms of SEQ ID NOs: 55-75, from left to right (starting at B26B, SEQ ID NO:55), respectively. b = norleucine.

FIG. 10 presents the crystal structures of SAH-p53-4 (left), SAH-p53-8 (middle), and ATSP-7041 (PDB ID 4NT5) (right) peptides in complex with a recombinant protein construct of HDMX (humanized zebrafish HDMX, denoted as zHDMX), with the insets below highlighting how the hydrophobic residues at position 26 (leucines for SAH-p53-4 and SAH-p53-8; cyclobutyl alanine for ATSP-7041) insert into a hydrophobic cleft of the zHDMX groove.

FIG. 11 presents the crystal structures of SAH-p53-4 L26E (left), SAH-p53-8 L26E (middle), and ATSP-7041 B26E (right) peptides in complex with a recombinant protein construct of HDMX (humanized zebrafish HDMX, denoted as zHDMX), with the insets below highlighting how the zHDMX-binding pocket can accommodate to the installation of a glutamate residue at position 26 through an alternative binding interaction with Tyr96 (numbering based on humanized zebrafish HDMX construct).

FIG. 12 shows that the analogous tyrosine in HDM2 (Y99) is displaced distally (and interacts with residue N29 of the peptide [not shown]) compared to the tyrosine 96 location in humanized zebrafish HDMX (Y99 in HDMX) and that a local histidine in HDM2 (H96) is predicted to clash with the installed glutamate, suggesting why HDM2 cannot accommodate L26E or B26E mutagenesis of the stapled p53 peptides.

FIG. 13 compares the distinct positioning of SAH-p53-8 L26 in a hydrophobic pocket within the zHDMX groove to the alternate interaction of SAH-p53-8 L26E with Y96 of the groove (numbering based on humanized zebrafish HDMX).

FIG. 14 shows that biotinylated SAH-p53-8 (SEQ ID NO:95) can effectively bind to wild-type, Y99F, and Y99A HDMX, whereas the Y99F and Y99A point mutations markedly impair interaction with SAH-p53-8 L26E (SEQ ID NO:96), as monitored by streptavidin pull down and HDMX western analysis. These data again highlight the critical role of Y99 in conferring HDMX binding selectivity to SAH-p53-8 L26E.

FIG. 15 shows that biotinylated SAH-p53-4 (SEQ ID NO:1) can effectively bind to wild-type, Y99F, and Y99A HDMX, whereas the Y99F and Y99A point mutations markedly impair interaction with SAH-p53-4 L26E (SEQ ID NO:40), as monitored by streptavidin pull down and HDMX western analysis. These data again highlight the critical role of Y99 in conferring HDMX binding selectivity to SAH-p53-4 L26E.

FIG. 16A shows that SAH-p53-8 (SEQ ID NO:95) binds to both native HDM2 and HDMX in EOL-1 and OCI-AML2 leukemia cell lysates, whereas L26E mutagenesis (SEQ ID NO:96) abrogates HDM2 binding, yielding selective interaction with HDMX, as monitored by streptavidin pull down of the biotinylated peptides and HDM2 and HDMX Western analyses.

FIG. 16B shows that SAH-p53-8 (SEQ ID NO:95) binds to both native HDM2 and HDMX in the SJSA-X osteosarcoma cell lysates engineered to express HDMX along with endogenous HDM2, whereas L26E mutagenesis (SEQ ID NO:96) impairs HDM2 interaction resulting in predominant HDMX interaction, as assessed by streptavidin pull-down of biotinylated peptides and HDM2 and HDMX western analyses.

FIG. 17A shows how the combination of specificity mutants in SAH-p53-4 can preserve or enhance HDMX binding selectivity, whereas some combinations can unexpectedly restore dual HDM2/HDMX interaction, as evidenced in particular for the K24E/E28A mutant (SEQ ID NO:47) that confers especially robust HDM2 interaction along with HDMX binding, as assessed by competitive streptavidin pull-down of biotinylated peptides incubated with a 1:1 mixture of recombinant HDM2 and HDMX proteins, followed by HDM2 and HDMX western analyses. The peptides tested were biotinylated stapled forms of SEQ ID NOs: 42-44, 48, 45-47, and 49-51, from left to right, respectively.

FIG. 17B shows how select single and double mutants of ATSP-7041 can influence the binding specificity toward HDMX, with select mutants conferring dual HDM2/HDMX binding selectivity (e.g., A24E (SEQ ID NO:77), S28A (SEQ ID NO:79), A24E/S28E (SEQ ID NO:81)), while others preserving or enhancing HDMX binding selectivity, as assessed by competitive streptavidin pull-down of biotinylated peptides incubated with a 1:1 mixture of recombinant HDM2 and HDMX proteins, followed by HDM2 and HDMX western analyses. Top: The peptides tested were biotinylated stapled forms of SEQ ID NOs: 55, 77, 57-59, 79, 81, and 76, from left to right, respectively. Bottom: The peptides tested were biotinylated stapled forms of SEQ ID NO: 55, SEQ ID NO:59, (SEQ ID NO:84)-napthalene-(SEQ ID NO:238), SEQ ID NOs:78, 94, 87, 88, 91, 89, 92, 90, and 93, from left to right, respectively.

FIG. 17C shows how B26E mutagenesis confers HDMX-specificity to ATSP-7041 but decreases the overall level of HDMX interaction, which can be restored while preserving selectivity by replacing B26 with a 5-fluoronorvaline moiety in place of glutamate. L26E mutagenesis of SAH-p53-4 and SAH-p53-8 confers selectivity for HDMX while preserving robust HDMX-binding activity, as assessed by competitive streptavidin pull-down of biotinylated peptides incubated with a 1:1 mixture of recombinant HDM2 and HDMX proteins, followed by HDM2 and HDMX western analyses. The peptides tested were biotinylated stapled forms of SEQ ID NOs: 1, 40, 95, 96, 55, 59, 86, from left to right (starting at SAH-p53-4), respectively.

FIG. 18A shows the cytotoxicity of the selective HDMX-inhibitor compound ATSP-7041 B26E in OCI-AML2 cancer cells, and how esterification of the glutamate residue or replacement with a 5-fluoronorvaline (f) group can enhance killing activity, as shown at 48 and 72 hours of treatment. For both top and bottom panels: The peptides tested were acetylated stapled forms of SEQ ID NOs: 59, 80, and 86, from top to bottom, respectively.

FIG. 18B shows the cytotoxicity of the selective HDMX-inhibitor compound ATSP-7041 B26E in EOL-1 cancer cells, and how esterification of the glutamate residue or replacement with a 5-fluoronorvaline (f) group can enhance killing activity, as shown at 48 and 72 hours of treatment. For both top and bottom panels: The peptides tested were acetylated stapled forms of SEQ ID NOs: 59, 80, and 86, from top to bottom, respectively.

FIG. 18C shows the cytotoxicity of the selective HDMX-inhibitor compound ATSP-B26f (SEQ ID NO: 86), where f is a 5-fluoronorvaline (f), across a spectrum of hematologic and solid malignancies, as shown at 72 hours of treatment.

FIG. 19 shows that at the highest dosing level shown in FIG. 18A and FIG. 18B, the ATSP-7041 B26E (SEQ ID NO:59), B26E(OMe) (SEQ ID NO:80), and B26f (SEQ ID NO:86) mutant compounds cause no non-specific membrane disruptive activity, as monitored by LDH release assay performed at 90 minutes after peptide treatment of OCI-AML2 (FIG. 19A) and EOL-1 (FIG. 19B) cells. The peptides tested were acetylated stapled forms of SEQ ID NOs: 59, 80, and 86, from left to right (starting at ATSP B26E), respectively.

FIG. 20 shows the variety of stapling amino acids that can be used to generate all-hydrocarbon stapled p53 peptides, and the placement of such non-natural amino acids at [i, i+ 3], [i, i+4], or [i, i+7] positions along the length of the peptide sequence followed by crosslinking by ruthenium-catalyzed olefin metathesis.

FIG. 21 illustrates how in addition to single staple insertion and scanning to identify optimal stapled p53 peptides, double and triple stapling can also be accomplished using combinations of [i, i+3], [i, i+4], and [i, i+7] staples.

FIG. 22 illustrates that all-hydrocarbon stitching by use of a Bis-pentenyl glycine moiety can enable two contiguous staples to emerge from a single amino acid position, yielding a diversity of “stitched” p53 peptides.

FIG. 23 illustrates how hydrocarbon-stapling and mutagenesis can be integrated to yield optimal stapled/stitched p53 peptides for therapeutic application.

### DETAILED DESCRIPTION

The present disclosure is based, inter alia, on the surprising discovery that p53 stapled peptides, e.g., p53 stapled peptides initially developed to target both HDM2 and HDMX and whose template sequence naturally binds to both HDM2 and HDMX, may be modified to selectively bind HDMX over HDM2. Accordingly, the present disclosure provides novel methods and compositions (e.g., combinations of compositions) for treating or developing treatments for certain cancers (e.g., HDMX-expressing or -dependent cancers).

The present disclosure is also based, inter alia, on the surprising discovery that p53 stapled peptides, e.g., p53 stapled peptides initially developed to target both HDM2 and HDMX and whose template sequence naturally binds to both HDM2 and HDMX, may be modified to induce an enhanced binding preference for HDM2. Accordingly, the present disclosure also provides novel methods and compositions (e.g., combinations of compositions) for treating or developing treatments for certain cancers (e.g., HDM2-expressing or -dependent cancers).

The present disclosure also provides novel structurally-stabilized (e.g., stapled) p53 peptide compositions that bind both HDM2 and HDMX. Related methods and compositions for treating or developing treatments for certain cancers (e.g., HDM2 and HDMX-expressing or -dependent cancers) are also disclosed.

### P53 Peptides

HDM2 and HDMX are two negative regulators of the tumor suppressor protein p53. Whereas HDM2 binds and destroys p53, HDMX binds and sequesters p53, thereby blocking the anti-cancer activity of p53 and promoting the development, maintenance and chemoresistance of diverse subtypes of adult and pediatric cancers.

An exemplary amino acid sequence of human p53 is shown below (GenBank Accession No. CAA26306) (the transactivation domain is underlined):

MEEPQSDPSVEPPLSQETFSDLWKLLPENNVLSPLPSQAMDDLMLSPDDI
EQWFTEDPGPDEAPRMPEAAPPVAPAPAAPTPAAPAPAPSWPLSSSVPSQ
KTYQGSYGFRLGFLHSGTAKSVTCTYSPALNKMFCQLAKTCPVQLWVDST
PPPGTRVRAMAIYKQSQHMTEWRRCPHHERCSDSDGLAPPQHLIRVEGNL
RVEYLDDRNTFRHSVVVPYEPPEVGSDCTTIHYNYMCNSSCMGGMNRRPI
LTIITLEDSSGNLLGRNSFEVRVCACPGRDRRTEEENLRKKGEPHHELPP
GSTKRALPNNTSSSPQPKKKPLDGEYFTLQIRGRERFEMFRELNEALELK
DAQAGKEPGGSRAHSSHLKSKKGQSTSRHKKLMFKTEGPDSD (SEQ ID NO:102).

The transactivation domain of human p53 consists of amino acids 14 to 29 of SEQ ID NO:102, i.e., the amino acid sequence LSQETFSDLWKLLPEN (SEQ ID NO:99).

An exemplary amino acid sequence of human HDM2 is shown below (GenBank Accession No. NP_002383.2):

MCNTNMSVPTDGAVTTSQIPASEQETLVRPKPLLLKLLKSVGAQKDTYTM
KEVLFYLGQYIMTKRLYDEKQQHIVYCSNDLLGDLFGVPSFSVKEHRKIY
TMIYRNLVVVNQQESSDSGTSVSENRCHLEGGSDQKDLVQELQEEKPSSS
HLVSRPSTSSRRRAISETEENSDELSGERQRKRHKSDSISLSFDESLALC
VIREICCERSSSSESTGTPSNPDLDAGVSEHSGDWLDQDSVSDQFSVEFE
VESLDSEDYSLSEEGQELSDEDDEVYQVTVYQAGESDTDSFEEDPEISLA
DYWKCTSCNEMNPPLPSHCNRCWALRENWLPEDKGKDKGEISEKAKLENS
TQAEEGFDVPDCKKTIVNDSRESCVEENDDKITQASQSQESEDYSQPSTS
SSIIYSSQEDVKEFEREETQDKEESVESSLPLNAIEPCVICQGRPKNGCI
VHGKTGHLMACFTCAKKLKKRNKPCPVCRQPIQMIVLTYFP (SEQ ID NO:103).

An exemplary amino acid sequence of human HDMX is shown below (GenBank Accession No. NP_002384.2):

MTSFSTSAQCSTSDSACRISPGQINQVRPKLPLLKILHAAGAQGEMFTVK
EVMHYLGQYIMVKQLYDQQEQHMVYCGGDLLGELLGRQSFSVKDPSPLYD
MLRKNLVTLATATTDAAQTLALAQDHSMDIPSQDQLKQSAEESSTSRKRT
TEDDIPTLPTSEHKCIHSREDEDLIENLAQDETSRLDLGFEEWDVAGLPW
WFLGNLRSNYTPRSNGSTDLQTNQDVGTAIVSDTTDDLWFLNESVSEQLG
VGIKVEAADTEQTSEEVGKVSDKKVIEVGKNDDLEDSKSLSDDTDVEVTS
EDEWQCTECKKFNSPSKRYCFRCWALRKDWYSDCSKLTHSLSTSDITAIP
EKENEGNDVPDCRRTISAPVVRPKDAYIKKENSKLFDPCNSVEFLDLAHS
SESQETISSMGEQLDNLSEQRTDTENMEDCQNLLKPCSLCEKRPRDGNII
HGRTGHLVTCFHCARRLKKAGASCPICKKEIQLVIKVFIA (SEQ ID NO:104).

ATSP-7041, SAH-p53-4, and SAH-p53-8 are dual HDMX- and HDM2-selective stapled peptides (i.e., structurally-stabilized peptides) based on the human p53 transactivation domain (SEQ ID NO:99). The amino acid sequence of ATSP-7041 is LTF8EYWAQBXSAA (SEQ ID NO:55), wherein each of 8 and X is independently a stapling amino acid and B is cyclobutylalanine. The unstapled precursor amino acid sequence of ATSP-7041 is LTFEEYWAQBTSAA (SEQ ID NO:101). The amino acid sequence of SAH-p53-4 is LSQETF8DLWKLLXEN (SEQ ID NO:1), wherein each of 8 and X is independently a stapling amino acid. The unstapled precursor amino acid sequence of SAH-p53-4 is LSQETFSDLWKLLPEN (SEQ ID NO:99). The amino acid sequence of SAH-p53-8 is QSQQTF8NLWRLLXQN (SEQ ID NO:95), wherein each of 8 and X is independently a stapling amino acid. The unstapled precursor amino acid sequence of SAH-p53-8 is QSQQTFSNLWRLLPQN (SEQ ID NO:100).

Each of SAH-p53-4, ATSP-7041, and SAH-p53-8 can be N-terminal acetylated and/or C-terminal amidated. N-terminal acetylation and C-terminal amidation create modified proteins that mimic the native protein by reducing the overall charge of a peptide, thus increasing the metabolic stability of peptides and the ability to resist enzymatic degradation. N-terminal acetylation is a post-translational modification in which an acetyl group is appended to the N-terminal amino group of a peptide, altering the charge, hydrophobicity, and size of the N-terminus. N-terminal acetylation is catalyzed by N^{α}-acetyltransferases, which accept acetyl-CoA as the donor for the transfer of the activated acetyl moiety to the N^{α}-terminus of the protein (Linster et al., J Exp Bot. 2018 Aug 31;69(19):4555-4568). In some embodiments, an acetylation reaction includes deprotection of an Fmoc group, followed by reaction with an esterification agent (e.g., neat acetic anhydride) and an organic compound (e.g., N,N-Diisopropylethylamine (DIPEA)). C-terminal amidation is a post-translational modification to include an amide group at the C-terminus. In some embodiments, the modified amino acid is followed by a glycine, which provides the amide group. During C-terminal amidation, the glycine is oxidized to form alpha-hydroxy-glycine. The oxidized glycine cleaves into the C-terminally amidated peptide and an N-glyoxylated peptide. In some instances, for peptide synthesis the synthesis is started with RINK-AMIDE resin, which renders the most C-terminal residue “amidated”.

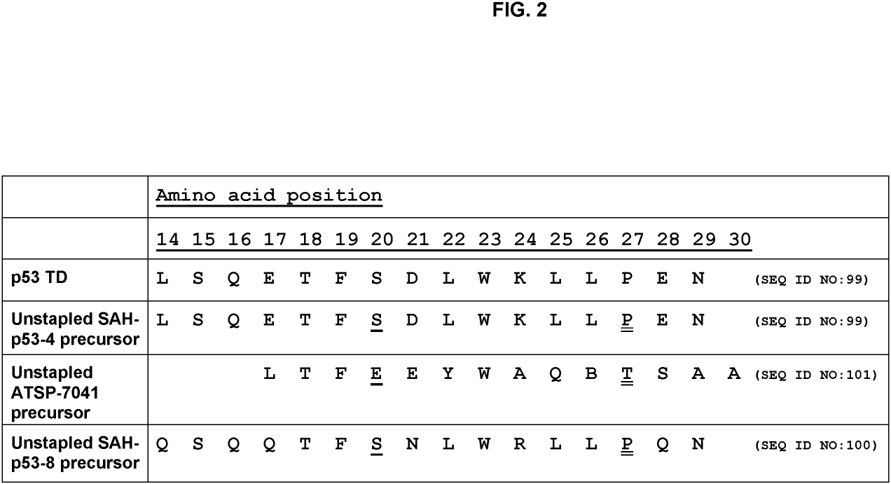

FIG. 2 shows an alignment of the human p53 transactivation domain (SEQ ID NO:99) and the template amino acid sequences upon which the stapled p53-based peptides SAH-p53-4, ATSP-7041, and SAH-p53-8 (i.e., SEQ ID NOs:99, 101, and 100, respectively) are based. Each of these peptides retains the triad of amino acids that are considered to be essential for binding to HDM2 and HDMX: amino acids Phe19 (F19), Trp23 (W23), and Leu26 (L26) (numbered according to the amino acid sequence set forth in SEQ ID NO:102). Phe19 (F19), Trp23 (W23), and Leu26 (L26) (numbered according to the amino acid sequence set forth in SEQ ID NO:102) correspond to positions Phe6 (F6), Trp10 (W10), and Leu13 (L13) of SAH-p53-4 and SAH-p53-8 (i.e., of SEQ ID NOs: 1 and 95, respectively) and positions Phe3 (F3), Trp7 (W7), and Cyclobutylalanine10 (B10) of ATSP-7041 (i.e., of SEQ ID NO:55).

Provided herein are p53 peptides comprising a modified amino acid sequence of the p53 transactivation domain (SEQ ID NO:99), ATSP-7041 (SEQ ID NO:55) or its unstapled precursor (SEQ ID NO:101), SAH-p53-4 (SEQ ID NO: 1) or its unstapled precursor (SEQ ID NO:99), or SAH-p53-8 (SEQ ID NO:95) or its unstapled precursor (SEQ ID NO: 100).

### HDMX-Selective Peptides

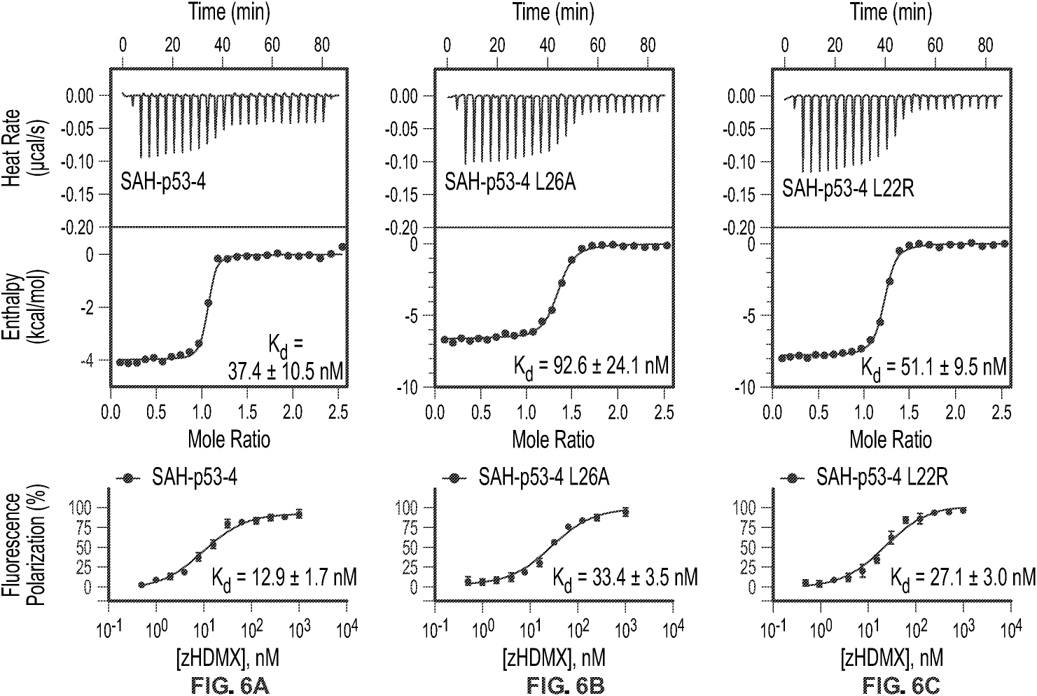

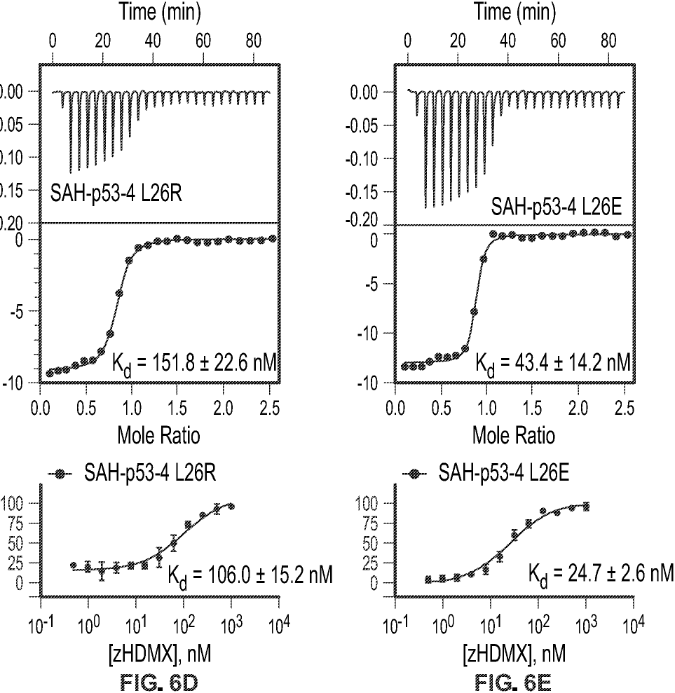

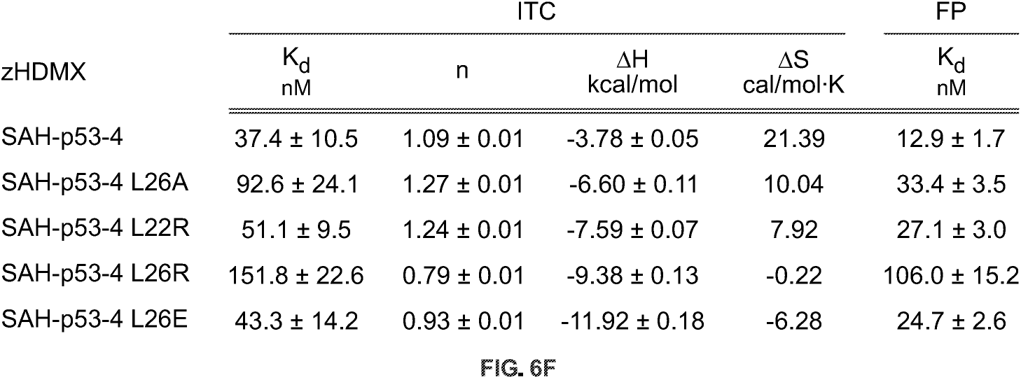

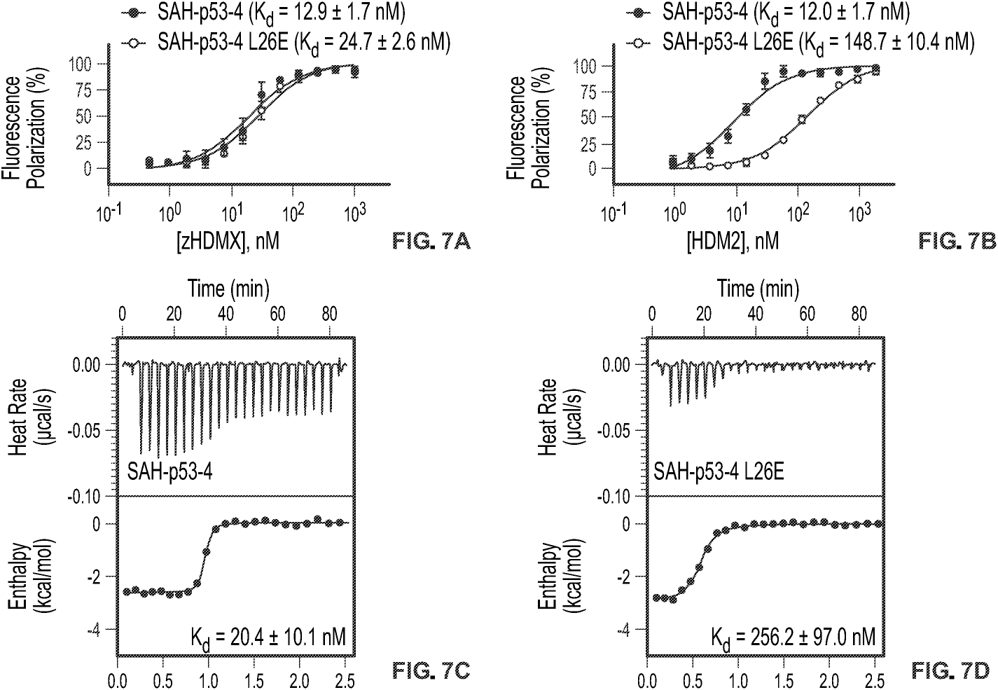

In some aspects, the p53 peptides described herein are based, in part, on the discovery that modification of the p53 triad (Phe19, Trp23, and Leu26 of SEQ ID NO:102) converts p53 peptides into HDMX-selective peptides. In particular, the Examples section below demonstrates that p53 peptides having a substitution of the amino acid corresponding to L26 of SEQ ID NO:102 with an amino acid other than phenylalanine, isoluecine, norleucine, or leucine (e.g., a charged amino acid (e.g., arginine or glutamic acid), alanine, valine, tryptophan, a hydrophobic amino acid less hydrophobic than leucine, or a hydrophilic amino acid) yields a p53 peptide that is selective for HDMX relative to HDM2. In addition, a substitution of the amino acid corresponding to F19 of SEQ ID NO:102 with a charged amino acid or alanine, and/or a substitution of the amino acid corresponding to W23 of SEQ ID NO:102 with a charged amino acid or alanine, can yield a p53 peptide that is selective for HDMX relative to HDM2. Additional amino acid substitutions in p53 conferring HDMX-selectivity (over HDM2) are described in the Examples section below (e.g., L22R, L26A, L26R, and L26E provide strong HDMX-binding selectivity; other p53 transactivation domain peptides that also impart HDMX selectivity over HDM2 include E17A, F19A, D21A, W23A, F19R, W22R, N29R, Q16E, F19E, W23E, K24E; and in the context of ATSP-7041, B26D, B26A, B26R, B26E, B26G, B26H, B26K, B26N, B26Q, B26S, B26T, B26V, B26W, B26Y, B26P, or B26C). One or more (1, 2, 3, 4, 5, 6, 7, 8, 9) of these substitutions may be made in a p53 transactivation domain peptide to enable it to preferentially bind HDMX over HDM2. Thus, provided herein are peptides based on the human p53-transactivation domain (SEQ ID NO:99) that are selective for HDMX over HDM2. In some aspects, a peptide that is selective for HDMX over HDM2 binds to HDMX with at least 1.5-fold, at least 2-fold, at least 2.5-fold, at least 3-fold, at least 4-fold, at least 5-fold, at least 6-fold, at least 7-fold, at least 8-fold, at least 9-fold, at least 10-fold, at least 15-fold, or at least 20-fold greater affinity than to HDM2. Binding affinity may be determined by, e.g., isothermal calorimetry or fluorescence polarization analyses. For example, SAH-p53-4 L26E (SEQ ID:40) displays 12-fold higher binding affinity for HDMX as compared to HDM2, as directly measured by isothermal calorimetry or fluorescence polarization analyses (see FIGS. 6A, 6E, 6F and 7A-7D).

In certain aspects, the HDMX-selective p53 peptide is based on, or derived from, the p53 transactivation domain and includes peptides having the amino acid sequence of positions 14-29 of SEQ ID NO:102 and variants thereof and includes stapled peptides having 3-12 (e.g., 3, 4, 5, 6, 7, 8, 9, 10, 11, or 12) amino acid substitutions in and/or 0-5 (e.g., 0, 1, 2, 3, 4, 5) amino acid additions and/or deletions at the N- and/or C-terminus of the amino acid sequence of SEQ ID NO:99, SEQ ID NO:100, or SEQ ID NO:101, wherein two of the 3-12 amino acid substitutions are with stapling amino acids (e.g., α,α-disubstituted non-natural amino acids with olefinic side chains that can be cross-linked (optionally by ring closing metathesis reaction)) and one of the 3-12 amino acid substitutions is at the amino acid corresponding to Leu26 of SEQ ID NO:102 with any amino acid other than phenylalanine, isoleucine, norleucine, and leucine (e.g., a charged amino acid (e.g., Glu, Gln, Asp, Lys, Arg), alanine, valine, tryptophan, a hydrophobic amino acid less hydrophobic than leucine, or a hydrophilic amino acid, e.g., with alanine, arginine, glutamic acid, valine, tryptophan, serine, histidine, tyrosine, asparagine, glutamine), wherein the peptide preferentially binds HDMX (e.g., has an at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity to HDMX than to HDM2 as determined by, e.g., isothermal calorimetry or fluorescence polarization analyses).

In certain aspects, the HDMX-selective p53 peptide comprises a stabilized p53 peptide that binds HDMX, wherein the stabilized p53 peptide has 3-11 (e.g., 3, 4, 5, 6, 7, 8, 9, 10, 11) amino acid substitutions relative to the sequence of SEQ ID NO:101, 3-13 (e.g., 3, 4, 5, 6, 7, 8, 9, 10, 11, 12, 13) amino acid substitutions relative to the sequence of SEQ ID NO:100, or 3-13 (e.g., 3, 4, 5, 6, 7, 8, 9, 10, 11, 12, 13) amino acid substitutions relative to the sequence of SEQ ID NO:99, wherein at least two of the amino acid substitutions are substitutions of natural amino acids separated by 2, 3, or 6 amino acids with α,α-disubstituted non-natural amino acids with olefinic side chains that can be cross-linked (optionally by ring closing metathesis reaction) and one of the amino acid substitutions is with any amino acid other than phenylalanine, isoleucine, norleucine, and leucine (e.g., a charged amino acid (e.g., Glu, Gln, Asp, Lys, Arg), alanine, valine, tryptophan, a hydrophobic amino acid less hydrophobic than leucine, or a hydrophilic amino acid) at position 26 (numbered according to SEQ ID NO:102), wherein the peptide has 0-5 additions and/or deletions at the N-terminus and/or C-terminus relative to the sequence of SEQ ID NO:101, SEQ ID NO:100, or SEQ ID NO:99, and wherein the peptide preferentially binds HDMX over HDM2 (e.g., has an at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity to HDMX than to HDM2 as determined by, e.g., isothermal calorimetry or fluorescence polarization analyses). In some instances, the peptide comprises an arginine at the amino acid corresponding to position 22 of SEQ ID NO:102. In some instances, the peptide comprises a Phe at the amino acid corresponding to position 19 of SEQ ID NO:102. In some instances, the peptide comprises a tryptophan at the amino acid corresponding to position 23 of SEQ ID NO:102. In some instances, one or more of the negatively charged residues in a peptide described herein are esterified, optionally by adding OMe to one or more of the negatively charged residues. In some instances, the peptide comprises the amino acid sequence of any one of SEQ ID NO:13, 23, 27, 40, 42, 45, 58, 51, 56-59, 61, 62, 64, 67-75, 78, 80, 82-94, and 96-98.

In certain aspects, the HDMX-selective p53 peptide comprises the amino acid sequence of the p53 transactivation domain (SEQ ID NO:99), ATSP-7041 (SEQ ID NO:55) or its unstapled precursor (SEQ ID NO:101), SAH-p53-4 (SEQ ID NO:1) or its unstapled precursor (SEQ ID NO:99), or SAH-p53-8 (SEQ ID NO:95) or its unstapled precursor (SEQ ID NO:100), with 1 to 10 (e.g., 1, 2, 3, 4, 5, 6, 7, 8, 9, or 10) amino acid substitutions, wherein at least one of the 1 to 10 amino acid substitutions is a substitution of the amino acid corresponding to Leu26 of the amino acid sequence of SEQ ID NO:102 with any amino acid other than phenylalanine, isoleucine, norleucine, and leucine (e.g., a charged amino acid (e.g., Glu, Gln, Asp, Lys, Arg), alanine, valine, tryptophan, a hydrophobic amino acid less hydrophobic than leucine, or a hydrophilic amino acid). In some instances, the HDMX-selective p53 peptide comprises a glutamine (Gln, Q) at the amino acid position corresponding to Gln16 of SEQ ID NO:102. In some instances, the HDMX-selective p53 peptide comprises a phenylalanine (Phe, F) at the amino acid position corresponding to Phe19 of SEQ ID NO:102. In some instances, the HDMX-selective p53 peptide comprises an aspartic acid (Asp, D) at the amino acid position corresponding to Asp21 of SEQ ID NO:102. In some instances, the HDMX-selective p53 peptide comprises a tryptophan (Trp, W) at the amino acid position corresponding to Trp23 of SEQ ID NO:102. In some instances, one of the 1 to 10 amino acid substitutions is a conservative amino acid substitution of the amino acid corresponding to Glu16 of SEQ ID NO:102. In some instances, one of the 1 to 10 amino acid substitutions is a conservative amino acid substitution of the amino acid corresponding to Phe19 of SEQ ID NO:102. In some instances, one of the 1 to 10 amino acid substitutions is a conservative amino acid substitution of the amino acid corresponding to Asp21 of SEQ ID NO:102. In some instances, one of the 1 to 10 amino acid substitutions is a conservative amino acid substitution of the amino acid corresponding to Trp23 of SEQ ID NO:102. In some instances, the 1 to 10 amino acid substitutions does not comprise an amino acid substitution that confers HDM2-selectively (see the Examples section below). In some instances, the 1 to 10 amino acid substitutions does not comprise an amino acid substitution that confers dual HDMX-and HDM2-selectivity (see the Examples section below). In some instances, the 1 to 10 amino acid substitutions does not comprise substitution with a proline. In some instances, the 1 to 10 amino acid substitutions does not comprise a substitution of the stapling amino acids in ATSP-7041, SAH-p53-4, or SAH-p53-8 (unless it is with another stapling amino acid that permits the peptide to be stapled).

In certain aspects, the HDMX-selective p53 peptide comprises the amino acid sequence of the p53 transactivation domain (SEQ ID NO:99), ATSP-7041 (SEQ ID NO:55) or its unstapled precursor (SEQ ID NO:101), SAH-p53-4 (SEQ ID NO:1) or its unstapled precursor (SEQ ID NO:99), or SAH-p53-8 (SEQ ID NO:95) or its unstapled precursor (SEQ ID NO:100), with 1 to 10 (e.g., 1, 2, 3, 4, 5, 6, 7, 8, 9, or 10) amino acid substitutions, wherein at least one of the 1 to 10 amino acid substitutions is a substitution of the amino acid corresponding to Leu26 of the amino acid sequence of SEQ ID NO:102 with a charged amino acid, alanine, a hydrophobic amino acid less hydrophobic than leucine, or a hydrophilic amino acid. In some instances, the HDMX-selective p53 peptide comprises a glutamine (Gln, Q) at the amino acid position corresponding to Gln16 of SEQ ID NO:102. In some instances, the HDMX-selective p53 peptide comprises a phenylalanine (Phe, F) at the amino acid position corresponding to Phe19 of SEQ ID NO:102. In some instances, the HDMX-selective p53 peptide comprises an aspartic acid (Asp, D) at the amino acid position corresponding to Asp21 of SEQ ID NO:102. In some instances, the HDMX-selective p53 peptide comprises a tryptophan (Trp, W) at the amino acid position corresponding to Trp23 of SEQ ID NO:102. In some instances, one of the 1 to 10 amino acid substitutions is a conservative amino acid substitution of the amino acid corresponding to Glu16 of SEQ ID NO:102. In some instances, one of the 1 to 10 amino acid substitutions is a conservative amino acid substitution of the amino acid corresponding to Phe19 of SEQ ID NO:102. In some instances, one of the 1 to 10 amino acid substitutions is a conservative amino acid substitution of the amino acid corresponding to Asp21 of SEQ ID NO:102. In some instances, one of the 1 to 10 amino acid substitutions is a conservative amino acid substitution of the amino acid corresponding to Trp23 of SEQ ID NO:102. In some instances, the 1 to 10 amino acid substitutions does not comprise an amino acid substitution that confers HDM2-selectively (see the Examples section below). In some instances, the 1 to 10 amino acid substitutions does not comprise an amino acid substitution that confers dual HDMX- and HDM2-selectivity (see the Examples section below). In some instances, the 1 to 10 amino acid substitutions does not comprise substitution with a proline. In some instances, the 1 to 10 amino acid substitutions does not comprise a substitution of the stapling amino acids in ATSP-7041, SAH-p53-4, or SAH-p53-8 (unless it is with another stapling amino acid that permits the peptide to be stapled).

In certain aspects, the HDMX-selective p53 peptide comprises the amino acid sequence of the p53 transactivation domain (SEQ ID NO:99), ATSP-7041 (SEQ ID NO:55) or its unstapled precursor (SEQ ID NO:101), SAH-p53-4 (SEQ ID NO:1) or its unstapled precursor (SEQ ID NO:99), or SAH-p53-8 (SEQ ID NO:95) or its unstapled precursor (SEQ ID NO:100), with 1 to 10 (e.g., 1, 2, 3, 4, 5, 6, 7, 8, 9, or 10) amino acid substitutions, wherein at least one of the 1 to 10 amino acid substitutions is a substitution of the amino acid corresponding to Phe19 of the amino acid sequence of SEQ ID NO:102 with a charged amino acid or alanine. In some instances, the HDMX-selective p53 peptide comprises a leucine (Leu, L) at the amino acid position corresponding to Leu26 of SEQ ID NO:102. In some instances, the HDMX-selective p53 peptide comprises a tryptophan (Trp, W) at the amino acid position corresponding to Trp23 of SEQ ID NO:102. In some instances, one of the 1 to 10 amino acid substitutions is a conservative amino acid substitution of the amino acid corresponding to Leu26 of SEQ ID NO:102. In some instances, one of the 1 to 10 amino acid substitutions is a conservative amino acid substitution of the amino acid corresponding to Trp23 of SEQ ID NO:102. In some instances, the 1 to 10 amino acid substitutions does not comprise an amino acid substitution that confers HDM2-selectively (see the Examples section below). In some instances, the 1 to 10 amino acid substitutions does not comprise an amino acid substitution that confers dual HDMX- and HDM2-selectivity (see the Examples section below). In some instances, the 1 to 10 amino acid substitutions does not comprise substitution with a proline. In some instances, the 1 to 10 amino acid substitutions does not comprise a substitution of the stapling amino acids in ATSP-7041, SAH-p53-4, or SAH-p53-8 (unless it is with another stapling amino acid that permits the peptide to be stapled).

In certain aspects, the HDMX-selective p53 peptide comprises the amino acid sequence of the p53 transactivation domain (SEQ ID NO:99), ATSP-7041 (SEQ ID NO:55) or its unstapled precursor (SEQ ID NO:101), SAH-p53-4 (SEQ ID NO:1) or its unstapled precursor (SEQ ID NO:99), or SAH-p53-8 (SEQ ID NO:95) or its unstapled precursor (SEQ ID NO:100), with 1 to 10 (e.g., 1, 2, 3, 4, 5, 6, 7, 8, 9, or 10) amino acid substitutions, wherein at least one of the 1 to 10 amino acid substitutions is a substitution of the amino acid corresponding to Trp23 of the amino acid sequence of SEQ ID NO:102 with a charged amino acid, alanine, or an aromatic amino acid. In some instances, the HDMX-selective p53 peptide comprises a phenylalanine (Phe, F) at the amino acid position corresponding to Phe19 of SEQ ID NO:102. In some instances, the HDMX-selective p53 peptide comprises a leucine (Leu, L) at the amino acid position corresponding to Leu26 of SEQ ID NO:102. In some instances, one of the 1 to 10 amino acid substitutions is a conservative amino acid substitution of the amino acid corresponding to Phe19 of SEQ ID NO:102. In some instances, one of the 1 to 10 amino acid substitutions is a conservative amino acid substitution of the amino acid corresponding to Leu26 of SEQ ID NO:102. In some instances, the 1 to 10 amino acid substitutions does not comprise an amino acid substitution that confers HDM2-selectively (see the Examples section below). In some instances, the 1 to 10 amino acid substitutions does not comprise an amino acid substitution that confers dual HDMX-and HDM2-selectivity (see the Examples section below). In some instances, the 1 to 10 amino acid substitutions does not comprise substitution with a proline. In some instances, the 1 to 10 amino acid substitutions does not comprise a substitution of the stapling amino acids in ATSP-7041, SAH-p53-4, or SAH-p53-8 (unless it is with another stapling amino acid that permits the peptide to be stapled).

In certain aspects, the HDMX-selective p53 peptide comprises the amino acid sequence of the p53 transactivation domain (SEQ ID NO:99), ATSP-7041 (SEQ ID NO:55) or its unstapled precursor (SEQ ID NO:101), SAH-p53-4 (SEQ ID NO:1) or its unstapled precursor (SEQ ID NO:99), or SAH-p53-8 (SEQ ID NO:95) or its unstapled precursor (SEQ ID NO:100), with 1 to 10 (e.g., 1, 2, 3, 4, 5, 6, 7, 8, 9, or 10) amino acid substitutions, wherein at least one of the amino acid substitutions is at the amino acid position corresponding to E17, F19, L22, W23, K24, L26, or N29, or combinations thereof, of the amino acid sequence of SEQ ID NO:102. In some aspects, the substitution at the amino acid position corresponding to E17 of the amino acid sequence of SEQ ID NO:102 is with a negatively charged amino acid. In some instances, the substitution at the amino acid position corresponding to E17 of the amino acid sequence of SEQ ID NO:102 is with O-methylated glutamate (E(OMe)). In some aspects, the substitution at the amino acid position corresponding to F19 of the amino acid sequence of SEQ ID NO:102 is with a charged amino acid or alanine. In some instances, the substitution at the amino acid position corresponding to F19 of the amino acid sequence of SEQ ID NO:102 is with alanine (A), arginine (R), or glutamate (E). In some aspects, the substitution at the amino acid position corresponding to L22 of the amino acid sequence of SEQ ID NO:102 is with a positively charged amino acid. In some instances, the substitution at the amino acid position corresponding to L22 of the amino acid sequence of SEQ ID NO:102 is with arginine (R). In some aspects, the substitution at the amino acid position corresponding to W23 of the amino acid sequence of SEQ ID NO:102 is with a charged amino acid, alanine, or an aromatic amino acid. In some instances, the substitution at the amino acid position corresponding to W23 of the amino acid sequence of SEQ ID NO:102 is with naphthalene, alanine (A), arginine (R), or glutamate (E). In some aspects, the substitution at the amino acid position corresponding to K24 of the amino acid sequence of SEQ ID NO:102 is with a charged amino acid or an aromatic amino acid. In some instances, the substitution at the amino acid position corresponding to K24 of the amino acid sequence of SEQ ID NO:102 is with arginine (R), leucine (L), phenylalanine (F), or glutamate (E). In some aspects, the substitution of the amino acid corresponding to Leu26 of the amino acid sequence of SEQ ID NO:102 is with any amino acid other than phenylalanine, isoleucine, norleucine, and leucine. In some aspects, the substitution at the amino acid position corresponding to L26 of the amino acid sequence of SEQ ID NO:102 is with a charged amino acid, alanine, a hydrophobic amino acid less hydrophobic than leucine, or a hydrophilic amino acid. In some instances, the substitution at the amino acid position corresponding to L26 of the amino acid sequence of SEQ ID NO:102 is with valine or tryptophan. In some instances, the substitution at the amino acid position corresponding to L26 of the amino acid sequence of SEQ ID NO:102 is with a hydrophobic amino acid less hydrophobic than leucine. In some instances, the substitution at the amino acid position corresponding to L26 of the amino acid sequence of SEQ ID NO:102 is with alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C). In some aspects, the substitution at the amino acid position corresponding to N29 of the amino acid sequence of SEQ ID NO:102 is with a positively charged amino acid or an aromatic amino acid. In some instances, the substitution at the amino acid position corresponding to N29 of the amino acid sequence of SEQ ID NO:102 is with leucine (L), phenylalanine (F), or arginine (R). In some instances, the 1 to 10 amino acid substitutions consists of a substitution at the amino acid position corresponding to L26 of the amino acid sequence of SEQ ID NO:102 with any amino acid other than than phenylalanine, isoleucine, norleucine, and leucine. In some instances, the 1 to 10 amino acid substitutions consists of a substitution at the amino acid position corresponding to L26 of the amino acid sequence of SEQ ID NO:102 with a charged amino acid, alanine, a hydrophobic amino acid less hydrophobic than leucine, or a hydrophilic amino acid. In some instances, the 1 to 10 amino acid substitutions consists of a substitution at the amino acid position corresponding to L26 of the amino acid sequence of SEQ ID NO:102 with valine or tryptophan. In some instances, the 1 to 10 amino acid substitutions consists of a substitution at the amino acid position corresponding to L26 of the amino acid sequence of SEQ ID NO:102 with a hydrophobic amino acid less hydrophobic than leucine. In some instances, the 1 to 10 amino acid substitutions consists of a substitution at the amino acid position corresponding to L26 of the amino acid sequence of SEQ ID NO:102 is with alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C). In some instances, the 1 to 10 amino acid substitutions does not comprise an amino acid substitution that confers HDM2-selectively (see the Examples section below). In some instances, the 1 to 10 amino acid substitutions does not comprise an amino acid substitution that confers dual HDMX- and HDM2-selectivity (see the Examples section below). In some instances, the 1 to 10 amino acid substitutions does not comprise substitution with a proline. In some instances, the 1 to 10 amino acid substitutions does not comprise a substitution of the stapling amino acids in ATSP-7041, SAH-p53-4, or SAH-p53-8 (unless it is with another stapling amino acid that permits the peptide to be stapled).

Non-limiting examples of hydrophobic amino acids include glycine (Gly), alanine (Ala), valine (Val), leucine (Leu), isoleucine (Ile), proline (Pro), phenylalanine (Phe), methionine (Met), and tryptophan (Trp). Non-limiting examples of hydrophilic amino acids include threonine (Thr), serine (Ser), lysine (Lys), glutamic acid (Gln), asparagine (Asn), histidine (His), glutamic acid (Glu), aspartic acid (Asp) and arginine (Arg). Non-limiting examples of aromatic amino acids include phenylalanine, tyrosine, histidine, and tryptophan, and the non-natural aromatic amino acid naphthalene. The hydrophobicity of an amino acid may be classified according to, e.g., the Eisenberg scale (Eisenberg et al., (1984), J Mol Biol, 179(1): 125-142), which is incorporated by refereince herein in its entirety. In some instances, the charged amino acids are Glu, Gln, Asp, Lys, and Arg. In some instances, the polar/uncharged amino acids are Cys, Tyr, Thr, Ser, Asn, and Gln.

A “conservative amino acid substitution” means that the substitution replaces one amino acid with another amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), aromatic side chains (e.g., tyrosine, phenylalanine, tryptophan, histidine), and acidic side chains and their amides (e.g., aspartic acid, glutamic acid, asparagine, glutamine).

In certain aspects, the HDMX-selective p53 peptide comprises or consists of an amino acid sequence set forth in Table 1.

TABLE 1

Exemplary HDMX-selective p53 peptides.

| Description | Sequence | SEQ ID NO |
|---|---|---|
| SAH-p53-4 Q16E | LSEETFSDLWKLLPEN | 105 |
| SAH-p53-4 E17A | LSQATFSDLWKLLPEN | 106 |
| SAH-p53-4 F19(A/R/E) | LSQETX₁SDLWKLLPEN, wherein X₁ is A, R, or E | 107 |
| SAH-p53-4 D21A | LSQETFSALWKLLPEN | 108 |
| SAH-p53-4 L22R | LSQETFSDRWKLLPEN | 109 |
| SAH-p53-4 W23(A/R/E) | LSQETFSDLX₁KLLPEN, wherein X₁ is A, R, or E | 110 |
| SAH-p53-4 K24E | LSQETFSDLWELLPEN | 111 |
| SAH-p53-4 K24E/L26(A/R/E) | LSQETFSDLWELX₁PEN, wherein X₁ is A, R, or E | 112 |
| SAH-p53-4 K24E/L26(A/R/E)/E28A | LSQETFSDLWELX₁PAN, wherein X₁ is A, R, or E | 113 |
| SAH-p53-4 L26(A/R/E) | LSQETFSDLWKLX₁PEN, wherein X₁ is A, R, or E | 114 |
| SAH-p53-4 L26(A/R/E)/E28A | LSQETFSDLWKLX₁PAN, wherein X₁ is A, R, or E | 115 |
| SAH-p53-4 N29R | LSQETF SDLWKLLPER | 116 |
| ATSP-7041 B26(D/A/R/E/G/H/K/N/Q/S/T/VW/Y/P/C/h/#/f) | LTFEEYWAQX₁TSAA, wherein X₁ is D, A, R, E, G, H, K, N, Q, S, T, V, W, Y, P, C, homoglutamic acid, E(OMe), or 5-fluoronorvaline | 117 |
| ATSP-7041 W23Z/B26E | LTFEEYX₁AQETAAA, wherein X₁ is naphthalene | 118 and 239 |
| ATSP-7041 A24(R/L/F)/B26E | LTFEEYWX₁QETAAA, wherein X₁ is R, L, or F | 119 |
| ATSP-7041 B26E/A29(L/F) | LTFEEYWAQETSX₁A, wherein X₁ is L or F | 120 |
| ATSP-7041 E21(L/F)/B26E | LTFEX₁YWAQETSAA, wherein X₁ is L or F | 121 |
| ATSP-7041 Y22R/B26E | LTFEERWAQETSAA | 122 |
| SAH-p53-8 L26(E/#) | QSQQTFSNLWRLX₁PQN, wherein X₁ is E or E(OMe) | 123 |
| SAH-p53-8 Q17# / L26# | QSQX₁TFS8NLWRLX₁PQN, wherein X₁ is E(OMe) | 124 |

In certain instances, the HDMX-selective p53 peptides described herein may also contain one or more (e.g., 1, 2, 3, 4, 5, 6, 7, 8, 9, 10) amino acid substitutions (relative to an amino acid sequence set forth in any one of SEQ ID NOs: 105-124), e.g., one or more (e.g., 1, 2, 3, 4, 5, 6, 7, 8, 9, 10) conservative and/or non-conservative amino acid substitutions. In certain instances, these substitutions are at the amino acid positions corresponding to positions Q16, E17, F19, D21, L22, W23, K24, L26, E28, or N29 of SEQ ID NO:102. In certain instances, these substitution(s) are of amino acids on the interacting face of the p53 peptide. In certain instances, these substitution(s) are of amino acids on the non-interacting face of the p53 peptide. In certain instances, these substitutions are of both amino acids on the interacting face of the p53 peptide and on the non-interacting face of the p53 peptide. Just about every one of the amino acids on the non-interacting face of the peptide can be varied. In certain instances, the substituted amino acid(s) are selected from the group consisting of L-Ala, D-Ala, Aib, Ser, a substituted alanine, or a substituted glycine derivative.

The “interacting face” of the p53 peptides described herein includes those amino acid residues of the p53 transactivation domain (SEQ ID NO:99) that interact (e.g., interact specifically or bind specifically) with HDM2 and/or HDMX. Amino acid residues contained within the interacting face of p53, including amino acid residues contained within the interacting face of the p53 transactivation domain, are known in the art (see, e.g., Kussie et al., Science, 274(5289):948-953 (1996), and Joseph et al., Cell Cycle, 9(22):4560-4568 (2010)). It follows that the amino acids of the “non-interacting” face p53 peptides described herein includes all amino acids of the p53 transactivation domain except those of the interacting face. In some instances, amino acids of peptides disclosed herein that correspond to amino acids within the interacting face of p53 as disclosed by, e.g., Kussie et al., Science, 274(5289):948-953 (1996) or Joseph et al., Cell Cycle, 9(22):4560-4568 (2010) can be the same or conservative substitutions of the amino acids disclosed by, e.g., Kussie et al., Science, 274(5289):948-953 (1996) and Joseph et al., Cell Cycle, 9(22):4560-4568 (2010). In some instances, the amino acids of the interacting face are E17, F19, S20, L22, W23, L26, P27 and N29 of the amino acid sequence of SEQ ID NO:102. In some instances, the amino acids of the non-interacting face are L14, S15, Q16, T18, D21, K24, L25 and E28 of the amino acid sequence of SEQ ID NO:102. In some instances, in the context of amino acids in the interacting face of the peptides disclosed herein, a conservative amino acid substitution is an amino acid substitution that does not change the structure of the hydrophobic interacting face of the peptide to an extent that significantly impairs the binding interaction. For example, a conservative amino acid substitution is an amino acid substitution that does not reduce (e.g., substantially reduce) binding of the peptide to HDMX. Methods for detecting any reduction in binding can include comparing binding affinity following conservative amino acid substitution, wherein any amino acid substitution that reduces (e.g., substantially reduces) binding are not conservative amino acid substitutions. In some embodiments, substantially reduced binding can include binding that is 10%, 20%, 30%, 40%, 50%, 60%, 70%, 80%, 90%, 95%, 98%, 99%, or 100% less than binding of the unmodified peptide to HDMX. Methods for assessing interaction between a peptide and HDMX are disclosed herein. Methods for identifying the interactive face of a peptide are known in the art (see, e.g., Broglia et al., Protein Sci., 14(10):2668-81, 2005; Hammond et al., J. Pharm. Sci., 98(1):4589-603, 2009; Ng and Yang, J. Phys. Chem. B., 111(50):13886-93, 2007; and Bird et al., PNAS USA, 197:14093, 2010).

In certain instances, the HDMX-selective p53 peptides described herein may contain one or more (e.g., 1, 2, 3, 4, 5, 6, 7, 8, 9, 10) deletions from or additions to the N- and/or C-terminus of the HDMX-selective p53 peptide. For example, the HDMX-selective p53 peptides may be at least 8 amino acids in length but less than 50 (e.g., 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8) amino acids in length. It is to be noted that the triad positions (19, 23, and 26) must be part of the peptide (even if one, two, or three of those residues are substituted by another amino acid than which occurs in SEQ ID NO:102). In certain instances, the HDMX-selective p53 peptides are 8-50 amino acids in length. In certain instances, the HDMX-selective p53 peptides are 8-40 amino acids in length. In certain instances, the HDMX-selective p53 peptides are 8-30 amino acids in length. In certain instances, the HDMX-selective p53 peptides are 8-20 amino acids in length. In certain instances, the HDMX-selective p53 peptides are 8-14 amino acids in length. In certain instances, the HDMX-selective p53 peptides are 8-16 amino acids in length. In certain instances, the HDMX-selective p53 peptides are 14-50 amino acids in length. In certain instances, the HDMX-selective p53 peptides are 14-40 amino acids in length. In certain instances, the HDMX-selective p53 peptides are 14-30 amino acids in length. In certain instances, the HDMX-selective p53 peptides are 14-20 amino acids in length. In certain instances, the HDMX-selective p53 peptides are 16-50 amino acids in length. In certain instances, the HDMX-selective p53 peptides are 16-40 amino acids in length. In certain instances, the HDMX-selective p53 peptides are 16-30 amino acids in length. In certain instances, the HDMX-selective p53 peptides are 16-20 amino acids in length. In certain instances, the HDMX-selective p53 peptides are 14 amino acids in length. In certain instances, the HDMX-selective p53 peptides are 16 amino acids in length. In certain instances in which one or more (e.g., 1, 2, 3, 4, 5, 6, 7, 8, 9, 10) amino acids are deleted from the N- and/or C-terminus of the HDMX-selective p53 peptide, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative, or combinations thereof. In some instances, these HDMX-selective p53 peptide comprises Phe19 and Trp23 of wild type p53.

The disclosure also encompasses HDMX-selective p53 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of any one of SEQ ID NOs:105-116, wherein the peptide retains the substitution(s) at amino acid positions 3 (Q16), 4 (E17), 6 (F19), 9 (L22), 10 (W23), 11 (K24), 13 (L26), 15 (E28), and 16 (N29) in SEQ ID Nos:105-116 relative to SEQ ID NO:99. For example, the disclosure encompasses an HDMX-selective p53 peptide that is at least 14% identical to the amino acid sequence of SEQ ID NO:105, wherein the peptide retains the substitution at position 3 of SEQ ID NO:105 relative to SEQ ID NO:99, i.e., retains the glutamine at position 3 of SEQ ID NO:105. These peptides selectively bind HDMX over HDM2 (e.g., they bind to HDMX with at least 1.5-fold, at least 2-fold, at least 3-fold, at least 4-fold, at least 5-fold, or at least 10-fold greater affinity than to HDM2). In specific instances, the p53 peptide comprises an amino acid sequence that has at least 60%, at least 65%, at least 70%, at least 75%, at least 80%, at least 85%, at least 90% at least 95%, at least 96%, at least 97%, at least 98%, at least 99% sequence identity to the amino acid sequence of any one of SEQ ID NOs: 105-116, wherein the peptide retains the substitution(s) at amino acid positions 3 (Q16), 4 (E17), 6 (F19), 9 (L22), 10 (W23), 11 (K24), 13 (L26), 15 (E28), and 16 (N29) in SEQ ID Nos: 105-116 relative to SEQ ID NO:99. The variability in amino acid sequence of any one of SEQ ID NOs:105-116 can be in interacting-face and/or in the non-interacting face of the peptide. Just about every one of the amino acids on the non-interacting face can be varied. The amino acids on the interacting face can also be varied (e.g., with conservative amino acid substitutions). In some instances, the p53 peptide consists of the amino acid sequence of any one of SEQ ID NOs: 105-116.

The disclosure also encompasses HDMX-selective p53 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of any one of SEQ ID NOs: 117-122, wherein the peptide retains the substitution(s) at amino acid positions 6 (L22), 7 (W23), 8 (K24), 10 (L26), 13 (N29), and 15 (E28) in SEQ ID Nos: 117-122 relative to SEQ ID NO: 101. For example, the disclosure encompasses an HDMX-selective p53 peptide that is at least 14% identical to the amino acid sequence of (SEQ ID NO: 118)-napthalene-(SEQ ID NO:239), wherein the peptide retains the napthalene and substitution at position 3 of SEQ ID NO:239 relative to SEQ ID NO: 101, i.e., retains the naphthalene and the E at position 3 of SEQ ID NO:239. These peptides selectively bind HDMX over HDM2 (e.g., they bind to HDMX with at least 1.5-fold, at least 2-fold, at least 3-fold, at least 4-fold, at least 5-fold, or at least 10-fold greater affinity than to HDM2). In specific instances, the p53 peptide comprises an amino acid sequence that is at least 60%, at least 65%, at least 70%, at least 75%, at least 80%, at least 85%, at least 90%, at least 95%, at least 96%, at least 97%, at least 98%, at least 99% identical to the amino acid sequence of any one of SEQ ID NOs: 117-122, wherein the peptide retains the substitution(s) at amino acid positions 6 (L22), 7 (W23), 8 (K24), 10 (L26), 13 (N29), and 15 (E28) in SEQ ID Nos: 117-122 relative to SEQ ID NO: 101. The variability in amino acid sequence of any one of SEQ ID NOs: 117-122 can be in interacting-face and/or in the non-interacting face of the peptide. Just about every one of the amino acids on the non-interacting face can be varied. The amino acids on the interacting face can also be varied (e.g., with conservative amino acid substitutions). In some instances, the p53 peptide consists of the amino acid sequence of any one of SEQ ID NOs: 117-122.

The disclosure also encompasses HDMX-selective p53 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of SEQ ID NO: 123 or 124, wherein the peptide retains the substitution(s) at amino acid positions 4 (E17) or 13 (L26) in SEQ ID NO: 123 or 124 relative to SEQ ID NO: 100. For example, the disclosure encompasses an HDMX-selective p53 peptide that is at least 14% identical to the amino acid sequence of SEQ ID NO: 124, wherein the peptide retains the substitution at positions 4 and 13 of SEQ ID NO: 124 relative to SEQ ID NO: 100, i.e., retains the E(OMe) at positions 4 and 13 of SEQ ID NO: 124. These peptides selectively bind HDMX over HDM2 (e.g., they bind to HDMX with at least 1.5-fold, at least 2-fold, at least 3-fold, at least 4-fold, at least 5-fold, or at least 10-fold greater affinity than to HDM2). In specific instances, the p53 peptide comprises an amino acid sequence that is at least 60%, at least 65%, at least 70%, at least 75%, at least 80%, at least 85%, at least 90%, at least 95%, at least 96%, at least 97%, at least 98%, at least 99% identical to the amino acid sequence of SEQ ID NO: 123 or 124, wherein the peptide retains the substitution(s) at amino acid positions 4 (E17) or 13 (L26) in SEQ ID NO: 123 or 124 relative to SEQ ID NO: 100. The variability in amino acid sequence of any one of SEQ ID NO: 123 or 124 can be in interacting-face and/or in the non-interacting face of the peptide. Just about every one of the amino acids on the non-interacting face can be varied. The amino acids on the interacting face can also be varied (e.g., with conservative amino acid substitutions). In some instances, the p53 peptide consists of the amino acid sequence of SEQ ID NO: 123 or 124.

Methods for determining percent identity between amino acid sequences are known in the art. For example, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred instance, the length of a reference sequence aligned for comparison purposes is at least 30%, at least 40%, at least 50%, at least 60%, at least 70%, at least 80%, at least 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences is accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997).

In identifying the HDMX-selective structurally-stabilized peptides, HDM2-selective structurally-stabilized and additional HDMX and HDM2 dual-selective structurally-stabilized peptides were also identified. Thus, also provided herein are structurally-stabilized peptides based on the p53-transactivation domain, wherein said structurally-stabilized peptides are selective for HDM2 or are dual-selective for HDMX and HDM2.

### HDM2-Selective Peptides

In certain aspects, the HDM2-selective p53 peptide comprises the amino acid sequence of SAH-p53-4 (SEQ ID NO: 1) or its unstapled precursor (SEQ ID NO:99), with 1 to 10 (e.g., 1, 2, 3, 4, 5, 6, 7, 8, 9, or 10) amino acid substitutions. In some instances, the amino acid substitutions are conservative amino acid substitutions. In some instances, the HDM2-selective p53 peptide comprises a glutamic acid (Glu, E) substituted at the amino acid position corresponding to Leu14 of SEQ ID NO:99, Ser15 of SEQ ID NO:99, or Lys24 of SEQ ID NO:99. In some instances, the HDM2-selective p53 peptide comprises an arginine (Arg, R) at the amino acid position corresponding to Thr18 of SEQ ID NO:99. In some instances, the HDM2-selective p53 peptide comprises an alanine (Ala, A) substituted at the amino acid position corresponding to Ser15 of SEQ ID NO:99, Lys24 of SEQ ID NO:99, or Glu28 of SEQ ID NO:99.

TABLE 2

Exemplary HDM2-selective p53 peptide

| Description | Sequence | SEQ ID NO |
|---|---|---|
| SAH-p53-4 K24E/E28A | LSQETFSDLWELLPAN | 125 |

In certain instances, the HDM2-selective p53 peptides described herein may also contain one or more (e.g., 1, 2, 3, 4, 5, 6, 7, 8, 9, 10) amino acid substitutions (relative to an amino acid sequence set forth SEQ ID NO: 125), e.g., one or more (e.g., 1, 2, 3, 4, 5, 6, 7, 8, 9, 10) conservative and/or non-conservative amino acid substitutions. In certain instances, these substitutions are at the amino acid positions corresponding to positions K24 and/or E28 of SEQ ID NO:99. In certain instances, these substitution(s) are of amino acids on the interacting face of the p53 peptide. In certain instances, these substitution(s) are of amino acids on the non-interacting face of the p53 peptide. In certain instances, these substitutions are of both amino acids on the interacting face of the p53 peptide and on the non-interacting face of the p53 peptide. Just about every one of the amino acids on the non-interacting face of the peptide can be varied. In certain instances, the substituted amino acid(s) are selected from the group consisting of L-Ala, D-Ala, Aib, Ser, a substituted alanine, or a substituted glycine derivative.

In certain instances, the HDM2-selective p53 peptides described herein may contain one or more (e.g., 1, 2, 3, 4, 5, 6, 7, 8, 9, 10) deletions from or additions to the N- and/or C-terminus of the HDM2-selective p53 peptide. For example, the HDM2-selective p53 peptides may be at least 8 amino acids in length but less than 50 (e.g., 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8) amino acids in length. In certain instances, the HDM2-selective p53 peptides are 8-50 amino acids in length. In certain instances, the HDM2-selective p53 peptides are 8-40 amino acids in length. In certain instances, the HDM2-selective p53 peptides are 8-30 amino acids in length. In certain instances, the HDM2-selective p53 peptides are 8-20 amino acids in length. In certain instances, the HDM2-selective p53 peptides are 8-14 amino acids in length. In certain instances, the HDM2-selective p53 peptides are 8-16 amino acids in length. It is to be noted that the triad positions (19, 23, and 26) must be part of the peptide (even if one, two, or three of those residues are substituted by another amino acid than which occurs in SEQ ID NO: 102). In certain instances, the HDM2-selective p53 peptides are 14-50 amino acids in length. In certain instances, the HDM2-selective p53 peptides are 14-40 amino acids in length. In certain instances, the HDM2-selective p53 peptides are 14-30 amino acids in length. In certain instances, the HDM2-selective p53 peptides are 14-20 amino acids in length. In certain instances, the HDM2-selective p53 peptides are 16-50 amino acids in length. In certain instances, the HDM2-selective p53 peptides are 16-40 amino acids in length. In certain instances, the HDM2-selective p53 peptides are 16-30 amino acids in length. In certain instances, the HDM2-selective p53 peptides are 16-20 amino acids in length. In certain instances, the HDM2-selective p53 peptides are 14 amino acids in length. In certain instances, the HDM2-selective p53 peptides are 16 amino acids in length. In certain instances in which one or more (e.g., 1, 2, 3, 4, 5, 6, 7, 8, 9, 10) amino acids are deleted from the N- and/or C-terminus of the HDM2-selective p53 peptide, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative, or combinations thereof.

The disclosure also encompasses HDM2-selective p53 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of SEQ ID NO: 125, wherein the peptide retains the substitution(s) at amino acid positions 11 (K24) and/or 15 (E28) in SEQ ID NO: 125 relative to SEQ ID NO:99. For example, the disclosure encompasses an HDM2-selective p53 peptide that is at least 14% identical to the amino acid sequence of SEQ ID NO: 125, wherein the peptide retains the substitution at position 1 of SEQ ID NO: 125 relative to SEQ ID NO:99, i.e., retains the glutamic acid at position 1 of SEQ ID NO: 125. These peptides selectively bind HDM2 over HDMX (e.g., they bind to HDM2 with at least 1.5-fold, at least 2-fold, at least 3-fold, at least 4-fold, at least 5-fold, or at least 10-fold greater affinity than to HDMX). The variability in amino acid sequence of SEQ ID NO: 125 can be in interacting-face and/or in the non-interacting face of the peptide. Just about every one of the amino acids on the non-interacting face can be varied. The amino acids on the interacting face can also be varied (e.g., with conservative amino acid substitutions). In some instances, the p53 peptide consists of the amino acid sequence of SEQ ID NO: 125.

In some instances, an HDM2-selective peptide binds to both HDM2 and HDMX. In some instances, an HDM2-selective peptide has an at least 0.2-fold higher, at least 0.5-fold higher, at least 1-fold higher, at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity to HDM2 than to HDMX as determined by, e.g., isothermal calorimetry or fluorescence polarization analyses.

### HDMX/HDM2 Dual-Selective Peptides

In certain aspects, the HDMX/HDM2 dual-selective p53 peptide comprises the amino acid sequence of SAH-p53-4 (SEQ ID NO: 1) or its unstapled precursor (SEQ ID NO:99), with 1 to 10 (e.g., 1, 2, 3, 4, 5, 6, 7, 8, 9, or 10) amino acid substitutions. In some instances, the amino acid substitutions are conservative amino acid substitutions.

In some instances, the HDM2/HDMX dual-selective (dual-binding) stabilized peptide comprises the amino acid sequence set forth in one of SEQ ID NOs: 9, 22, 26, 47, 60, 77, 79, and 81 and variants thereof. Variants include sequences with 1 to 10 (e.g., 1, 2, 3, 4, 5, 6, 7, 8, 9, 10) substitutions relative to SEQ ID NOs: 9, 22, 26, 47, 60, 77, 79, and 81. These variants bind both HDM2 and HDMX.

TABLE 3

Exemplary HDMX/HDM2 dual-selective p53 peptides

| Description | Sequence | SEQ ID NO |
|---|---|---|
| SAH-p53-4 K24E/E28A | LSQETFSDLWELLPAN | 125 |
| SAH-p53-4 L14(A/R/E) | X₁SQETFSDLWKLLPEN, wherein X₁ is A, R, or E | 126 |
| SAH-p53-4 S15(A/R/E) | LX₁QETFSDLWKLLPEN, wherein X₁ is A, R, or E | 127 |
| SAH-p53-4 Q16(A/R) | LSX₁ETFSDLWKLLPEN, wherein X₁ is A or R | 128 |
| SAH-p53-4 E17R | LSQRTFSDLWKLLPEN | 129 |
| SAH-p53-4 T18 (A/R/E) | LSQEX₁FSDLWKLLPEN, wherein X₁ is A, R, or E | 130 |
| SAH-p53-4 D21(R/E) | LSQETFSX₁LWKLLPEN, wherein X₁ is R or E | 131 |
| SAH-p53-4 L22(A/E) | LSQETFSDX₁WKLLPEN, wherein X₁ is A or E | 132 |
| SAH-p53-4 K24(A/R) | LSQETFSDLW X₁LLPEN, wherein X₁ is A or R | 133 |
| SAH-p53-4 L25(A/R/E) | LSQETFSDLWKX₁LPEN, wherein X₁ is A, R, or E | 134 |
| SAH-p53-4 E28(A/R) | LSQETFSDLWKLLPX₁N, wherein X1 is A or R | 135 |
| SAH-p53-4 N29(A/E) | LSQETFSDLWKLLPEX₁, wherein X₁ is A or E | 136 |
| ATSP-7041 A24E | LTF8EYWEQBXSAA | 137 |
| ATSP-7041 B26(F/I/L/b) | LTFEEYWAQX₁TSAA, wherein X₁ is a F, I, L, or norleucine | 138 |
| ATSP-7041 S28A | LTF8EYWAQBXAAA | 139 |
| ATSP-7041 A24E/S28E | LTF8EYWEQBXEAA | 140 |

In certain instances, the HDMX/HDM2 dual-selective p53 peptides described herein may also contain one or more (e.g., 1, 2, 3, 4, 5, 6, 7, 8, 9, 10) amino acid substitutions (relative to an amino acid sequence set forth in any one of SEQ ID NOs: 125-140), e.g., one or more (e.g., 1, 2, 3, 4, 5, 6, 7, 8, 9, 10) conservative and/or non-conservative amino acid substitutions. In certain instances, these substitutions are at the amino acid positions corresponding to positions L14, S15, Q16, E17, T18, D21, L22, K24, L25, E28, or N29 of SEQ ID NO:99. In certain instances, these substitutions are at the amino acid positions corresponding to positions A24, B26, and S28 of SEQ ID NO:101.

In certain instances, these substitution(s) are of amino acids on the interacting face of the p53 peptide. In certain instances, these substitution(s) are of amino acids on the non-interacting face of the p53 peptide. In certain instances, these substitutions are of both amino acids on the interacting face of the p53 peptide and on the non-interacting face of the p53 peptide. Just about every one of the amino acids on the non-interacting face of the peptide can be varied. In certain instances, the substituted amino acid(s) are selected from the group consisting of L-Ala, D-Ala, Aib, Ser, a substituted alanine, or a substituted glycine derivative.

In certain instances, the HDMX/HDM2 dual-selective p53 peptides described herein may contain one or more (e.g., 1, 2, 3, 4, 5, 6, 7, 8, 9, 10) deletions from or additions to the N- and/or C-terminus of the HDM2-selective p53 peptide. For example, the HDM2-selective p53 peptides may be at least 8 amino acids in length but less than 50 (e.g., 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8) amino acids in length. It is to be noted that the triad positions (19, 23, and 26) must be part of the peptide (even if one, two, or three of those residues are substituted by another amino acid than which occurs in SEQ ID NO: 102). In certain instances, the HDMX/HDM2 dual-selective p53 peptides are 8-50 amino acids in length. In certain instances, the HDMX/HDM2 dual-selective p53 peptides are 8-40 amino acids in length. In certain instances, the HDMX/HDM2 dual-selective p53 peptides are 8-30 amino acids in length. In certain instances, the HDMX/HDM2 dual-selective p53 peptides are 8-20 amino acids in length. In certain instances, the HDMX/HDM2 dual-selective p53 peptides are 8-16 amino acids in length. In certain instances, the HDMX/HDM2 dual-selective p53 peptides are 8-14 amino acids in length. In certain instances, the HDMX/HDM2 dual-selective p53 peptides are 14-50 amino acids in length. In certain instances, the HDMX/HDM2 dual-selective p53 peptides are 14-40 amino acids in length. In certain instances, the HDMX/HDM2 dual-selective p53 peptides are 14-30 amino acids in length. In certain instances, the HDMX/HDM2 dual-selective p53 peptides are 14-20 amino acids in length. In certain instances, the HDMX/HDM2 dual-selective p53 peptides are 16-50 amino acids in length. In certain instances, the HDMX/HDM2 dual-selective p53 peptides are 16-40 amino acids in length. In certain instances, the HDMX/HDM2 dual-selective p53 peptides are 16-30 amino acids in length. In certain instances, the HDMX/HDM2 dual-selective p53 peptides are 16-20 amino acids in length. In certain instances, the HDMX/HDM2 dual-selective p53 peptides are 14 amino acids in length. In certain instances, the HDMX/HDM2 dual-selective p53 peptides are 16 amino acids in length. In certain instances in which one or more (e.g., 1, 2, 3, 4, 5, 6, 7, 8, 9, 10) amino acids are deleted from the N- and/or C-terminus of the HDMX/HDM2 dual-selective p53 peptide, these removed amino acids can be replaced with 1-6 (e.g., 1, 2, 3, 4, 5, or 6) amino acids selected from the group consisting of L-Ala, D-Ala, Aib, Sar, Ser, a substituted alanine, or a substituted glycine derivative, or combinations thereof.

The disclosure also encompasses HDMX/HDM2 dual-selective p53 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of any one of SEQ ID NOs: 130-139, wherein the peptide retains the substitution(s) at amino acid positions 1 (L14), 2 (S15), 3 (Q16), 4 (E17), 5 (T18), 8 (D21), 9 (L22), 11 (K24), 12 (L25), 15 (E28), and 16 (N29) in SEQ ID NOs: 126-136 relative to SEQ ID NO:99. For example, the disclosure encompasses an HDMX/HDM2 dual-selective p53 peptide that is at least 14% identical to the amino acid sequence of SEQ ID NO: 126, wherein the peptide retains the substitution at position 1 of SEQ ID NO: 126 relative to SEQ ID NO:99, i.e., retains the alanine or arginine at position 1 of SEQ ID NO: 126. The variability in amino acid sequence of any one of SEQ ID NOs: 126-136 can be in interacting-face and/or in the non-interacting face of the peptide. Just about every one of the amino acids on the non-interacting face can be varied. The amino acids on the interacting face can also be varied (e.g., with conservative amino acid substitutions). In some instances, the p53 peptide consists of the amino acid sequence of any one of SEQ ID NOs: 126-136.

The disclosure also encompasses HDMX/HDM2 dual-selective p53 peptides that are at least 14% (e.g., at least 14% to 50%, at least 14% to 45%, at least 14% to 40%, at least 14% to 35%, at least 14% to 30%, at least 14% to 25%, at least 14% to 20%, at least 20% to 50%, at least 20% to 45%, at least 20% to 40%, at least 20% to 35%, at least 20% to 30%, at least 20% to 25%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) identical to the amino acid sequence of any one of SEQ ID NOs: 137-140, wherein the peptide retains the substitution(s) at amino acid positions 8 (A24), 10 (B26), and 12 (S28) in SEQ ID Nos: 137-140 relative to SEQ ID NO:101. For example, the disclosure encompasses an HDMX/HDM2 dual-selective p53 peptide that is at least 14% identical to the amino acid sequence of SEQ ID NO: 137, wherein the peptide retains the substitution at position 1 of SEQ ID NO: 137 relative to SEQ ID NO:99, i.e., retains the glutamic acid at position 8 of SEQ ID NO: 137. The variability in amino acid sequence of any one of SEQ ID NOs: 137-140 can be in interacting-face and/or in the non-interacting face of the peptide. Just about every one of the amino acids on the non-interacting face can be varied. The amino acids on the interacting face can also be varied (e.g., with conservative amino acid substitutions). In some instances, the p53 peptide consists of the amino acid sequence of any one of SEQ ID NOs: 137-140.

The p53 peptides described herein can be optimized for therapeutic use. For example, if any of the above-described p53 peptides cause undesired membrane disruption (cell lysis of, e.g., red blood cells), the peptides can be optimized by lowering the overall peptide hydrophobicity. This can for example be achieved by substituting especially hydrophobic residues with an amino acid with lower hydrophobicity (e.g., lysine or glutamate). Membrane disruption can also be lowered by reducing the overall positive charge of the peptide. This can be accomplished by substituting basic residues with uncharged or acidic residues. In certain instances, both the overall peptide hydrophobicity and the overall positive charge of the peptide are lowered.

### Structurally-Stabilized P53 Peptides

A peptide helix is an important mediator of key protein-protein interactions that regulate many important biological processes such as apoptosis; however, when such a helix is taken out of its context within a protein and prepared in isolation, it usually adopts a random coil conformation, leading to a drastic reduction in biological activity and thus diminished therapeutic potential. The present disclosure provides structurally-stabilized p53 peptides (e.g., HDMX-selective peptides). In some instances, the structurally-stabilized p53 peptides described herein retain two members of the triad of amino acids that are essential for binding to HDM2 and HDMX: amino acids Phe19 (F19), Trp23 (W23) (numbered according to the amino acid sequence set forth in SEQ ID NO: 102). In some instances, the structurally-stabilized p53 peptides are HDM2-selective. In some instances, the structurally-stabilized p53 peptides are HDMX-selective. In some instances, HDMX-selective structurally-stabilized p53 peptides include one or more (1, 2, or 3) modifications of the p53 triad Phe19 (F19), Trp23 (W23), and Leu26 (L26) (numbered according to the amino acid sequence set forth in SEQ ID NO: 102) converts p53 peptides into HDMX-selective peptides. In some instances, the structurally-stabilized p53 peptides are selective for both HDM2 and HDMX. The present disclosure includes structurally-stabilized p53 peptides (such as structurally-stabilized versions of the p53 peptide described above and structurally-stabilized variants of SAH-p53-4, SAH-p53-8, and ATSP-7041 and their unstapled precursors described above) comprising at least two (e.g., 2, 3, 4, 5, 6) modified amino acids joined by an internal (intramolecular) cross-link (e.g., a staple or stitch). The structural-stabilization may be by, e.g., “stapling” the peptide. In some cases, the staple is a hydrocarbon staple. Stabilized peptides as described herein include stapled peptides and stitched peptides as well as peptides containing multiple stitches, multiple staples or a mix of staples and stitches, or any other chemical strategies for structural reinforcement (see. e.g., Balaram P. Cur. Opin. Struct. Biol. 1992;2:845; Kemp DS, et al., J. Am. Chem. Soc. 1996;118:4240; Orner BP, et al., J. Am. Chem. Soc. 2001; 123:5382; Chin JW, etal., Int. Ed. 2001;40:3806; Chapman RN, et al., J. Am. Chem. Soc. 2004;126:12252; Home WS, et al., Chem., Int. Ed. 2008;47:2853; Madden et al., Chem Commun (Camb). 2009 Oct 7; (37): 5588-5590; Lau et al., Chem. Soc. Rev., 2015,44:91-102; and Gunnoo et al., Org. Biomol. Chem., 2016,14:8002-8013; all of which are incorporated by reference herein in its entirety).

In certain instances, one or more of the p53 peptides described herein can be structurally-stabilized by peptide stapling (see, e.g., Walensky, J. Med. Chem., 57:6275-6288 (2014), the contents of which are incorporated by reference herein in its entirety). A peptide is “structurally-stabilized” in that it maintains its native secondary structure. For example, stapling allows a peptide, predisposed to have an α-helical secondary structure, to maintain its native α-helical conformation. This secondary structure increases resistance of the peptide to proteolytic cleavage and heat, and also may increase target binding affinity, hydrophobicity, and cell permeability. Accordingly, the stapled (cross-linked) peptides described herein have improved biological activity relative to a corresponding non-stapled (un-cross-linked) peptide.

“Peptide stapling” is a term coined from a synthetic methodology wherein two olefin-containing side-chains (e.g., cross-linkable side chains) present in a peptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the j oining of two (e.g., at least one pair of) double bond-containing side-chains, triple bond-containing side-chains, or double bond-containing and triple bond-containing side chain, which may be present in a peptide chain, using any number of reaction conditions and/or catalysts to facilitate such a reaction, to provide a singly “stapled” peptide. The term “multiply stapled” peptides refers to those peptides containing more than one individual staple, and may contain two, three, or more independent staples of various spacing. Additionally, the term “peptide stitching,” as used herein, refers to multiple and tandem “stapling” events in a single peptide chain to provide a “stitched” (e.g., tandem or multiply stapled) peptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced.

In certain instances, one or more of the p53 peptides described herein can be structurally-stabilized. In some instances, the p53 peptides of this disclosure are structurally-stabilized by a hydrocarbon staple or stitch, a lactam staple or stitch; a UV-cycloaddition staple or stitch; an oxime staple or stitch; a thioether staple or stitch; a double-click staple or stitch; a bis-lactam staple or stitch; a bis-arylation staple or stitch; or a combination of any two or more thereof. In some instances, the p53 peptides of this disclosure are structurally-stabilized by a hydrocarbon staple. In some instances, the p53 peptides of this disclosure are structurally-stabilized by a hydrocarbon stitch.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄ (SEQ ID NO:241), wherein X₁ is L or a conservative amino acid substitution thereof; X₂ is T or a conservative amino acid substitution thereof; X₃ is F or a conservative amino acid substitution thereof; X₄ is a stapling amino acid; X₅ is E or a conservative amino acid substitution thereof; X₆ is Y or a conservative amino acid substitution thereof; X₇ is W or a conservative amino acid substitution thereof; Xs is A or a conservative amino acid substitution thereof; X₉ is Q or a conservative amino acid substitution thereof; X₁₀ is any amino acid except phenylalanine, isoleucine, norleucine, and leucine; X₁₁ is a stapling amino acid; X₁₂ is S or a conservative amino acid substitution thereof; X₁₃ is A or a conservative amino acid substitution thereof; and X₁₄ is A or a conservative amino acid substitution thereof. In some instances of SEQ ID NO:241, X₁₀ is alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C). In some instances, X₁₀ is glutamate (E), 5-fluoronorvaline (f), or O-methylated glutamic acid (E(OMe)). In some instances, X₄ is (R)-2-(4′-pentenyl)Alanine and X₁₁ is (S)-2-(7′-octenyl)Alanine. In some instances, X₄ is (R)-2-(7′-octenyl)Alanine and X₁₁ is (S)-2-(4′-pentenyl)Alanine. In some instances, the amino acids of the interacting face are X₁, X₃, X₄, X₆, X₇, X₁₀, and X₁₁. In some cases, the amino acids of the interacting face are not substituted. In some cases 1 to 4, 1 to 3, 1 to 2, or 1 amino acids of the interacting face are substituted (e.g., conservative amino acid substitutions). In some instances, the amino acids of the non-interacting face are X₂, X₅, X₈, X₉, X₁₂, X₁₃, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄ (SEQ ID NO: 144), wherein X₁ is L or a conservative amino acid substitution thereof; X₂ is T or a conservative amino acid substitution thereof; X₃ is F or a conservative amino acid substitution thereof; X₄ is a stapling amino acid; X₅ is E or a conservative amino acid substitution thereof; X₆ is Y or a conservative amino acid substitution thereof; X₇ is W or a conservative amino acid substitution thereof; Xs is A or a conservative amino acid substitution thereof; X₉ is Q or a conservative amino acid substitution thereof; X₁₀ is a charged amino acid, alanine, a hydrophobic amino acid less hydrophobic than alanine, or a hydrophilic amino acid; X₁₁ is a stapling amino acid; X₁₂ is S or a conservative amino acid substitution thereof; X₁₃ is A or a conservative amino acid substitution thereof; and X₁₄ is A or a conservative amino acid substitution thereof. In some instances of SEQ ID NO: 144, X₁₀ is alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C). In some instances, X₁₀ is glutamate (E), 5-fluoronorvaline (f), or O-methylated glutamic acid (E(OMe)). In some instances, X₄ is (R)-2-(4′-pentenyl)Alanine and X₁₁ is (S)-2-(7′-octenyl)Alanine. In some instances, X₄ is (R)-2-(7′-octenyl)Alanine and X₁₁ is (S)-2-(4′-pentenyl)Alanine. In some instances, the amino acids of the interacting face are X₁, X₃, X₄, X₆, X₇, X₁₀, and X₁₁. In some cases, the amino acids of the interacting face are not substituted. In some cases 1 to 4, 1 to 3, 1 to 2, or 1 amino acids of the interacting face are substituted (e.g., conservative amino acid substitutions). In some instances, the amino acids of the non-interacting face are X₂, X₅, X₈, X₉, X₁₂, X₁₃, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄ (SEQ ID NO: 145), wherein X₁ is L or a conservative amino acid substitution thereof; X₂ is T or a conservative amino acid substitution thereof; X₃ is charged amino acid or alanine; X₄ is a stapling amino acid; X₅ is E or a conservative amino acid substitution thereof; X₆ is Y or a conservative amino acid substitution thereof; X₇ is W or a conservative amino acid substitution thereof; Xs is A or a conservative amino acid substitution thereof; X₉ is Q or a conservative amino acid substitution thereof; X₁₀ is L or cyclobutyl alanine; X₁₁ is a stapling amino acid; X₁₂ is S or a conservative amino acid substitution thereof; X₁₃ is A or a conservative amino acid substitution thereof; and X₁₄ is A or a conservative amino acid substitution thereof. In some instances of SEQ ID NO: 145, X₃ is alanine (A), arginine (R), or glutamate (E). In some instances, X₄ is (S)-2-(4′-pentenyl)Alanine and X₁₁ is (R)-2-(7′-octenyl)Alanine. In some instances, X₄ is (R)-2-(7′-octenyl)Alanine and X₁₁ is (S)-2-(4′-pentenyl)Alanine. In some instances, the amino acids of the interacting face are X₁, X₃, X₄, X₆, X₇, X₁₀, and X₁₁. In some cases, the amino acids of the interacting face are not substituted. In some cases 1 to 4, 1 to 3, 1 to 2, or 1 amino acids of the interacting face are substituted (e.g., conservative amino acid substitutions). In some instances, the amino acids of the non-interacting face are X₂, X₅, X₈, X₉, X₁₂, X₁₃, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄ (SEQ ID NO: 146, wherein X₁ is L or a conservative amino acid substitution thereof; X₂ is T or a conservative amino acid substitution thereof; X₃ is F or a conservative amino acid substitution thereof; X₄ is a stapling amino acid; X₅ is E or a conservative amino acid substitution thereof; X₆ is Y or a conservative amino acid substitution thereof; X₇ is a charged amino acid, alanine, or an aromatic amino acid other than W; Xs is A or a conservative amino acid substitution thereof; X₉ is Q or a conservative amino acid substitution thereof; X₁₀ is L or cyclobutyl alanine; X₁₁ is a stapling amino acid; X₁₂ is S or a conservative amino acid substitution thereof; X₁₃ is A or a conservative amino acid substitution thereof; and X₁₄ is A or a conservative amino acid substitution thereof. In some instances of SEQ ID NO: 146, X₇ is naphthalene, alanine (A), arginine (R), or glutamate (E). In some instances, X₄ is (S)-2-(4′-pentenyl)Alanine and X₁₁ is (R)-2-(7′-octenyl)Alanine. In some instances, X₄ is (R)-2-(7′-octenyl)Alanine and X₁₁ is (S)-2-(4′-pentenyl)Alanine. In some instances, the amino acids of the interacting face are X₁, X₃, X₄, X₆, X₇, X₁₀, and X₁₁. In some cases, the amino acids of the interacting face are not substituted. In some cases 1 to 4, 1 to 3, 1 to 2, or 1 amino acids of the interacting face are substituted (e.g., conservative amino acid substitutions). In some instances, the amino acids of the non-interacting face are X₂, X₅, X₈, X₉, X₁₂, X₁₃, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄X₁₅X₁₆ (SEQ ID NO:242, wherein X₁ is L or a conservative amino acid substitution thereof or Q or a conservative amino acid substitution thereof; X₂ is S or a conservative amino acid substitution thereof; X₃ is Q or a conservative amino acid substitution thereof; X₄ is E or a conservative amino acid substitution thereof or Q or a conservative amino acid substitution thereof; X₅ is T or a conservative amino acid substitution thereof; X₆ is F or a conservative amino acid substitution thereof; X₇ is a stapling amino acid; X₈ is D or a conservative amino acid substitution thereof or N or a conservative amino acid substitution thereof; X₉ is L or a conservative amino acid substitution thereof; X₁₀ is W or a conservative amino acid substitution thereof; X₁₁ is K or a conservative amino acid substitution thereof or R or a conservative amino acid substitution thereof; X₁₂ is L or a conservative amino acid substitution thereof; X₁₃ is any amino acid except phenylalanine, isoluecine, norleucine, and leucine; X₁₄ is a stapling amino acid; X₁₅ is E or a conservative amino acid substitution thereof or Q or a conservative amino acid substitution thereof; and X₁₆ is N or a conservative amino acid substitution thereof. In some instances of SEQ ID NO:242, X₁₃ is alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C). In some instances, X₁₃ is glutamate (E), 5-fluoronorvaline (f), or O-methylated glutamic acid (E(OMe)). In some instances, X₇ is (S)-2-(4′-pentenyl)Alanine and X₁₄ is (R)-2-(7′-octenyl)Alanine. In some instances, X₇ is (R)-2-(7′-octenyl)Alanine and X₁₄ is (S)-2-(4′-pentenyl)Alanine. In some instances, the amino acids of the interacting face are X₄, X₆, X₇, X₉, X₁₀, X₁₂, X₁₅, and X1₆. In some cases, the amino acids of the interacting face are not substituted. In some cases 1 to 5, 1 to 4, 1 to 3, 1 to 2, or 1 amino acids of the interacting face are substituted (e.g., conservative amino acid substitutions). In some instances, the amino acids of the non-interacting face are X₁, X₂, X₃, X₅, X₈, X₁₁, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄X₁₅X₁₆ (SEQ ID NO:240, wherein X₁ is L or a conservative amino acid substitution thereof or Q or a conservative amino acid substitution thereof; X₂ is S or a conservative amino acid substitution thereof; X₃ is Q or a conservative amino acid substitution thereof; X₄ is E or a conservative amino acid substitution thereof or Q or a conservative amino acid substitution thereof; X₅ is T or a conservative amino acid substitution thereof; X₆ is F or a conservative amino acid substitution thereof; X₇ is a stapling amino acid; X₈ is D or a conservative amino acid substitution thereof or N or a conservative amino acid substitution thereof; X₉ is L or a conservative amino acid substitution thereof; X₁₀ is W or a conservative amino acid substitution thereof; X₁₁ is K or a conservative amino acid substitution thereof or R or a conservative amino acid substitution thereof; X₁₂ is L or a conservative amino acid substitution thereof; X₁₃ is a charged amino acid, alanine, a hydrophobic amino acid less hydrophobic than alanine, or a hydrophilic amino acid; X₁₄ is a stapling amino acid; X₁₅ is E or a conservative amino acid substitution thereof or Q or a conservative amino acid substitution thereof; and X₁₆ is N or a conservative amino acid substitution thereof. In some instances of SEQ ID NO:240, X₁₃ is alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C). In some instances, X₁₃ is glutamate (E), 5-fluoronorvaline (f), or O-methylated glutamic acid (E(OMe)). In some instances, X₇ is (S)-2-(4′-pentenyl)Alanine and X₁₄ is (R)-2-(7′-octenyl)Alanine. In some instances, X₇ is (R)-2-(7′-octenyl)Alanine and X₁₄ is (S)-2-(4′-pentenyl)Alanine. In some instances, the amino acids of the interacting face are X₄, X₆, X₇, X₉, X₁₀, X₁₂, X₁₅, and X1₆. In some cases, the amino acids of the interacting face are not substituted. In some cases 1 to 5, 1 to 4, 1 to 3, 1 to 2, or 1 amino acids of the interacting face are substituted (e.g., conservative amino acid substitutions). In some instances, the amino acids of the non-interacting face are X₁, X₂, X₃, X₅, X₈, X₁₁, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄X₁₅X₁₆ (SEQ ID NO: 148, wherein X₁ is L or a conservative amino acid substitution thereof or Q or a conservative amino acid substitution thereof; X₂ is S or a conservative amino acid substitution thereof; X₃ is Q or a conservative amino acid substitution thereof; X₄ is E or a conservative amino acid substitution thereof or Q or a conservative amino acid substitution thereof; X₅ is T or a conservative amino acid substitution thereof; X₆ is charged amino acid or alanine; X₇ is a stapling amino acid; X₈ is D or a conservative amino acid substitution thereof or N or a conservative amino acid substitution thereof; X₉ is L or a conservative amino acid substitution thereof; X₁₀ is W or a conservative amino acid substitution thereof; X₁₁ is K or a conservative amino acid substitution thereof or R or a conservative amino acid substitution thereof; X₁₂ is L or a conservative amino acid substitution thereof; X₁₃ is L or a conservative amino acid substitution thereof; X₁₄ is a stapling amino acid; X₁₅ is E or a conservative amino acid substitution thereof or Q or a conservative amino acid substitution thereof; and X₁₆ is N or a conservative amino acid substitution thereof. In some instances of SEQ ID NO: 148, X₆ is alanine (A), arginine (R), or glutamate (E). In some instances, X₇ is (S)-2-(4′-pentenyl)Alanine and X₁₄ is (R)-2-(7′-octenyl)Alanine. In some instances, X₇ is (R)-2-(7′-octenyl)Alanine and X₁₄ is (S)-2-(4′-pentenyl)Alanine. In some instances, the amino acids of the interacting face are X₄, X₆, X₇, X₉, X₁₀, X₁₂, X₁₃, X₁₅, and X₁₆. In some cases, the amino acids of the interacting face are not substituted. In some cases 1 to 5, 1 to 4, 1 to 3, 1 to 2, or 1 amino acids of the interacting face are substituted (e.g., conservative amino acid substitutions). In some instances, the amino acids of the non-interacting face are X₁, X₂, X₃, X₅, X₈, X₁₁, and X₁₄. In some cases, 1 to 7, 1 to 6, 1 to 5, 1 to 4, 1 to 3, 1 to 2, or 1 of the amino acids of the non-interacting face are substituted.

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises the sequence: X₁X₂X₃X₄X₅X₆X₇X₈X₉X₁₀X₁₁X₁₂X₁₃X₁₄X₁₅X₁₆ (SEQ ID NO: 149, wherein X₁ is L or a conservative amino acid substitution thereof or Q or a conservative amino acid substitution thereof; X₂ is S or a conservative amino acid substitution thereof; X₃ is Q or a conservative amino acid substitution thereof; X₄ is E or a conservative amino acid substitution thereof or Q or a conservative amino acid substitution thereof; X₅ is T or a conservative amino acid substitution thereof; X₆ is F or a conservative amino acid substitution thereof; X₇ is a stapling amino acid; X₈ is D or a conservative amino acid substitution thereof or N or a conservative amino acid substitution thereof; X₉ is L or a conservative amino acid substitution thereof; X₁₀ is a charged amino acid, alanine, or an aromatic amino acid other than W; X₁₁ is K or a conservative amino acid substitution thereof or R or a conservative amino acid substitution thereof; X₁₂ is L or a conservative amino acid substitution thereof; X₁₃ is L or a conservative amino acid substitution thereof; X₁₄ is a stapling amino acid; X₁₅ is E or a conservative amino acid substitution thereof or Q or a conservative amino acid substitution thereof; and X₁₆ is N or a conservative amino acid substitution thereof. In some instances of SEQ ID NO: 149, X₁₀ is naphthalene, alanine (A), arginine (R), or glutamate (E). In some instances, X₇ is (R)-2-(4′-pentenyl)Alanine and X₁₄ is (S)-2-(7′-octenyl)Alanine. In some instances, X₇ is (R)-2-(7′-octenyl)Alanine and X₁₄ is (S)-2-(4′-pentenyl)Alanine. In some instances, the amino acids of the interacting face are X₄, X₆, X₇, X₉, X₁₀, X₁₂, X₁₃, X₁₅, and X₁₆. In some cases, the amino acids of the interacting face are not substituted. In some cases 1 to 5, 1 to 4, 1 to 3, 1 to 2, or 1 amino acids of the interacting face are substituted (e.g., conservative amino acid substitutions). In some instances, the amino acids of the non-interacting face are X₁, X₂, X₃, X₅, X₈, X₁₁, and X₁₄. In some cases, 1 to 7, 1 to 6, 1 to 5, 1 to 4, 1 to 3, 1 to 2, or 1 of the amino acids of the non-interacting face are substituted.

In some instances, the structurally-stabilized peptide selectively binds to HDMX and comprises the amino acid sequence LTFEEYWAQBTSAA (SEQ ID NO: 101), comprising 2 to 10 (2, 3, 4, 5, 6, 7, 8, 9, or 10) amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with any amino acid other than phenylalanine, isoleucine, norleucine, and leucine, (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid, alanine, or an aromatic amino acid, wherein the structurally-stabilized peptide is stapled or stitched. In some instances, the structurally-stabilized peptide comprises a phenylalanine at the amino acid corresponding to F19 of SEQ ID NO: 102. In some instances, the structurally-stabilized peptide comprises a tryptophan at the amino acid corresponding to W23 of SEQ ID NO: 102.

In some instances, the structurally-stabilized peptide selectively binds to HDMX and comprises the amino acid sequence LTFEEYWAQBTSAA (SEQ ID NO: 101), comprising 2 to 10 (2, 3, 4, 5, 6, 7, 8, 9, or 10) amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with a charged amino acid, alanine, a hydrophobic amino acid less hydrophobic than leucine, or a hydrophilic amino acid, (ii) substitution of the amino acid corresponding to F 19 of SEQ ID NO: 102 with a charged amino acid or alanine, or (iii) substitution of the amino acid corresponding to W23 of SEQ ID NO: 102 with a charged amino acid, alanine, or an aromatic amino acid, wherein the structurally-stabilized peptide is stapled or stitched. In some instances, the structurally-stabilized peptide comprises a phenylalanine at the amino acid corresponding to F 19 of SEQ ID NO: 102. In some instances, the structurally-stabilized peptide comprises a tryptophan at the amino acid corresponding to W23 of SEQ ID NO: 102.

In some instances, the structurally-stabilized peptide selectively binds to HDMX and comprises the amino acid sequence LSQETFSDLWKLLPEN (SEQ ID NO:99), comprising 2 to 10 (2, 3, 4, 5, 6, 7, 8, 9, or 10) amino acid substitutions relative to the amino acid sequence of SEQ ID NO:99, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with any amino acid other than phenylalanine, isoleucine, norleucine, and leucine, (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid, alanine, or an aromatic amino acid, wherein the structurally-stabilized peptide is stapled or stitched. In some instances, the structurally-stabilized peptide comprises a phenylalanine at the amino acid corresponding to F19 of SEQ ID NO: 102. In some instances, the structurally-stabilized peptide comprises a tryptophan at the amino acid corresponding to W23 of SEQ ID NO: 102.

In some instances, the structurally-stabilized peptide selectively binds to HDMX and comprises the amino acid sequence LSQETFSDLWKLLPEN (SEQ ID NO:99), comprising 2 to 10 (2, 3, 4, 5, 6, 7, 8, 9, or 10) amino acid substitutions relative to the amino acid sequence of SEQ ID NO:99, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with any amino acid other than phenylalanine, isoleucine, norleucine, and leucine (e.g., a charged amino acid (e.g., arginine or glutamic acid), alanine, a hydrophobic amino acid less hydrophobic than leucine, or a hydrophilic amino acid), (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid, alanine, or an aromatic amino acid, wherein the structurally-stabilized peptide is stapled or stitched. In some instances, the structurally-stabilized peptide comprises a phenylalanine at the amino acid corresponding to F19 of SEQ ID NO: 102. In some instances, the structurally-stabilized peptide comprises a tryptophan at the amino acid corresponding to W23 of SEQ ID NO: 102.

In some instances, the structurally-stabilized peptide selectively binds to HDMX and comprises the amino acid sequence QSQQTFSNLWRLLPQN (SEQ ID NO: 100), comprising 2 to 10 (2, 3, 4, 5, 6, 7, 8, 9, or 10) amino acid substitutions relative to the amino acid sequence of SEQ ID NO: 100, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with any amino acid other than phenylalanine, isoleucine, norleucine, and leucine, (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid, alanine, or an aromatic amino acid, wherein the structurally-stabilized peptide is stapled or stitched. In some instances, the structurally-stabilized peptide comprises a phenylalanine at the amino acid corresponding to F19 of SEQ ID NO: 102. In some instances, the structurally-stabilized peptide comprises a tryptophan at the amino acid corresponding to W23 of SEQ ID NO: 102.

In some instances, the structurally-stabilized peptide selectively binds to HDMX and comprises the amino acid sequence QSQQTFSNLWRLLPQN (SEQ ID NO: 100), comprising 2 to 10 (2, 3, 4, 5, 6, 7, 8, 9, or 10) amino acid substitutions relative to the amino acid sequence of SEQ ID NO: 100, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with any amino acid other than phenylalanine, isoleucine, norleucine, and leucine (e.g., a charged amino acid (e.g., arginine or glutamic acid), alanine, a hydrophobic amino acid less hydrophobic than leucine, or a hydrophilic amino acid), (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid, alanine, or an aromatic amino acid, wherein the structurally-stabilized peptide is stapled or stitched. In some instances, the structurally-stabilized peptide comprises a phenylalanine at the amino acid corresponding to F19 of SEQ ID NO: 102. In some instances, the structurally-stabilized peptide comprises a tryptophan at the amino acid corresponding to W23 of SEQ ID NO: 102.

In some instances, the structurally-stabilized peptide selectively binds to HDMX and comprises the amino acid sequence LTF8EYWAQBXSAA, wherein 8 is a first stapling amino acid, X is a second stapling amino acid, and B is cyclobutyl alanine (SEQ ID NO:55), with 1 to 10 (1, 2, 3, 4, 5, 6, 7, 8, 9, or 10) amino acid substitutions relative to the amino acid sequence of SEQ ID NO:55, wherein the 1 to 10 amino acid substitutions are not at positions 4 or 11 of SEQ ID NO:55, wherein at least one of the amino acid substitutions is at the amino acid position corresponding to E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid. In some instances, the amino acid substitution at the amino acid position corresponding to L26 of SEQ ID NO: 102 is with any amino acid other than phenylalanine, isoleucine, norleucine, and leucine (e.g., a charged amino acid (e.g., arginine or glutamic acid), alanine, a hydrophobic amino acid less hydrophobic than leucine, or a hydrophilic amino acid). In some instances, the structurally-stabilized peptide comprises a phenylalanine at the amino acid position corresponding F19 of SEQ ID NO: 102. In some instances, the structurally-stabilized peptide comprises a tryptophan at the amino acid position corresponding W23 of SEQ ID NO: 102.

In some instances, the structurally-stabilized peptide selectively binds to HDMX and comprises the amino acid sequence LSQETF8DLWKLLXEN, wherein 8 is a first stapling amino acid and X is a second stapling amino acid (SEQ ID NO: 1), with 1 to 10 (1, 2, 3, 4, 5, 6, 7, 8, 9, or 10) amino acid substitutions relative to the amino acid sequence of SEQ ID NO: 1, wherein the 1 to 10 amino acid substitutions are not at positions 7 or 14 of SEQ ID NO:1, wherein at least one of the amino acid substitutions is at the amino acid position corresponding to E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid. In some instances, the amino acid substitution at the amino acid position corresponding to L26 of SEQ ID NO: 102 is with any amino acid other than phenylalanine, isoleucine, norleucine, and leucine (e.g., a charged amino acid (e.g., arginine or glutamic acid), alanine, a hydrophobic amino acid less hydrophobic than leucine, or a hydrophilic amino acid). In some instances, the structurally-stabilized peptide comprises a phenylalanine at the amino acid position corresponding F19 of SEQ ID NO: 102. In some instances, the structurally-stabilized peptide comprises a tryptophan at the amino acid position corresponding W23 of SEQ ID NO: 102.

In some instances, the structurally-stabilized peptide selectively binds to HDMX and comprises the amino acid sequence QSQQTF8NLWRLLXQN, wherein 8 is a first stapling amino acid and X is a second stapling amino acid (SEQ ID NO:95), with 1 to 10 (1, 2, 3, 4, 5, 6, 7, 8, 9, or 10) amino acid substitutions relative to the amino acid sequence of SEQ ID NO:95, wherein the 1 to 10 amino acid substitutions are not at positions 7 or 14 of SEQ ID NO:95, wherein at least one of the amino acid substitutions is at the amino acid position corresponding to E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid. In some instances, the amino acid substitution at the amino acid position corresponding to L26 of SEQ ID NO: 102 is with any amino acid other than phenylalanine, isoleucine, norleucine, and leucine (e.g., a charged amino acid (e.g., arginine or glutamic acid), alanine, a hydrophobic amino acid less hydrophobic than leucine, or a hydrophilic amino acid). In some instances, the structurally-stabilized peptide comprises a phenylalanine at the amino acid position corresponding F19 of SEQ ID NO: 102. In some instances, the structurally-stabilized peptide comprises a tryptophan at the amino acid position corresponding W23 of SEQ ID NO: 102.

In some instances, the structurally-stabilized (e.g., stapled) peptide is a cross-linked version of a peptide comprising or consisting of the amino acid sequence of any one of SEQ ID NOs: 105-140. For example, two or more amino acids of these peptides are replaced by a stapling amino acid (e.g., an α-methyl, α-alkenyl non-natural amino acid). In some instances, the stapled peptide is a hydrocarbon stapled version of a peptide comprising or consisting of the amino acid sequence of any one of SEQ ID NOs: 105-140. In some instances, the stapled peptide is a peptide comprising or consisting of the amino acid sequence of any one SEQ ID NOs: 105-140, except that at least two (e.g., 2, 3, 4, 5, 6) amino acids of the amino acid sequence of any one of SEQ ID NOs: 105-140, respectively, are replaced with a non-natural amino acid capable of forming a staple or stitch (e.g., non-natural amino acids with olefinic side chains, e.g., (S)-2-(4′-pentenyl)Alanine, (R)-2-(4′-pentenyl)Alanine, (R)-2-(7′-octenyl)Alanine, and/or (S)-2-(7′-octenyl)Alanine). In some instances, the stapled peptide is a peptide comprising or consisting of the amino acid sequence of any one of SEQ ID NOs: 105-140 or comprising 1 to 13 (i.e., 1, 2, 3, 4, 5, 6, 7, 8, 9, 10, 11, 12, or 13) amino acid substitutions, deletions and/or insertions therein (in addition to the two or more amino acids replaced by stapling amino acids). In certain instances, the stapled peptide includes at least two (e.g., 2, 3, 4, 5, 6) amino acid substitutions, wherein the substituted amino acids are separated by two, three, or six amino acids, and wherein the substituted amino acids are non-natural amino acids with olefinic side chains (e.g., (S)-2-(4′-pentenyl)Alanine, (R)-2-(4′-pentenyl)Alanine, (R)-2-(7′-octenyl)Alanine, and/or (S)-2-(7′-octenyl)Alanine). There are many known non-natural amino acids that may be used as stapling amino acids or stitching amino acids, any of which may be included in the peptides of the present disclosure. α,α-disubstituted non-natural amino acids with olefinic side chains that can be cross-linked (optionally by ring closing metathesis reaction) are examples of non-natural amino acids that may be used as stapling amino acids. Another example of a non-natural amino acid that may be used as a stapling amino acid is an α-methyl, α-alkenyl non-natural amino acid. Some additional examples of non-natural amino acids that may be used as stapling amino acids or stitching amino acids are: (R)-2-(7′-octenyl)Alanine, (S)-2-(7′-octenyl)Alanine, (S)-2-(4′-pentenyl)Alanine, (R)-2-(4′-pentenyl)Alanine, (R)-2-(2′-propenyl)alanine 2,2-Bis(4′-pentenyl)glycine, and 2,2-Bis(7′-octenyl)glycine. Additionally, amino acids can be derivatized to include amino acid residues that are hydroxylated, phosphorylated, sulfonated, acylated, or glycosylated. When a stitch is present instead, or in addition to, a staple, a central bridging amino acid with two alkenyl residues is employed. When forming stitches, the central bridging non-natural amino acid can contain two alkenyl residues, such as in 2,2-Bis(4′-pentenyl)glycine and 2,2-Bis(7′-octenyl)glycine.

Hydrocarbon stapled peptides include one or more tethers (linkages) between two non-natural amino acids, which tether significantly enhances the α-helical secondary structure of the peptide. Generally, the tether extends across the length of one or two helical turns (i.e., about 3, 4 or about 7 amino acids). Accordingly, amino acids positioned at i and i+3; i and i+4; or i and i+7 are ideal candidates for chemical modification and cross-linking. Thus, for example, where a peptide has the sequence ... X1, X2, X3, X4, X5, X6, X7, X8, X9 ..., cross-links between X1 and X4, or between X1 and X5, or between X1 and X8 are useful hydrocarbon stapled forms of that peptide, as are cross-links between X2 and X5, or between X2 and X6, or between X2 and X9, etc. (i.e., forming an “i, i+3 staple”, an “i, i+4 staple”, or an “i, i+7 staple”, respectively). The use of multiple cross-links (e.g., 2, 3, 4, or more) is also contemplated. The use of multiple cross-links is very effective at stabilizing and optimizing the peptide, especially with increasing peptide length. Thus, the disclosure encompasses the incorporation of more than one cross-link within the peptide sequence to either further stabilize the sequence or facilitate the structural-stabilization, proteolytic resistance, acid stability, thermal stability, cellular permeability, and/or biological activity enhancement of longer peptide stretches. Additional description regarding making and use of hydrocarbon stapled peptides can be found, e.g., in U.S. Pat. Publication Nos. 2012/0172285, 2010/0286057, and 2005/0250680, the contents of all of which are incorporated by reference herein in their entireties.

In certain instances when a staple is at the i and i+3 residues, (R)-2-(2′-propenyl)alanine and (S)-2-(4′-pentenyl)Alanine are substituted for the amino acids at those positions, respectively. In certain instances when a staple is at the i and i+3 residues, (S)-2-(4′-pentenyl)Alanine and (R)-2-(2′-propenyl)alanine are substituted for the amino acids at those positions, respectively. In certain instances when a staple is at the i and i+3 residues, (R)-2-(4′-pentenyl)Alanine and (S)-2-(4′-pentenyl)Alanine are substituted for the amino acids at those positions, respectively. In certain instances when a staple is at the i and i+3 residues, (S)-2-(4′-pentenyl)Alanine and (R)-2-(4′-pentenyl)Alanine are substituted for the amino acids at those positions, respectively. In certain instances when a staple is at the i and i+4 residues, (S)-2-(4′-pentenyl)Alanine is substituted for the amino acids at each of i and i+4. In certain instances when a staple is at the i and i+7 residues, (S)-2-(4′-pentenyl)Alanine and (R)-2-(7′-octenyl)Alanine are substituted for the amino acids at i and i+7, respectively. In certain instances when a staple is at the i and i+7 residues, (R)-2-(7′-octenyl)Alanine and (S)-2-(4′-pentenyl)Alanine are substituted for the amino acids at i and i+7, respectively. In certain instances when a stitch is at the i, i+4, and i+11 residues, (S)-2-(4′-pentenyl)Alanine, 2,2-Bis(4′-pentenyl)glycine, and (S)-2-(7′-octenyl)Alanine are substituted for the amino acids at i, i+4, and i+11 positions, respectively. In certain instances when a stitch is at the i, i+7, and i+11 residues, (R)-2-(7′-octenyl)Alanine, 2,2-Bis(4′-pentenyl)glycine, and (S)-2-(4′-pentenyl)Alanine are substituted for the amino acids at i, i+7, and i+11 positions, respectively. In certain instances when a stitch is at the i, i+7, and i+14 residues, (R)-2-(7′-octenyl)Alanine, 2,2-Bis(4′-pentenyl)glycine, and (S)-2-(7′-octenyl)Alanine are substituted for the amino acids at i, i+7, and i+14, respectively. In certain instances when a stitch is at the i, i+7, and i+14 residues, (S)-2-(7′-octenyl)Alanine, 2,2-Bis(4′-pentenyl)glycine, and (R)-2-(7′-octenyl)Alanine are substituted for the amino acids at i, i+7, and i+14, respectively. In certain instances when a stitch is at the i, i+7, and i+14 residues, (R)-2-(4′-pentenyl)Alanine, 2,2-Bis(7′-octenyl)glycine, and (S)-2-(4′-pentenyl)Alanine are substituted for the amino acids at i, i+7, and i+14, respectively. In certain instances when a stitch is at the i, i+7, and i+14 residues, (S)-2-(4′-pentenyl)Alanine, 2,2-Bis(7′-octenyl)glycine, and (R)-2-(4′-pentenyl)Alanine are substituted for the amino acids at i, i+7, and i+14, respectively.

In a peptide to be stapled or stitched, amino acids that interfere with (e.g., inhibit or reduce the efficiency of) the stapling/stitching reaction should be substituted with amino acids that do not interfere with (e.g., do not inhibit or do not substantially reduce the efficiency of) the stapling/stitching reaction. For example, methionine (Met, M) may interfere with the stapling reaction; thus, in certain instances, the methionine(s) in a peptide to be stapled is replaced with, e.g., norleucine(s).

In some instances, the staple(s) is located at the amino acid positions in a p53 peptide (e.g., an HDMX-selective peptide) corresponding to positions 20 and 27 of human p53 (SEQ ID NO: 102) (i.e., positions 7 and 14 of SEQ ID NO:99, positions 7 and 14 of SEQ ID NO: 100, or positions 4 and 11 of SEQ ID NO: 101).

In some instances in which the p53 peptide comprises or consists of the amino acid sequence of any one of SEQ ID NOs: 105-140 or comprising 1 to 13 (i.e., 1, 2, 3, 4, 5, 6, 7, 8, 9, 10, 11, 12, or 13) amino acid substitutions, deletions and/or insertions therein (in addition to the two or more amino acids replaced by stapling amino acids), the staple(s) is located at the amino acid positions in a p53 peptide (e.g., an HDMX-selective peptide) corresponding to positions 20 and 27 of human p53 (SEQ ID NO: 102) (i.e., positions 7 and 14 of SEQ ID NO:99, positions 7 and 14 of SEQ ID NO: 100, or positions 4 and 11 of SEQ ID NO: 101).

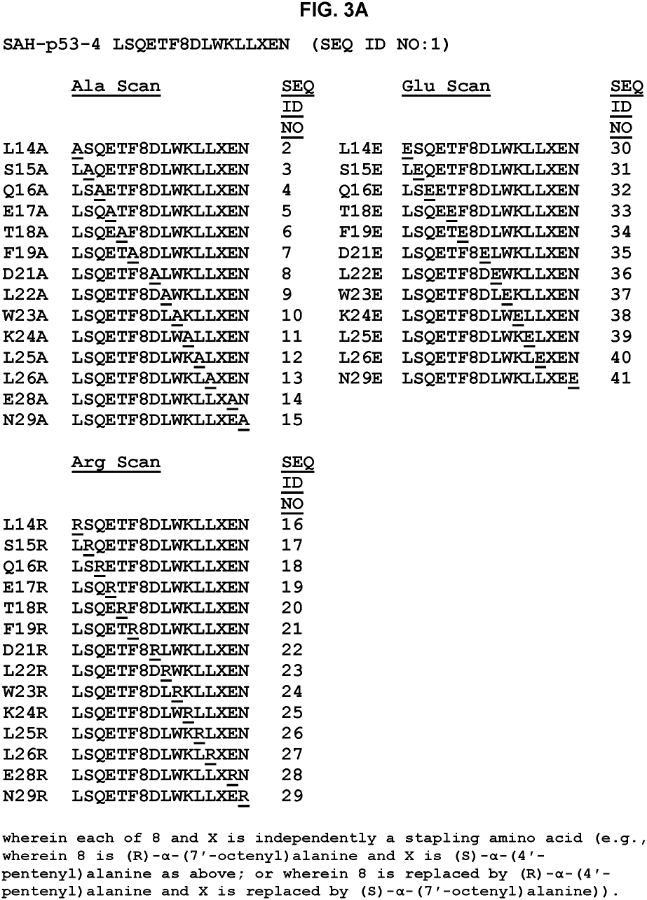

In some instances, the structurally-stabilized peptide comprises or consists of an amino acid sequence set forth in FIG. 3A (excluding SEQ ID NO: 1, e.g., the amino acid sequence of any one of SEQ ID NOs:2-29) with 0 to 8 (e.g., 0, 1, 2, 3, 4, 5, 6, 7, or 8) amino acid substitutions. In some instances, the amino acid sequence of any one of SEQ ID NOs: 5, 7, 8, 10, 13, 21, 23, 24, 27, 29, 32, 34, 37, 38, and 40 has selectivity to HDMX. In some instances, the amino acid sequence of any one of SEQ ID NOs: 3, 11, 17, 20, 30, and 31 has selectivity to HDM2. In some instances, the amino acid sequence of any one of SEQ ID NOs: 2, 4, 6, 9, 12, 14-16, 18, 19, 22, 25, 26, 28, 33, 35, 36, 39, and 41 has dual selectivity to HDMX and HDM2. In some instances, the structurally-stabilized peptide comprises or consists of a stapled form of a peptide described in FIG. 3A (excluding SEQ ID NO: 1, e.g., the amino acid sequence of any one of SEQ ID NOs:2-29), i.e., the stapled peptide is the product of one or more ring-closing metathesis reaction(s) on a peptide comprising or consisting of an amino acid sequence described in FIG. 3A (excluding SEQ ID NO:1, e.g., the amino acid sequence of any one of SEQ ID NOs:2-29).

In some instances, the structurally-stabilized peptide comprises or consists of an amino acid sequence set forth in FIG. 3B (e.g., the amino acid sequence of any one of SEQ ID NOs:42-54) with 0 to 8 (e.g., 0, 1, 2, 3, 4, 5, 6, 7, or 8) amino acid substitutions. In some instances, the amino acid sequence of any one of SEQ ID NOs: 42-46 and 48-51 has selectivity to HDMX. In some instances, the amino acid sequence of any one of SEQ ID NO:47 has selectivity to HDM2. In some instances, the structurally-stabilized peptide comprises or consists of a stapled form of a peptide described in FIG. 3B (e.g., the amino acid sequence of any one of SEQ ID NOs:42-54), i.e., the stapled peptide is the product of one or more ring-closing metathesis reaction(s) on a peptide comprising or consisting of an amino acid sequence described in FIG. 3B (e.g., the amino acid sequence of any one of SEQ ID NOs:42-54).

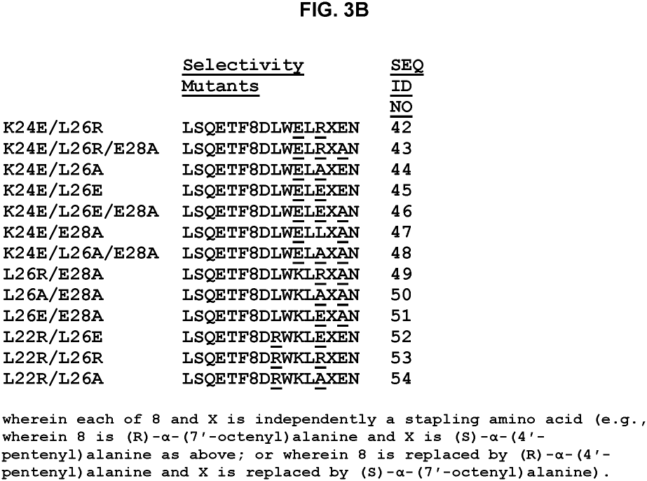

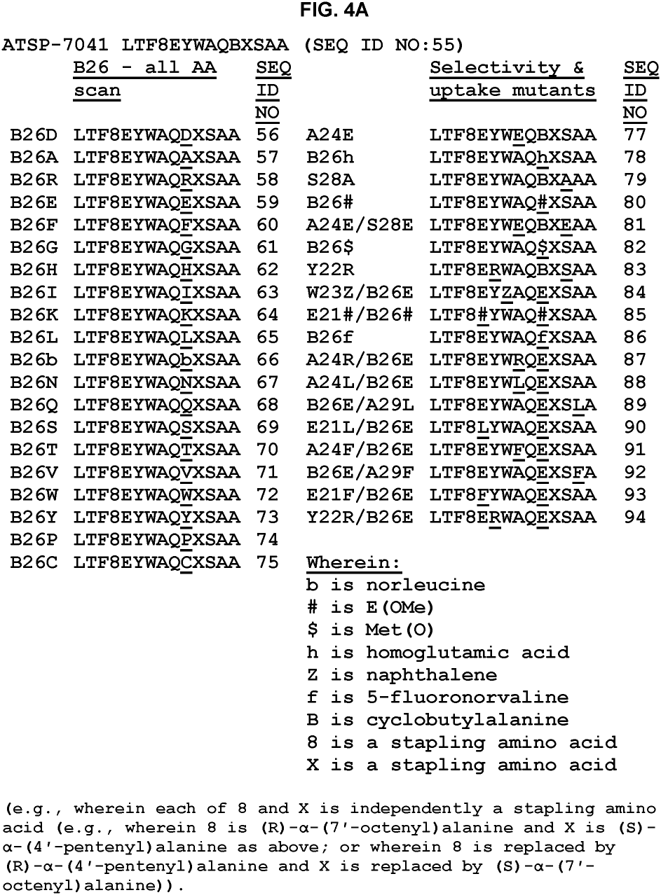

In some instances, the structurally-stabilized peptide comprises or consists of an amino acid sequence set forth in FIG. 4A (excluding SEQ ID NO:55, e.g., the amino acid sequence of any one of SEQ ID NOs:56-94) with 0 to 8 (e.g., 0, 1, 2, 3, 4, 5, 6, 7, or 8) amino acid substitutions. In some instances, the amino acid sequence of any one of SEQ ID NOs: 56-59, 61, 62, 64, 67-76, 78, 80, or 86-94 or or (SEQ ID NO:84)-napthalene-(SEQ ID NO:238) has selectivity to HDMX. In some instances, the amino acid sequence of any one of SEQ ID NOs: 60, 63, 65, 66, 77, 79, and 81 has dual-selectivity to HDMX and HDM2. In some instances, the structurally-stabilized peptide comprises or consists of a stapled form of a peptide described in FIG. 4A (excluding SEQ ID NO:55, e.g., the amino acid sequence of any one of SEQ ID NOs:56-94), i.e., the stapled peptide is the product of one or more ring-closing metathesis reaction(s) on a peptide comprising or consisting of an amino acid sequence described in FIG. 4A (excluding SEQ ID NO:55, e.g., the amino acid sequence of any one of SEQ ID NOs: 56-94).

In some instances, the structurally-stabilized peptide comprises or consists of an amino acid sequence set forth in FIG. 4B (excluding SEQ ID NO:95, e.g., the amino acid sequence of any one of SEQ ID NOs:96-98) with 0 to 8 (e.g., 0, 1, 2, 3, 4, 5, 6, 7, or 8) amino acid substitutions. In some instances, the amino acid sequence of any one of SEQ ID NOs: 96-98 has selectivity to HDMX. In some instances, the structurally-stabilized peptide comprises or consists of a stapled form of a peptide described in FIG. 4B (excluding SEQ ID NO:95, e.g., the amino acid sequence of any one of SEQ ID NOs:96-98), i.e., the stapled peptide is the product of one or more ring-closing metathesis reaction(s) on a peptide comprising or consisting of an amino acid sequence described in FIG. 4B (excluding SEQ ID NO:95, e.g., the amino acid sequence of any one of SEQ ID NOs:96-98).

In some instances, amino acids in the amino acid sequence of any one of SEQ ID NOs: 2-98 corresponding to amino acids in the interacting face of p53 (e.g., the interacting face of the transactivation domain of p53) can be conserved or can be conservative substitutions of the amino acids present in the interacting face of p53 (e.g., the interacting face of the transactivation domain of p53). In contrast, in some instances, amino acids outside the interacting face can have at least or about 30%, at least or about 40%, at least or about 50%, at least or about 60%, at least or about 70%, at least or about 80%, at least or about 90%, at least or about 95%, at least or about 98%, at least or about 99%, or 100% identity to those amino acids outside the interacting face of the peptide in the amino acid sequence of any one of SEQ ID NOs: 96-98. Alternatively or in addition, amino acids outside those in the interacting face can include amino acid substitutions and/or deletions, whether conservative or not. For example, amino acids outside those in the interacting face can include 1, 2, 3, 4, 5, 6, 7, 8, less than 10, less than 5, less than 4, less than 3, or less than 2 amino acid substitutions, deletions, and/or additions, whether conservative or not.

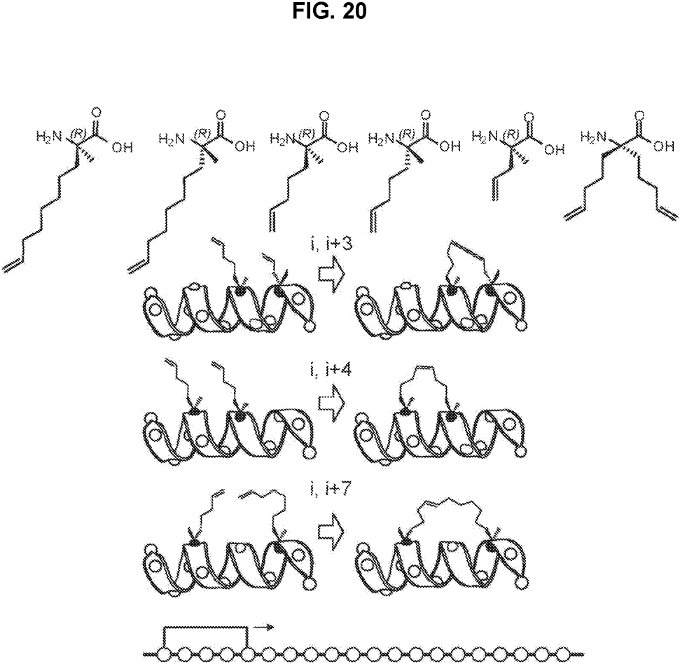

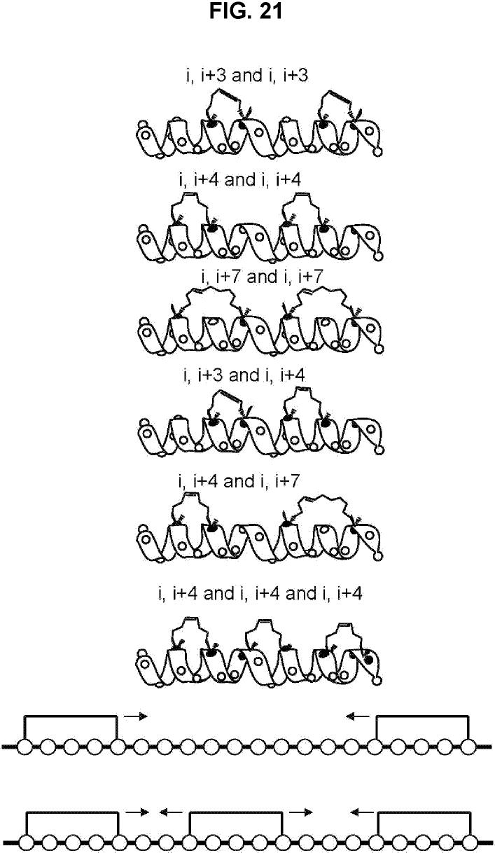

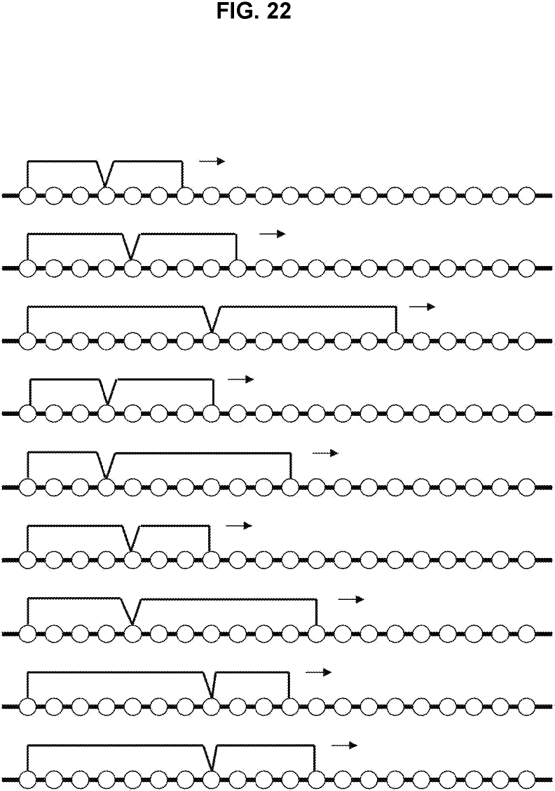

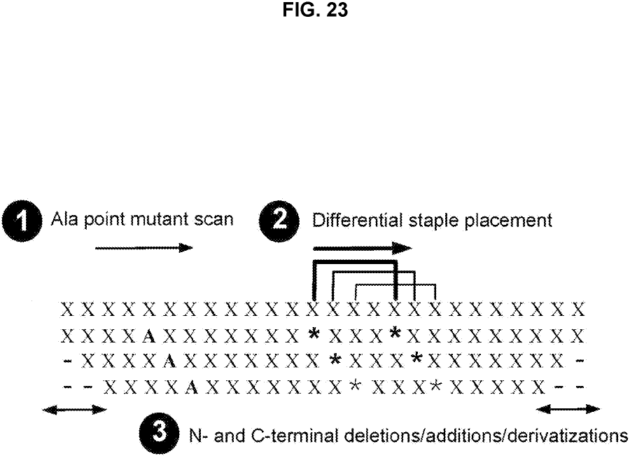

FIG. 20 top panel shows exemplary chemical structures of non-natural amino acids that can be used to generate various cross-linked compounds (i.e., “stapling amino acids” or “stitching amino acids”). FIG. 20 middle panel illustrates peptides with hydrocarbon cross-links between positions i and i+3; i and i+4; and i and i+7 residues. FIG. 20 bottom panel illustrates a staple walk along a peptide sequence. FIG. 21 shows various peptide sequences with double and triple stapling strategies, and exemplary staple walks. FIG. 22 illustrates exemplary staple walks using various lengths of branched stitched moieties. FIG. 23 illustrates peptide variants based on point mutant and staple scans, and N- and C-terminal deletions, additions, and/or derivatizations.

In one aspect, the structurally-stabilized peptide comprises Formula (I),

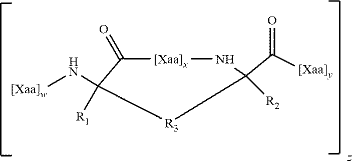

or a pharmaceutically acceptable salt thereof, wherein:
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄—K—R_{4]n}; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

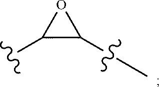

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);
and each Xaa is independently an amino acid, and wherein the structurally-stabilized peptide preferentially binds HDMX over HDM2. In some instances, the structurally-stabilized peptide also exhibits cancer cell (e.g., OCI-AML2) cytotoxicity.

In some instances, each of the [Xaa]_{w} of Formula (I), [Xaa]ₓ of Formula (I), and [Xaa]_{y} of Formula (I) is as described for any one of constructs 1-92 of Table 4. For example, for a stabilized peptide comprising the [Xaa]_{w}, the [Xaa]ₓ, and the [Xaa]_{y} of construct 1 of Table 4, [Xaa]_{w} is ASQETF (SEQ ID NO: 150), [Xaa]ₓ is DLWKLL (SEQ ID NO:151), and [Xaa]_{y} is EN. As another example, for a stabilized peptide comprising the [Xaa]_{w}, the [Xaa]ₓ, and the [Xaa]_{y} of construct 2 of Table 4, [Xaa]_{w} is LAQETF (SEQ ID NO: 152), [Xaa]ₓ is DLWKLL (SEQ ID NO: 151), and [Xaa]_{y} is EN.

TABLE 4

[Xaa]_{w}, [Xaa]ₓ, and [Xaa]_{y} sequences for Formula (I) constructs 1-92

| Construct | [Xaa]_{w} | [Xaa]ₓ | [Xaa]_{y} |
|---|---|---|---|
| 1 | ASQETF (SEQ ID NO:150) | DLWKLL (SEQ ID NO:151) | EN |
| 2 | LAQETF (SEQ ID NO:152) | DLWKLL (SEQ ID NO:151) | EN |
| 3 | LSAETF (SEQ ID NO:153) | DLWKLL (SEQ ID NO:151) | EN |
| 4 | LSQATF (SEQ ID NO:154 | DLWKLL (SEQ ID NO:151) | EN |
| 5 | LSQEAF (SEQ ID NO:155) | DLWKLL (SEQ ID NO:151) | EN |
| 6 | LSQETA (SEQ ID NO:156) | DLWKLL (SEQ ID NO:151) | EN |
| 7 | LSQETF (SEQ ID NO:157) | ALWKLL (SEQ ID NO:158) | EN |
| 8 | LSQETF (SEQ ID NO:157) | DAWKLL (SEQ ID NO:159) | EN |
| 9 | LSQETF (SEQ ID NO:157) | DLAKLL (SEQ ID NO:160) | EN |
| 10 | LSQETF (SEQ ID NO:157) | DLWALL (SEQ ID NO:161) | EN |
| 11 | LSQETF (SEQ ID NO:157) | DLWKAL (SEQ ID NO:234) | EN |
| 12 | LSQETF (SEQ ID NO:157) | DLWKLA (SEQ ID NO:235) | EN |
| 13 | LSQETF (SEQ ID NO:157) | DLWKLL (SEQ ID NO:151) | AN |
| 14 | LSQETF (SEQ ID NO:157) | DLWKLL (SEQ ID NO:151) | EA |
| 15 | RSQETF(SEQ ID NO:162) | DLWKLL (SEQ ID NO:151) | EN |
| 16 | LRQETF (SEQ ID NO:163) | DLWKLL (SEQ ID NO:151) | EN |
| 17 | LSRETF (SEQ ID NO:164) | DLWKLL (SEQ ID NO:151) | EN |
| 18 | LSQRTF (SEQ ID NO:165) | DLWKLL (SEQ ID NO:151) | EN |
| 19 | LSQERF (SEQ ID NO:166) | DLWKLL (SEQ ID NO:151) | EN |
| 20 | LSQETR (SEQ ID NO:167) | DLWKLL (SEQ ID NO:151) | EN |
| 21 | LSQETF (SEQ ID NO:157) | RLWKLL (SEQ ID NO:168) | EN |
| 22 | LSQETF (SEQ ID NO:157) | DRWKLL (SEQ ID NO:169) | EN |
| 23 | LSQETF (SEQ ID NO:157) | DLRKLL (SEQ ID NO:170) | EN |
| 24 | LSQETF (SEQ ID NO:157) | DLWRLL (SEQ ID NO:171) | EN |
| 25 | LSQETF (SEQ ID NO:157) | DLWKRL (SEQ ID NO:172) | EN |
| 26 | LSQETF (SEQ ID NO:157) | DLWKLR (SEQ ID NO:173) | EN |
| 27 | LSQETF (SEQ ID NO:157) | DLWKLL (SEQ ID NO:151) | RN |
| 28 | LSQETF (SEQ ID NO:157) | DLWKLL (SEQ ID NO:151) | ER |
| 29 | ESQETF (SEQ ID NO:174) | DLWKLL (SEQ ID NO:151) | EN |
| 30 | LEQETF (SEQ ID NO:175) | DLWKLL (SEQ ID NO:151) | EN |
| 31 | LSEETF (SEQ ID NO:176) | DLWKLL (SEQ ID NO:151) | EN |
| 32 | LSQEEF (SEQ ID NO:177) | DLWKLL (SEQ ID NO:151) | EN |
| 33 | LSQETE (SEQ ID NO:178) | DLWKLL (SEQ ID NO:151) | EN |
| 34 | LSQETF (SEQ ID NO:157) | ELWKLL (SEQ ID NO:179) | EN |
| 35 | LSQETF (SEQ ID NO:157) | DEWKLL (SEQ ID NO:180) | EN |
| 36 | LSQETF (SEQ ID NO:157) | DLEKLL (SEQ ID NO:181) | EN |
| 37 | LSQETF (SEQ ID NO:157) | DLWELL (SEQ ID NO:182) | EN |
| 38 | LSQETF (SEQ ID NO:157) | DLWKEL (SEQ ID NO:183) | EN |
| 39 | LSQETF (SEQ ID NO:157) | DLWKLE (SEQ ID NO:184) | EN |
| 40 | LSQETF (SEQ ID NO:157) | DLWKLL (SEQ ID NO:151) | EE |
| 41 | LSQETF (SEQ ID NO:157) | DLWELR (SEQ ID NO:185) | EN |
| 42 | LSQETF (SEQ ID NO:157) | DLWELR (SEQ ID NO:185) | AN |
| 43 | LSQETF (SEQ ID NO:157) | DLWELA (SEQ ID NO:186) | EN |
| 44 | LSQETF (SEQ ID NO:157) | DLWELE (SEQ ID NO:187) | EN |
| 45 | LSQETF (SEQ ID NO:157) | DLWELE (SEQ ID NO:187) | AN |
| 46 | LSQETF (SEQ ID NO:157) | DLWELL (SEQ ID NO:182) | AN |
| 47 | LSQETF (SEQ ID NO:157) | DLWELA (SEQ ID NO:186) | AN |
| 48 | LSQETF (SEQ ID NO:157) | DLWKLR (SEQ ID NO:173) | AN |
| 49 | LSQETF (SEQ ID NO:157) | DLWKLA (SEQ ID NO:235) | AN |
| 50 | LSQETF (SEQ ID NO:157) | DLWKLE (SEQ ID NO:184) | AN |
| 51 | LSQETF (SEQ ID NO:157) | DRWKLE (SEQ ID NO:188) | EN |
| 52 | LSQETF (SEQ ID NO:157) | DRWKLR (SEQ ID NO:189) | EN |
| 53 | LSQETF (SEQ ID NO:157) | DRWKLA (SEQ ID NO:190) | EN |
| 54 | LTF | EYWAQD (SEQ ID NO:191) | SAA |
| 55 | LTF | EYWAQA (SEQ ID NO:192) | SAA |
| 56 | LTF | EYWAQR (SEQ ID NO:193) | SAA |
| 57 | LTF | EYWAQE (SEQ ID NO:194) | SAA |
| 58 | LTF | EYWAQF (SEQ ID NO:195) | SAA |
| 59 | LTF | EYWAQG (SEQ ID NO:196) | SAA |
| 60 | LTF | EYWAQH (SEQ ID NO:197) | SAA |
| 61 | LTF | EYWAQI (SEQ ID NO:198) | SAA |
| 62 | LTF | EYWAQK (SEQ ID NO:199) | SAA |
| 63 | LTF | EYWAQL (SEQ ID NO:200) | SAA |
| 64 | LTF | EYWAQb (SEQ ID NO:201) | SAA |
| 65 | LTF | EYWAQN (SEQ ID NO:202) | SAA |
| 66 | LTF | EYWAQQ (SEQ ID NO:203) | SAA |
| 67 | LTF | EYWAQS (SEQ ID NO:204) | SAA |
| 68 | LTF | EYWAQT (SEQ ID NO:205) | SAA |
| 69 | LTF | EYWAQV (SEQ ID NO:206) | SAA |
| 70 | LTF | EYWAQW (SEQ ID NO:207) | SAA |
| 71 | LTF | EYWAQY (SEQ ID NO:208) | SAA |
| 72 | LTF | EYWAQP (SEQ ID NO:209) | SAA |
| 73 | LTF | EYWAQC (SEQ ID NO:210) | SAA |
| 74 | LTF | ERWAQB (SEQ ID NO:211) | SAA |
| 75 | LTF | EYWEQB (SEQ ID NO:212) | (S/E)AA |
| 76 | LTF | EYWAQh (SEQ ID NO:213) | SAA |
| 77 | LTF | EYWAQB (SEQ ID NO:214) | AAA |
| 78 | LTF | EYWAQ# (SEQ ID NO:228) | SAA |
| 79 | LTF | EYWEQB (SEQ ID NO:215) | AAA |
| 80 | LTF | EYWAQ$ (SEQ ID NO:227) | SAA |
| 81 | LTF | ERWAQB (SEQ ID NO:216) | SAA |
| 82 | LTF | EYZAQE (SEQ ID NO:217) | SAA |
| 83 | LTF | EYWAQf (SEQ ID NO:218) | SAA |
| 84 | LTF | EYWRQE (SEQ ID NO:219) | SAA |
| 85 | LTF | EYWLQE (SEQ ID NO:220) | SAA |
| 86 | LTF | EYWAQE (SEQ ID NO:194) | SLA |
| 87 | LTF | LYWAQE (SEQ ID NO:221) | SAA |
| 88 | LTF | EYWFQE (SEQ ID NO:222) | SAA |
| 89 | LTF | EYWAQE (SEQ ID NO:194) | SFA |
| 90 | LTF | FYWAQE (SEQ ID NO:223) | SAA |
| 91 | LTF | ERWAQE (SEQ ID NO:224) | SAA |
| 92 | QSQQTF (SEQ ID NO:225) | NLWRLE (SEQ ID NO:226) | QN |

In certain instances, the sequences set forth above in Table 4 can have at least one (e.g., 1, 2, 3, 4, 5, 6) amino acid substitution or deletion. The p53 peptides can include any amino acid sequence described herein.

The tether of Formula (I) can include an alkyl, alkenyl, or alkynyl moiety (e.g., Cs, Cs, C₁₁, or C₁₂ alkyl, a C₅, Cs, or C₁₁ alkenyl, or C₅, Cs, C₁₁, or C₁₂ alkynyl). The tethered amino acid can be alpha disubstituted (e.g., C₁-C₃ or methyl).

In some instances of Formula (I), x is 2, 3, or 6. In some instances of Formula (I), each y is independently an integer between 0 and 15, or 3 and 15. In some instances of Formula (I), R₁ and R₂ are each independently H or C₁-C₆ alkyl. In some instances of Formula (I), R₁ and R₂ are each independently C₁-C₃ alkyl. In some instances or Formula (I), at least one of R₁ and R₂ are methyl. For example, R₁ and R₂ can both be methyl. In some instances of Formula (I), R₃ is alkyl (e.g., Cs alkyl) and x is 3. In some instances of Formula (I), R₃ is C₁₁ alkyl and x is 6. In some instances of Formula (I), R₃ is alkenyl (e.g., Cs alkenyl) and x is 3. In some instances of Formula (I), x is 6 and R₃ is C₁₁ alkenyl. In some instances of Formula (I), R₃ is a straight chain alkyl, alkenyl, or alkynyl. In some instances of Formula (I), R₃ is —CH₂—CH₂—CH₂—CH═CH—CH₂—CH₂—CH₂—. In some instances of Formula (I) having an i, i+4 staple, R₃ is —CH₂—CH₂—CH₂—CH═CH—CH₂—CH₂—CH₂—. In some instances of Formula (I) having an i, i+7 staple, R₃ is —(CH₂)₆—CH═CH—(CH₂)₆—.

In another aspect of Formula (I), the two alpha, alpha disubstituted stereocenters are both in the R configuration or S configuration (e.g., i, i+4 cross-link), or one stereocenter is R and the other is S (e.g., i, i+4 cross-link). Thus, where Formula (I) is depicted as:

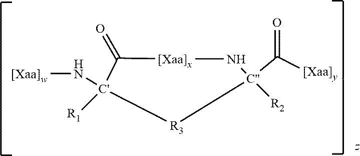

the C′ and C″ disubstituted stereocenters can both be in the R configuration or they can both be in the S configuration, e.g., when x is 3. When x is 6 in Formula (I), the C′ disubstituted stereocenter is in the R configuration and the C″ disubstituted stereocenter is in the S configuration. The R₃ double bond of Formula (I) can be in the E or Z stereochemical configuration.

In some instances of Formula (I), R₃ is [R₄—K—R₄]ₙ; and R₄ is a straight chain alkyl, alkenyl, or alkynyl.

As used herein, the term “Cᵢ₋ⱼ,” where i and j are integers, employed in combination with a chemical group, designates a range of the number of carbon atoms in the chemical group with i-j defining the range. For example, C₁-₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some instances, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some instances, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some instances, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some instances, the alkynyl moiety contains 2 to 6 or 2 to 4 carbon atoms.

As used herein, the term “cycloalkylalkyl,” employed alone or in combination with other terms, refers to a group of formula cycloalkyl-alkyl-. In some instances, the alkyl portion has 1 to 4, 1 to 3, 1 to 2, or 1 carbon atom(s). In some instances, the alkyl portion is methylene. In some instances, the cycloalkyl portion has 3 to 10 ring members or 3 to 7 ring members. In some instances, the cycloalkyl group is monocyclic or bicyclic. In some instances, the cycloalkyl portion is monocyclic. In some instances, the cycloalkyl portion is a C₃₋₇ monocyclic cycloalkyl group.

As used herein, the term “heteroarylalkyl,” employed alone or in combination with other terms, refers to a group of formula heteroaryl-alkyl-. In some instances, the alkyl portion has 1 to 4, 1 to 3, 1 to 2, or 1 carbon atom(s). In some instances, the alkyl portion is methylene. In some instances, the heteroaryl portion is a monocyclic or bicyclic group having 1, 2, 3, or 4 heteroatoms independently selected from nitrogen, sulfur and oxygen. In some instances, the heteroaryl portion has 5 to 10 carbon atoms.

As used herein, the term “substituted” means that a hydrogen atom is replaced by a non-hydrogen group. It is to be understood that substitution at a given atom is limited by valency.

As used herein, “halo” or “halogen”, employed alone or in combination with other terms, includes fluoro, chloro, bromo, and iodo. In some instances, halo is F or Cl.

In some instances, the disclosure features structurally-stabilized (e.g., stapled) peptides comprising the amino acid sequence of any one of SEQ ID NOs:105-140 or comprising 1 to 13 (i.e., 1, 2, 3, 4, 5, 6, 7, 8, 9, 10, 11, 12, or 13) amino acid substitutions, deletions and/or insertions therein (in addition to the two or more amino acids replaced by stapling amino acids), wherein: the side chains of two amino acids separated by two, three, or six amino acids are replaced by an internal staple, the side chains of three amino acids are replaced by an internal stitch, the side chains of four amino acids are replaced by two internal staples, or the side chains of five amino acids are replaced by the combination of an internal staple and an internal stitch. In some instances, the disclosure features structurally-stabilized (e.g., stapled) peptides comprising the amino acid sequence of any one of SEQ ID NOs: 105-140 or comprising 1 to 13 (i.e., 1, 2, 3, 4, 5, 6, 7, 8, 9, 10, 11, 12, or 13) amino acid substitutions, deletions and/or insertions therein (in addition to the two or more amino acids replaced by stapling amino acids), wherein the side chains of two amino acids separated by two, three, or six amino acids are replaced by an internal staple. In some instances, the disclosure features structurally-stabilized (e.g., stapled) peptides comprising the amino acid sequence of any one of SEQ ID NOs: 105-140 or comprising 1 to 13 (i.e., 1, 2, 3, 4, 5, 6, 7, 8, 9, 10, 11, 12, or 13) amino acid substitutions, deletions and/or insertions therein (in addition to the two or more amino acids replaced by stapling amino acids), wherein the side chains of two amino acids separated by three amino acids are replaced by an internal staple. In some instances, the disclosure features structurally-stabilized (e.g., stapled) peptides comprising the amino acid sequence of any one of SEQ ID NOs: 105-140 or comprising 1 to 13 (i.e., 1, 2, 3, 4, 5, 6, 7, 8, 9, 10, 11, 12, or 13) amino acid substitutions, deletions and/or insertions therein (in addition to the two or more amino acids replaced by stapling amino acids), wherein the side chains of two amino acids separated by six amino acids are replaced by an internal staple. The stapled or stitched peptide can be 5 or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids in length. In a specific instance, the stapled or stitched peptide is 5-30 (i.e., 5, 6, 7, 8, 9, 10, 11, 12, 13, 14, 15, 16, 17, 18, 19, 20, 21, 22, 23, 24, 25, 26, 27, 28, 29, 30) in length. In a specific instance, the stapled or stitched peptide is 10-30 (i.e., 10, 11, 12, 13, 14, 15, 16, 17, 18, 19, 20, 21, 22, 23, 24, 25, 26, 27, 28, 29, or 30) amino acids in length. In a specific instance, the stapled or stitched peptide is 15-30 (i.e., 15, 16, 17, 18, 19, 20, 21, 22, 23, 24, 25, 26, 27, 28, 29, or 30) amino acids in length. In a specific instance, the stapled or stitched peptide is 5-23 (i.e., 5, 6, 7, 8, 9, 10, 11, 12, 13, 14, 15, 16, 17, 18, 19, 20, 21, 22, or 23) in length. In a specific instance, the stapled or stitched peptide is 10-23 (i.e., 10, 11, 12, 13, 14, 15, 16, 17, 18, 19, 20, 21, 22, or 23) amino acids in length. In a specific instance, the stapled or stitched peptide is 15-23 (i.e., 15, 16, 17, 18, 19, 20, 21, 22, or 23) amino acids in length. In a specific instance, the stapled or stitched peptide is 23-30 (i.e., 23, 24, 25, 26, 27, 28, 29, or 30) amino acids in length. In a specific instance, the stapled or stitched peptide is 14 amino acids in length. In a specific instance, the stapled or stitched peptide is 16 amino acids in length. Exemplary stapled peptides are shown in FIG. 3A, FIG. 3B, FIG. 4A, and FIG. 4B, and are described in the Formula (I) constructs of Table 2. In one instance, the stapled peptide comprises or consists of a stapled version of the amino acid sequence of any one of SEQ ID NOs: 2-54, 56-94, or 96-98, (e.g., the product of a ring-closing metathesis reaction(s) performed on a peptide comprising the amino acid sequence of any one of SEQ ID NOs: 2-54, 56-94, or 96-98, respectively). In one instance, the stapled peptide comprises or consists of a stapled version of the amino acid sequence of any one of SEQ ID NOs: 2-54, 56-94, or 96-98, (e.g., the product of a ring-closing metathesis reaction(s) performed on a peptide comprising the amino acid sequence of any one of SEQ ID NOs: 2-54, 56-94 or 96-98, respectively).

Exemplary stapled peptides are shown in FIG. 3A, FIG. 3B, FIG. 4A, and FIG. 4B. In some instances, the stapled peptide comprises or consists of the amino acid sequence of SEQ ID NO: 2-54, 56-94, or 96-98.

In certain instances, the stapled peptide comprises or consists of a variant of the amino acid sequence set forth in any one of SEQ ID NOs:105-140, wherein two amino acids each separated by 6 amino acids (i.e., positions i and i+7) are modified to structurally stabilize the peptide (e.g., by substituting them with non-natural amino acids to permit hydrocarbon stitching, i.e., stapling amino acids).

In some instances, a structurally-stabilized (e.g., stapled) peptide described herein is selective for HDMX (e.g., SEQ ID NOs: 5, 7, 8, 10, 13, 21, 23, 24, 27, 29, 32, 34, 37, 38, 40, 42-46, 48-51, 56-59, 61, 62, 64, 67-76, 78, 80, or 86-94 or (SEQ ID NO:84)-napthalene-(SEQ ID NO:238)). In some instances, a structurally-stabilized peptide is selective for HDMX if a dual HDMX-HDM2 inhibitor and a selective HDMX inhibitor are equally active in a cancer cell of a subject. In some instances, a structurally-stabilized peptide is selective for HDMX if it has a binding affinity for HDMX that is at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher than the structurally-stabilized peptide’s binding affinity for HDM2. In some instances, binding affinity is determined using isothermal calorimetry or fluorescence polarization analyses.

In some instances, a structurally-stabilized (e.g., stapled) peptide described herein is selective for HDM2 (e.g., SEQ ID NOs: 3, 11, 17, 20, 30, 31, or 47). In some instances, a structurally-stabilized peptide is selective for HDM2 if the peptide has the same, similar, or greater binding affinity for HDM2-specific compared either to other HDM2 peptides or HDMX-HDM2 dual peptides. In some instances, a structurally-stabilized peptide is selective for HDM2 if it has a binding affinity for HDM2 that is at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher than the structurally-stabilized peptide’s binding affinity for HDMX. In some instances, binding affinity is determined using isothermal calorimetry or fluorescence polarization analyses.

In some instances, a structurally-stabilized (e.g., stapled) peptide described herein is selective for both HDMX and HDM2 (e.g., SEQ ID NOs: 2, 4, 6, 9, 12, 14-16, 18, 19, 22, 25, 26, 28, 33, 35, 36, 39, 41, 60, 63, 65, 66, 77, 79, and 81). In some instances, a structurally-stabilized peptide is selective for both HDMX and HDM2 if the peptide has the same, similar, or greater binding affinity for HDM2 and HDMX compared to other HDM2-specific peptides, to HDMX-specific peptides, or to other HDMX-HDM2 dual peptides. In some instances, a structurally-stabilized peptide is selective for both HDMX and HDM2 if it has a binding affinity for HDM2 that is less than 0.1-fold, less than 0.2-fold, less than 0.3-fold, less than 0.4-fold, less than 0.5-fold, less than 0.6-fold, less than 0.7-fold, less than 0.8-fold, less than 1-fold, less than 1.25-fold, or less than 1.5-fold higher or lower than the structurally-stabilized peptide’s binding affinity compared to other HDM2-specific peptides, to HDMX-specific peptides, or to other HDMX-HDM2 dual peptides. In some instances, binding affinity is determined using isothermal calorimetry or fluorescence polarization analyses.

While hydrocarbon tethers are common, other tethers can also be employed in the structurally-stabilized peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed with the p53 peptides described herein (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U. S. A., 97:3803-3808 (2000); Double-click stapling: Lau et al., Chem. Sci., 5: 1804-1809 (2014); Bis-lactam stapling: J. C. Phelan et al.,, J. Am. Chem. Soc., 119:455-460 (1997); and Bis-arylation stapling: A. M. Spokoyny et al., J. Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to i+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

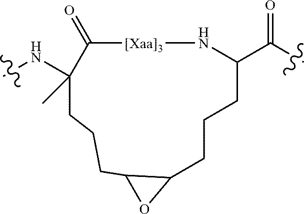

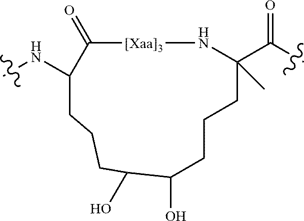

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the peptide or via the amino acid side chain. Other agents can be attached to the functionalized tether, e.g., an agent that facilitates entry of the peptide into cells.

In some instances, α-methyl, α-alkenyl non-natural amino acids are used in the peptide to improve the stability of the alpha helical secondary structure. However, α-methyl, α-alkenyl non-natural amino acids are not required, and instances using mono-alpha substituents (e.g., in the tethered amino acids) are also envisioned.

The structurally-stabilized (e.g., stapled) peptides can include a drug, a toxin, a derivative of polyethylene glycol; a second peptide; a carbohydrate, etc. Where a polymer or other agent is linked to the structurally-stabilized (e.g., stapled) peptide, it can be desirable for the composition to be substantially homogeneous.

The addition of polyethelene glycol (PEG) molecules can improve the pharmacokinetic and pharmacodynamic properties of the peptide. For example, PEGylation can reduce renal clearance and can result in a more stable plasma concentration. PEG is a water soluble polymer and can be represented as linked to the peptide as formula:

XO—(CH₂CH₂O)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the peptide. Y may also be a maleimide linkage to a thiol group (including but not limited to, the thiol group of cysteine). Other methods for linking PEG to a peptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. 6,348,558.

In certain instances, macromolecular polymer (e.g., PEG) is attached to a structurally-stabilized (e.g., stapled) peptide described herein through an intermediate linker. In certain instances, the linker is made up of from 1 to 20 amino acids linked by peptide bonds, wherein the amino acids are selected from the 20 naturally occurring amino acids. Some of these amino acids may be glycosylated, as is well understood by those in the art. In other instances, the 1 to 20 amino acids are selected from glycine, alanine, proline, asparagine, glutamine, and lysine. In other instances, a linker is made up of a majority of amino acids that are sterically unhindered, such as glycine and alanine. Non-peptide linkers are also possible. For example, alkyl linkers such as —NH(CH₂)ₙC(O)—, wherein n = 2-20 can be used. These alkyl linkers may further be substituted by any non-sterically hindering group such as lower alkyl (e.g., C₁-C₆) lower acyl, halogen (e.g., Cl, Br), CN, NH2, phenyl, etc. U.S. Pat. No. 5,446,090 describes a bifunctional PEG linker and its use in forming conjugates having a peptide at each of the PEG linker termini.

The structurally-stabilized (e.g., stapled) peptides can also be modified, e.g., to further facilitate cellular uptake or increase in vivo stability, in some instances. For example, acylating or PEGylating a structurally-stabilized peptide facilitates cellular uptake, increases bioavailability, increases blood circulation, alters pharmacokinetics, decreases immunogenicity and/or decreases the needed frequency of administration.

In some instances, the structurally-stabilized (e.g., stapled) peptides disclosed herein have an enhanced ability to penetrate cell membranes (e.g., relative to non-stabilized peptides). See, e.g., International Publication No. WO 2017/147283, which is incorporated by reference herein in its entirety. For instance, one or more negatively charged residues may be esterified to improve cell penetrance.

Methods of synthesizing the structurally-stabilized (e.g., stapled) peptides described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T.W. Greene and P.G.M. Wuts, Protective Groups in Organic Synthesis, 3d. Ed., John Wiley and Sons (1999); L. Fieser and M. Fieser, Fieser and Fieser’s Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this disclosure can be made by chemical synthesis methods, which are well known to the ordinarily skilled artisan. See, for example, Fields et al., Chapter 3 in Synthetic Peptides: A User’s Guide, ed. Grant, W. H. Freeman & Co., New York, N.Y., 1992, p. 77. Hence, peptides can be synthesized using the automated Merrifield techniques of solid phase synthesis with the α—NH₂ protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this disclosure, the amino acid sequence is reverse translated to obtain a nucleic acid sequence encoding the amino acid sequence. In some instances, the amino acid sequence is reverse translated to obtain a nucleic acid sequence encoding the amino acid sequence with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech. Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)-CHR) or CHR-C(O) wherein R is H or CH₃; and a fluoro-ketomethylene bond (C(O)-CFR or CFR-C(O) wherein R is H or F or CH₃.

The peptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof. α-methyl, α-alkenyl non-natural amino acids containing olefinic side chains of varying length can be synthesized by known methods (Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al., Current Protocols in Chemical Biology, 2011). For peptides where an i linked to i+4 staple is used (two turns of the helix stabilized): two (S)-2-(4′-pentenyl)Alanine amino acids may be used. Inhibitors are synthesized on a solid support using solid-phase peptide synthesis (SPPS) on MBHA resin (see, e.g., WO 2010/148335).

Fmoc-protected α-amino acids (other than the olefinic amino acids N-Fmoc-α,α-Bis(4′-pentenyl)glycine, (S)-N-Fmoc-α-(4′-pentenyl)alanine, (R)-N-Fmoc-α-(7′-octenyl)alanine, (R)-N-Fmoc-α-(7′-octenyl)alanine, and (R)-N-Fmoc-α-(4′-pentenyl)alanine), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, CA). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stapling or stitching, and purifying the peptides disclosed herein are also known in the art (see, e.g., Bird et. al., Methods in Enzymol., 446:369-386 (2008); Bird et al., Current Protocols in Chemical Biology, 2011; Walensky et al., Science, 305:1466-1470 (2004); Schafmeister et al., J. Am. Chem. Soc., 122:5891-5892 (2000); U.S. Pat. Application No. 12/525,123, filed Mar. 18, 2010; and U.S. Pat. No. 7,723,468, issued May 25, 2010, each of which are hereby incorporated by reference in their entirety).

In some instances, the structurally-stabilized (e.g., stapled) peptides are substantially free of non-structurally-stabilized peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific instance, a 50%/50% DMSO/NMP solution is used. The solution may be incubated for a period of 1, 6, 12 or 24 hours, following which the resin may be washed, for example with dichloromethane or NMP. In one instance, the resin is washed with NMP. Shaking and bubbling an inert gas into the solution may be performed.

Also provided herein is a method of producing a structurally-stabilized (e.g., stapled) peptide comprising: (a) stapling or stitching a p53 peptide (or variant thereof); and (b) isolating the stapled or stitched peptide.

Properties of the stabilized (e.g., stapled) peptides of the disclosure can be assayed, for example, using the methods described below and in the Examples.

Assays to Determine α-Helicity: Compounds are dissolved in an aqueous solution (e.g., 5 mM potassium phosphate solution at pH 7, or distilled H₂O, to concentrations of 25-50 µM). Circular dichroism (CD) spectra are obtained on a spectropolarimeter (e.g., Jasco J-710, Aviv) using standard measurement parameters (e.g., temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol. 130:208 (1986)).

Assays to Determine Melting Temperature (Tm): Cross-linked or the unmodified template peptides are dissolved in distilled H₂O or other buffer or solvent (e.g., at a final concentration of 50 µM) and Tm is determined by measuring the change in ellipticity over a temperature range (e.g., 4 to 95° C.) on a spectropolarimeter (e.g., Jasco J-710, Aviv) using standard parameters (e.g., wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In vitro Protease Resistance Assays: The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The stabilized peptides of the present disclosure may be subjected to in vitro enzymatic proteolysis (e.g., trypsin, chymotrypsin, pepsin) to assess for any change in degradation rate compared to a corresponding unstabilized or alternatively stapled or stitched peptide. For example, the stabilized peptide and a corresponding unstabilized peptide are incubated with trypsin agarose and the reactions quenched at various time points by centrifugation and subsequent HPLC injection to quantitate the residual substrate by ultraviolet absorption at 280 nm. Briefly, the stabilized peptide and its precursor (5 mcg) are incubated with trypsin agarose (Pierce) (S/E ~125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Stabilized peptides and/or a corresponding unstabilized peptide can be each incubated with fresh mouse, rat and/or human serum (e.g., 1-2 mL) at 37° C. for, e.g., 0, 1, 2, 4, 8, and 24 hours. Samples of differing stabilized peptide concentration may be prepared by serial dilution with serum. To determine the level of intact compound, the following procedure may be used: The samples are extracted, for example, by transferring 100 µL of sera to 2 ml centrifuge tubes followed by the addition of 10 µL of 50% formic acid and 500 µL acetonitrile and centrifugation at 14,000 RPM for 10 min at 4+/-2° C. The supernatants are then transferred to fresh 2 ml tubes and evaporated on Turbovap under N₂<10 psi, 37° C. The samples are reconstituted in 100 µL of 50:50 acetonitrile:water and submitted to LC-MS/MS analysis. Equivalent or similar procedures for testing ex vivo stability are known and may be used to determine stability of stabilized peptides in serum.

In vivo Protease Resistance Assays: A key benefit of peptide stapling or stitching is the translation of in vitro protease resistance into markedly improved pharmacokinetics in vivo. Protease resistance assays are known in the art and can be utilized to evaluate protease resistance of a structurally-stabilzied peptide described herein.

RBC hemolysis assay: To assess the hemolytic activity of a structurally-stabilized peptide described herein, a red blood cell (RBC) hemolysis assay can be used. Human blood samples are centrifuged to isolate RBCs, which are then washed and suspended in phosphate-buffered saline to yield a 1% (v/v) suspension. The suspension is added to serial dilutions of peptide stocks in water in clear round-bottom polypropylene 96-well plates and the plates incubated for 1 hour at 37° C. The plates are then centrifuged and the supernatant isolated to determine the amount of hemoglobin released using a spectrophotometer (570 nm). Percent hemolysis is calculated as: ([Treated Absorbance - Untreated Control Absorbance] x 100)/(1% Triton X-100 Treated Absorbance - Untreated Control Absorbance).

LDH release assay: To assess the release of lactate dehydrogenase (LDH) upon treatment with a structurally-stabilized peptide described herein, an LDH release assay can be used. Cultured cells, including, e.g., cancer cells and HUVEC cells, are plated in 96-well format (2x10⁴ cells per well; including overnight incubation for adherent cells) and then treated with serial dilutions of structurally-stabilized peptides in a final volume of 100 µL and incubated at 37° C. for the indicated time period (e.g., 90 minutes). The plates are spun down at 1500 rpm for 5 minutes at 4° C., and 80 µL of cell culture media is transferred to a clear plate (Corning), incubated with 80 µL of LDH reagent (Roche) for 15 minutes while shaking, and absorbance is measured at 490 nm on a microplate reader (SpectraMax M5 Microplate Reader, Molecular Devices).

### Methods of Making Structurally-Stabilized Peptides

Also provided herein are methods of making structurally-stabilized peptides (e.g., a structurally-stabilized peptide described herein). In some instances, the method comprises (a) providing a peptide described herein, wherein the peptide comprises two or more stapling amino acids (e.g., a peptide comprising an amino acid sequence set forth in any one of SEQ ID NOs: 2-54, or 56-98), and (b) performing a ring-closing metathesis reaction. In some instances, the method further comprises repeating step (b) one to four additional times (i.e., performing a total of 3 to 5 ring-closing metathesis reactions. In some instances, the method comprises: (a) providing a peptide having the sequence set forth in any one of SEQ ID NOs: 2-54, or 56-98, and (b) cross-linking the peptide. In some instances, the method further comprises formulating the structurally-stabilized peptide as a pharmaceutical composition.

In some instances, the peptides produced by the methods of making the structurally-stabilized peptides are the HDMX-specific structurally stabilized peptides such as those set forth in SEQ ID NOs: 5, 7, 8, 10, 13, 21, 23, 24, 27, 29, 32, 34, 37, 38, 40, 42-46, 48-51, 56-59, 61, 62, 64, 67-76, 78, 80, or 86-94, or (SEQ ID NO:84)-napthalene-(SEQ ID NO:238)as well as variants thereof.

In some instances, the peptides produced by the methods of making the structurally-stabilized peptides are the HDM2-specific structurally stabilized peptides such as those set forth in SEQ ID NOs: 3, 11, 17, 20, 30, 31, or 47, as well as variants thereof.

In some instances, the peptides produced by the methods of making the structurally-stabilized peptides are the HDM2 /HDMX dual binding stabilized peptides such as those set forth in SEQ ID NOs: 2, 4, 6, 9, 12, 14-16, 18, 19, 22, 25, 26, 28, 33, 35, 36, 39, 41, 60, 63, 65, 66, 77, 79, and 81, as well as variants thereof. Variants include sequences with 1 to 10 (e.g., 1, 2, 3, 4, 5, 6, 7, 8, 9, 10) substitutions relative to SEQ ID NOs: 2, 4, 6, 9, 12, 14-16, 18, 19, 22, 25, 26, 28, 33, 35, 36, 39, 41, 60, 63, 65, 66, 77, 79, and 81.

Fmoc-based solid-phase peptide synthesis may be used to synthesize the structurally stabilized peptides described herein (e.g., in accordance with reported methods for generating all-hydrocarbon stapled peptides, e.g., Bird, G. H., Crannell, W. C. & Walensky, L. D. Chemical synthesis of hydrocarbon-stapled peptides for protein interaction research and therapeutic targeting. Curr. Protoc. Chem. Biol. 3, 99-117 (2011)). For example, to achieve the various staple lengths, α-methyl, α-alkenyl amino acids may be installed at i, i+4 positions using two (S)-pentenyl alanine residues (S5) and at i, i+7 positions by inserting (R)-octenyl alanine (R8) at the i position and S5 at the i+7 position. For the stapling reaction, Grubbs first-generation ruthenium catalyst dissolved in dichloroethane is added to the resin-bound peptides. To ensure maximal conversion, 3-5 rounds of stapling may be performed. The peptides are then cleaved off of the resin using, e.g., trifluoroacetic acid, precipitated using, e.g., a hexane:ether (1:1) mixture, air dried and purified by, e.g., LC-MS. Peptides may be quantified by amino acid analysis. TFA-HCl exchange may be performed on peptides to be used in animal studies.

### Methods of Use

The disclosure features methods of using any of the compositions (i.e., the structurally stabilized (e.g., stapled or stitched) peptides or pharmaceutical compositions comprising said structurally stabilized peptides) described herein. The methods of using the compositions disclosed herein include methods of treatment of cancer in a human subject. Methods of treatment of cancer in a human subject include administering one or more of a structurally-stabilized (e.g., stapled) peptide or composition comprising structurally stabilized peptides described herein. Also disclosed herein are methods of diagnosing a subject using one of the compositions disclosed herein. For example, disclosed are methods to identify whether a cancer is HDMX-dependent and thus would be receptive to an HDMX-selective inhibitor.

### Therapeutic Uses

Provided herein is a method of treating a cancer (e.g., an HDMX-overexpressing or HDMX-dependent cancer) using a structurally-stabilized (e.g., stapled) peptide described herein. The disclosure features methods of using a structurally-stabilized oncolytic peptide (e.g., any of the structurally-stabilized (e.g., stapled) peptides (or pharmaceutical compositions comprising said structurally-stabilized peptides) described herein) for the prevention and/or treatment of a cancer (e.g. an HDMX-overexpressing or HDMX-dependent cancer) in a subject (e.g., human) in need thereof. The terms “treat” or “treating,” as used herein, refers to alleviating, inhibiting, or ameliorating the disease (e.g., cancer) from which the subject is suffering.

In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide has at least 60% (at least 65%, at least 70%, at least 75%, at least 80%, at least 85%, or at least 90%) sequence identity to the amino acid sequence LTFEEYWAQBTSAA (SEQ ID NO:101) (over the full length of the sequence), and wherein the structurally stabilized peptide comprises (i) substitution of the amino acid corresponding to L26 of SEQ ID NO: 102 with an amino acid other than phenylalanine, isoleucine, norleucine, and leucine, (ii) a substitution of the amino acid corresponding to F19 of SEQ ID NO: 102 with a charged amino acid or alanine, or (iii) a substitution of the amino acid corresponding to W23 of SEQ ID NO : 102 with a charged amino acid or alanine, and wherein the structurally-stabilized peptide is stapled or stitched. In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide comprises the amino acid sequence LTFEEYWAQBTSAA (SEQ ID NO: 101), comprising 2 to 10 amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with an amino acid other than phenylalanine, isoleucine, norleucine, and leucine, (ii) substitution of the amino acid corresponding to F19 of SEQ ID NO: 102 with a charged amino acid or alanine, or (iii) substitution of the amino acid corresponding to W23 of SEQ ID NO : 102 with a charged amino acid or alanine, and wherein the structurally-stabilized peptide is stapled or stitched. In some instances, the structurally-stabilized peptide comprises a substitution of the amino acid corresponding to L26 of SEQ ID NO: 102 with an amino acid other than phenylalanine, isoleucine, norleucine, and leucine. In some instances, the structurally-stabilized peptide comprises a phenylalanine (Phe, F) at the amino acid corresponding to F19 of SEQ ID NO: 102 (i.e., there is no substitution at F19). In some instances, the structurally-stabilized peptide comprises a tryptophan (Trp, W) at the amino acid corresponding to W23 of SEQ ID NO: 102 (i.e., there is no substitution at W23).

In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide has at least 60% (at least 65%, at least 70%, at least 75%, at least 80%, at least 85%, or at least 90%) sequence identity to the amino acid sequence LTFEEYWAQBTSAA (SEQ ID NO:101) (over the full length of the sequence), and wherein the structurally stabilized peptide comprises (i) substitution of the amino acid corresponding to L26 of SEQ ID NO: 102 with a charged amino acid, alanine, a hydrophobic amino acid less hydrophobic than leucine, or a hydrophilic amino acid, (ii) a substitution of the amino acid corresponding to F19 of SEQ ID NO: 102 with a charged amino acid or alanine, or (iii) a substitution of the amino acid corresponding to W23 of SEQ ID NO : 102 with a charged amino acid or alanine, and wherein the structurally-stabilized peptide is stapled or stitched. In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide comprises the amino acid sequence LTFEEYWAQBTSAA (SEQ ID NO: 101), comprising 2 to 10 amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with a charged amino acid, alanine, a hydrophobic amino acid less hydrophobic than leucine, or a hydrophilic amino acid, (ii) substitution of the amino acid corresponding to F19 of SEQ ID NO:102 with a charged amino acid or alanine, or (iii) substitution of the amino acid corresponding to W23 of SEQ ID NO : 102 with a charged amino acid or alanine, and wherein the structurally-stabilized peptide is stapled or stitched. In some instances, the structurally-stabilized peptide comprises a substitution of the amino acid corresponding to L26 of SEQ ID NO: 102 with a charged amino acid, alanine, a hydrophobic amino acid less hydrophobic than leucine, or a hydrophilic amino acid. In some instances, the structurally-stabilized peptide comprises a phenylalanine (Phe, F) at the amino acid corresponding to F19 of SEQ ID NO: 102 (i.e., there is no substitution at F19). In some instances, the structurally-stabilized peptide comprises a tryptophan (Trp, W) at the amino acid corresponding to W23 of SEQ ID NO: 102 (i.e., there is no substitution at W23).

In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide has at least 60% (at least 65%, at least 70%, at least 75%, at least 80%, at least 85%, or at least 90%) sequence identity to the amino acid sequence LSQETFSDLWKLLPEN (SEQ ID NO:99) (over the full length of the sequence), and wherein the structurally stabilized peptide comprises (i) substitution of the amino acid corresponding to L26 of SEQ ID NO: 102 with an amino acid other than phenylalanine, isoleucine, norleucine, and leucine, (ii) a substitution of the amino acid corresponding to F19 of SEQ ID NO: 102 with a charged amino acid or alanine, or (iii) a substitution of the amino acid corresponding to W23 of SEQ ID NO: 102 with a charged amino acid or alanine, wherein the structurally-stabilized peptide is stapled or stitched. In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide comprises the amino acid sequence LSQETFSDLWKLLPEN (SEQ ID NO:99), comprising 2 to 10 amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with an amino acid other than phenylalanine, isoleucine, norleucine, and leucine, (ii) a substitution of the amino acid corresponding to F19 of SEQ ID NO: 102 with a charged amino acid or alanine, or (iii) a substitution of the amino acid corresponding to W23 of SEQ ID NO: 102 with a charged amino acid or alanine, wherein the structurally-stabilized peptide is stapled or stitched. In some instances, the structurally-stabilized peptide comprises a substitution of the amino acid corresponding to L26 of SEQ ID NO: 102 with an amino acid other than phenylalanine, isoleucine, norleucine, and leucine. In some instances, the structurally-stabilized peptide comprises a phenylalanine (Phe, F) at the amino acid corresponding to F19 of SEQ ID NO: 102 (i.e., there is no substitution at F19). In some instances, the structurally-stabilized peptide comprises a tryptophan (Trp, W) at the amino acid corresponding to W23 of SEQ ID NO: 102 (i.e., there is no substitution at W23).

In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide has at least 60% (at least 65%, at least 70%, at least 75%, at least 80%, at least 85%, or at least 90%) sequence identity to the amino acid sequence LSQETFSDLWKLLPEN (SEQ ID NO:99) (over the full length of the sequence), and wherein the structurally stabilized peptide comprises (i) substitution of the amino acid corresponding to L26 of SEQ ID NO: 102 with a charged amino acid, alanine, a hydrophobic amino acid less hydrophobic than leucine, or a hydrophilic amino acid, (ii) a substitution of the amino acid corresponding to F19 of SEQ ID NO: 102 with a charged amino acid or alanine, or (iii) a substitution of the amino acid corresponding to W23 of SEQ ID NO: 102 with a charged amino acid or alanine, wherein the structurally-stabilized peptide is stapled or stitched. In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide comprises the amino acid sequence LSQETFSDLWKLLPEN (SEQ ID NO:99), comprising 2 to 10 amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with a charged amino acid, alanine, a hydrophobic amino acid less hydrophobic than leucine, or a hydrophilic amino acid, (ii) a substitution of the amino acid corresponding to F19 of SEQ ID NO:102 with a charged amino acid or alanine, or (iii) a substitution of the amino acid corresponding to W23 of SEQ ID NO: 102 with a charged amino acid or alanine, wherein the structurally-stabilized peptide is stapled or stitched. In some instances, the structurally-stabilized peptide comprises a substitution of the amino acid corresponding to L26 of SEQ ID NO: 102 with a charged amino acid, alanine, a hydrophobic amino acid less hydrophobic than leucine, or a hydrophilic amino acid. In some instances, the structurally-stabilized peptide comprises a phenylalanine (Phe, F) at the amino acid corresponding to F19 of SEQ ID NO: 102 (i.e., there is no substitution at F19). In some instances, the structurally-stabilized peptide comprises a tryptophan (Trp, W) at the amino acid corresponding to W23 of SEQ ID NO: 102 (i.e., there is no substitution at W23).

In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide has at least 60% (at least 65%, at least 70%, at least 75%, at least 80%, at least 85%, or at least 90%) sequence identity to the amino acid sequence QSQQTFSNLWRLLPQN (SEQ ID NO: 100) (over the full length of the sequence), and wherein the structurally stabilized peptide comprises (i) substitution of the amino acid corresponding to L26 of SEQ ID NO: 102 with an amino acid other than phenylalanine, isoleucine, norleucine, and leucine, (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid or alanine, wherein the structurally-stabilized peptide is stapled or stitched. In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide comprises the amino acid sequence QSQQTFSNLWRLLPQN (SEQ ID NO: 100), comprising 2 to 10 amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with an amino acid other than phenylalanine, isoleucine, norleucine, and leucine, (ii) a substitution of the amino acid corresponding to F19 of SEQ ID NO: 102 with a charged amino acid or alanine, or (iii) a substitution of the amino acid corresponding to W23 of SEQ ID NO: 102 with a charged amino acid or alanine, wherein the structurally-stabilized peptide is stapled or stitched. In some instances, the structurally-stabilized peptide comprises a substitution of the amino acid corresponding to L26 of SEQ ID NO: 102 with an amino acid other than phenylalanine, isoleucine, norleucine, and leucine. In some instances, the structurally-stabilized peptide comprises a phenylalanine (Phe, F) at the amino acid corresponding to F19 of SEQ ID NO: 102 (i.e., there is no substitution at F19). In some instances, the structurally-stabilized peptide comprises a tryptophan (Trp, W) at the amino acid corresponding to W23 of SEQ ID NO: 102 (i.e., there is no substitution at W23).

In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide has at least 60% (at least 65%, at least 70%, at least 75%, at least 80%, at least 85%, or at least 90%) sequence identity to the amino acid sequence QSQQTFSNLWRLLPQN (SEQ ID NO: 100) (over the full length of the sequence), and wherein the structurally stabilized peptide comprises (i) substitution of the amino acid corresponding to L26 of SEQ ID NO: 102 with a charged amino acid, alanine, a hydrophobic amino acid less hydrophobic than leucine, or a hydrophilic amino acid, (ii) substitution of the amino acid corresponding to F19 of SEQ ID NO: 102 with a charged amino acid or alanine, or (iii) substitution of the amino acid corresponding to W23 of SEQ ID NO: 102 with a charged amino acid or alanine, wherein the structurally-stabilized peptide is stapled or stitched. In some instances, the method of treating cancer (e.g., an HDMX-overexpressing or HDMX-dependent cancer) in a subject (e.g., human) in need thereof comprises administering to the subject a therapeutically effective amount of a structurally-stabilized peptide, wherein the structurally-stabilized peptide comprises the amino acid sequence QSQQTFSNLWRLLPQN (SEQ ID NO:100), comprising 2 to 10 amino acid substitutions relative to the amino acid sequence of SEQ ID NO: 101, wherein at least two of the 2 to 10 amino acid substitutions are substitutions with stapling amino acids, wherein one or more of the 2 to 10 amino acid substitutions is: (i) substitution of the amino acid corresponding to L26 of SEQ ID NO: 102 with a charged amino acid, alanine, a hydrophobic amino acid less hydrophobic than leucine, or a hydrophilic amino acid, (ii) a substitution of the amino acid corresponding to F19 of SEQ ID NO:102 with a charged amino acid or alanine, or (iii) a substitution of the amino acid corresponding to W23 of SEQ ID NO: 102 with a charged amino acid or alanine, wherein the structurally-stabilized peptide is stapled or stitched. In some instances, the structurally-stabilized peptide comprises a substitution of the amino acid corresponding to L26 of SEQ ID NO: 102 with a charged amino acid, alanine, a hydrophobic amino acid less hydrophobic than leucine, or a hydrophilic amino acid. In some instances, the structurally-stabilized peptide comprises a phenylalanine (Phe, F) at the amino acid corresponding to F19 of SEQ ID NO: 102 (i.e., there is no substitution at F19). In some instances, the structurally-stabilized peptide comprises a tryptophan (Trp, W) at the amino acid corresponding to W23 of SEQ ID NO: 102 (i.e., there is no substitution at W23).

In certain instances, the structurally-stabilized peptide used in a method of treating cancer is a structurally stabilized peptide described herein. In some instances, the structurally-stabilized peptide is any one of SEQ ID NOs: 1-98. In some instances, the structurally-stabilized peptide is one of the HDMX-specific stabilized peptides such as those set forth in SEQ ID NOs: 5, 7, 8, 10, 13, 21, 23, 24, 27, 29, 32, 34, 37, 38, 40, 42-46, 48-51, 56-59, 61, 62, 64, 67-76, 78, 80, 86-94, or 96, or (SEQ ID NO:84)-napthalene-(SEQ ID NO:238), as well as variants thereof. In some instances, the structurally-stabilized peptide is one of the HDM2-specific stabilized peptides such as those set forth in SEQ ID NO: 47, as well as variants thereof. In some instances, the structurally-stabilized peptide is one of the HDM2 /HDMX dual binding stabilized peptides such as those set forth in SEQ ID NOs: 2-4, 6, 9, 11, 12, 14-20, 22, 25, 26, 28, 30, 31, 33, 35, 36, 39, 41, 60, 63, 65, 66, 77, 79, and 81, as well as variants thereof.

In certain instances, the structurally-stabilized peptide used in a method of treating cancer is a structurally stabilized peptide described herein.

In some instances, the cancer cell is a hematological cancer cell. In some instances, the cancer cell is a leukemia cancer cell. In some instances, the cancer cell is an acute myeloid leukemia cancer cell. In some instances, the cancer cell is a mixed lineage leukemia cancer cell. In some instances, the cancer cell is a lymphoma cancer cell. In certain cases, the lymphoma cell is a histiocytic lymphoma cell. In certain cases, the cancer cell is multiple myeloma cell. In some instances, the methods disclosed herein include treating a subject having cancer by administering a structurally-stabilized peptide that is one of the HDMX-specific stabilized peptides such as those set forth in SEQ ID NOs: 5, 7, 8, 10, 13, 21, 23, 24, 27, 29, 32, 34, 37, 38, 40, 42-46, 48-51, 56-59, 61, 62, 64, 67-76, 78, 80, 86-94, or 96, or (SEQ ID NO:84)-napthalene-(SEQ ID NO:238), as well as variants thereof. In some instances, the structurally-stabilized peptide is one of the HDM2-specific stabilized peptides such as those set forth in SEQ ID NO: 47, as well as variants thereof. In some instances, the structurally-stabilized peptide is one of the HDM2 /HDMX dual binding stabilized peptides such as those set forth in SEQ ID NOs: 2-4, 6, 9, 11, 12, 14-20, 22, 25, 26, 28, 30, 31, 33, 35, 36, 39, 41, 60, 63, 65, 66, 77, 79, and 81, as well as variants thereof.

In some instances, the cancer is pediatric cancer. In some instances, the pediatric cancer is one of acute myeloid leukemia (AML), acute lymphoblastic leukemia (ALL) (including T cell lineage ALL and B cell lineage ALL), Ewing sarcoma, retinoblastoma, neuroblastoma, glioma (including, e.g., diffuse interstitial pontine glioma (DIPG)), medulloblastoma, rhabdomyosarcoma (including, e.g., alveolar rhabdomyosarcoma and embryonal rhabdomyosarcoma), Wilm’s tumor, and malignant rhabdoid tumor (MRT). In some instances, the peptides disclosed herein are also be applied to other forms of pediatric cancer, including other brain tumors, e.g., anaplastic astrocytoma, atypical teratoid rhabdoid tumor (AT/RT), diffuse astrocytoma, ependymoma.

In some instances, the pediatric cancer is a pediatric leukemia. In some instances, the pediatric leukemia is acute myeloid leukemia. In some instances, the pediatric leukemia is acute lymphoblastic leukemia. In some instances, the acute lymphoblastic leukemia is a T cell lineage acute lymphoblastic leukemia or a B cell lineage acute lymphoblastic leukemia. In some instances, the pediatric cancer is Ewing sarcoma. In some instances, the pediatric cancer is selected from the group consisting of retinoblastoma, neuroblastoma, osteosarcoma, a glioma, medulloblastoma, rhabdomyosarcoma, Wilm’s tumor, and a malignant rhabdoid tumor. In some instances, the rhabdomyosarcoma is alveolar or embryonal rhabdomyosarcoma. In some instances, the glioma is a diffuse interstitial pontine glioma. In some instances, the pediatric cancer is a relapsed cancer. In some instances, the pediatric cancer was refractory to one or more previous treatments.

In some instances, the pediatric cancer is a rhabdoid tumor.

In some instances, the pediatric cancer is Ewing sarcoma.

In some instances, the pediatric cancer is diffuse interstitial pontine glioma.

In some insteances, the cancer is a solid cancer (e.g., osteosarcoma or choriocarcinoma).

In certain instances, the structurally-stabilized peptide (e.g., one of the HDMX-specific stabilized peptides such as those set forth in SEQ ID NOs: 5, 7, 8, 10, 13, 21, 23, 24, 27, 29, 32, 34, 37, 38, 40, 42-46, 48-51, 56-59, 61, 62, 64, 67-76, 78, 80, 86-94, or 96, or or (SEQ ID NO:84)-napthalene-(SEQ ID NO:238), as well as variants thereof) used in a method to lyse cancer cells (e.g., hematological cancer cells, e.g., leukemia cells, lymphoma cells, multiple myeloma cells). In certain instances, the structurally-stabilized peptides disclosed herein are capable of specifically lysing hematological cancer cells. In certain instances, the structurally-stabilized peptides disclosed herein are capable of specifically lysing leukemia cells. In certain instances, the structurally-stabilized peptides disclosed herein are capable of specifically lysing lymphoma cells. In certain instances, the structurally-stabilized peptides disclosed herein are capable of specifically lysing multiple myeloma cells. In certain instances, the structurally-stabilized peptides disclosed herein for use in a method of treating cancer or in a method of inhibiting proliferation of a cancer cell are capable of specifically lysing cancer cells of the type of cancer to be treated or of the type of cancer cell for which proliferation is to be inhibited. For example, if the method comprises treating leukemia/lymphoma, the structurally-stabilized peptide (or composition comprising the structurally-stabilized peptide) used in the method is capable of specifically lysing leukemia/lymphoma cells. In another example, if the method comprises inhibiting leukemia/lymphoma cancer cells, the structurally-stabilized peptide (or composition comprising the structurally-stabilized peptide) used in the method is capable of specifically lysing leukemia/lymphoma cells. It follows that a structurally-stabilized peptide that is incapable of specifically lysing cells of a particular cancer type is not used in a method of treating that cancer type or in a method of inhibiting proliferation of a cancer cell of that cancer type. For example, a structurally-stabilized peptide that is incapable of specifically lysing, e.g., breast cancer cells, would not be used in a method of treating breast cancer in a subject in need thereof. As another example, a structurally-stabilized peptide that is incapable of specifically lysing, e.g., breast cancer cells, would not be used in a method of inhibiting proliferation of a breast cancer cell in a subject in need thereof. Methods for determining if a structurally-stabilized peptide is capable of specifically lysing cancer cells are known in the art and described herein.

The structurally-stabilized (e.g., stapled) peptides (or compositions comprising the peptides) described herein can be useful for treating a cancer in a human subject. The peptides (or compositions comprising the peptides) described herein can also be useful for preventing cancer from developing in a human subject. Thus, provided herein are methods of treating a cancer in a human subject in need thereof, comprising administering to the subject a therapeutically effective amount of a structurally-stabilized peptide described herein (or a composition comprising the structurally-stabilized peptide). Also provided herein are methods of preventing a cancer in a human subject in need thereof, comprising administering to the subject a therapeutically effective amount of a structurally-stabilized peptide described herein (or a composition comprising the structurally-stabilized peptide).

In certain instances, the human subject in need thereof is administered any one of constructs 1-92 described in Table 4.

Where the structurally-stabilized peptide is an HDMX-selective peptide, the cancer is an HDMX-overexpressing or HDMX-dependent cancer. In some instances, the cancer (e.g., an HDMX-overexpressing or HDMX-dependent cancer) is a hematologic cancer. In some instances, the cancer (e.g., an HDMX-overexpressing or HDMX-dependent cancer) is a solid cancer. In some instances, the cancer (e.g., an HDMX-overexpressing or HDMX-dependent cancer) is an eosinophilic leukemia. In some instances, the cancer (e.g., an HDMX-overexpressing or HDMX-dependent cancer) is acute myeloid leukemia. In some instances, the cancer (e.g., an HDMX-overexpressing or HDMX-dependent cancer) is osteosarcoma. In some instances, the cancer (e.g., an HDMX-overexpressing or HDMX-dependent cancer) is pediatric cancer.

Where the structurally-stabilized peptide is an HDM2-selective peptide, the cancer is an HDM2-overexpressing or HDM2-dependent cancer. In some instances, the cancer (e.g., an HDM2-overexpressing or HDM2-dependent cancer) is a hematologic cancer. In some instances, the cancer (e.g., an HDM2-overexpressing or HDM2-dependent cancer) is a solid cancer. In some instances, the cancer (e.g., an HDM2-overexpressing or HDM2-dependent cancer) is an eosinophilic leukemia. In some instances, the cancer (e.g., an HDM2-overexpressing or HDM2-dependent cancer) is acute myeloid leukemia. In some instances, the cancer (e.g., an HDM2-overexpressing or HDM2-dependent cancer) is osteosarcoma. In some instances, the cancer (e.g., an HDM2-overexpressing or HDM2-dependent cancer) is pediatric cancer.

Where the structurally-stabilized peptide is an HDMX- and HDM2-selective peptide, the cancer is an HDMX- and/or HDM2-overexpressing or HDMX- and/or HDM2-dependent cancer. In some instances, the cancer (e.g., an HDMX- and/or HDM2-overexpressing or HDMX- and/or HDM2-dependent cancer) is a hematologic cancer. In some instances, the cancer (e.g., an HDMX- and/or HDM2-overexpressing or HDMX- and/or HDM2-dependent cancer) is a solid cancer. In some instances, the cancer (e.g., an HDMX- and/or HDM2-overexpressing or HDMX- and/or HDM2-dependent cancer) is an eosinophilic leukemia. In some instances, the cancer (e.g., an HDMX- and/or HDM2-overexpressing or HDMX- and/or HDM2-dependent cancer) is acute myeloid leukemia. In some instances, the cancer (e.g., an HDMX- and/or HDM2-overexpressing or HDMX- and/or HDM2-dependent cancer) is osteosarcoma. In some instances, the cancer (e.g., an HDMX-overexpressing or HDMX-dependent cancer) is pediatric cancer.

HDMX-overexpressing cancers are known in the art. In some instances, a cancer “overexpresses” HDMX if it expresses HDMX at a level that is at least 1.5-fold, at least 2-fold, at least 3-fold, at least 4-fold, at least 5-fold, at least 6-fold, at least 7-fold, at least 8-fold, at least 9-fold, at least 10-fold, or at least 20-fold higher than a level of HDMX in a non-cancerous control sample of the same cell type. For instance, an HDMX-overexpressing Ewing sarcoma cancer expresses HDMX at a level that is at least 1.5-fold, at least 2-fold, at least 3-fold, at least 4-fold, at least 5-fold, at least 6-fold, at least 7-fold, at least 8-fold, at least 9-fold, at least 10-fold, or at least 20-fold higher than a level of HDMX in a non-cancerous sample of bone tissue.

HDM2-overexpressing cancers are known in the art. In some instances, a cancer “overexpresses” HDM2 if it expresses HDM2 at a level that is at least 1.5-fold, at least 2-fold, at least 3-fold, at least 4-fold, at least 5-fold, at least 6-fold, at least 7-fold, at least 8-fold, at least 9-fold, at least 10-fold, or at least 20-fold higher than a level of HDM2 in a non-cancerous control sample of the same cell type. For instance, an HDM2-overexpressing Ewing sarcoma cancer expresses HDM2 at a level that is at least 1.5-fold, at least 2-fold, at least 3-fold, at least 4-fold, at least 5-fold, at least 6-fold, at least 7-fold, at least 8-fold, at least 9-fold, at least 10-fold, or at least 20-fold higher than a level of HDM2 in a non-cancerous sample of bone tissue.

HDMX-dependent cancers and HDM2-dependent cancers are also known in the art and can be identified according to a method described herein. For instance, depmap.org/portal may be used to identify cancers dependent on HDMX or HDM2. In some instances, a cancer is dependent on HDMX or HDM2 if deletion or inhibition of HDMX or HDM2, respectively, in the cancerous cells kills the cancer.

In general, methods include selecting a subject and administering to the subject an effective amount of one or more of the structurally-stabilized (e.g., stapled) peptide herein, e.g., in or as a pharmaceutical composition, and optionally repeating administration as required for the method (e.g., the prevention or treatment of cancer or the inhibition of proliferation of a cancer cell) and can be administered orally, intravenously or topically. A subject can be selected for treatment based on, e.g., determining that the subject has cancer.

Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject’s disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds (structurally-stabilized peptides) selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Diagnostic Uses

Provided herein is a method of diagnosing a subject as having cancer (e.g., an HDMX-overexpressing or HDMX-dependent cancer) using a structurally-stabilized (e.g., stapled) peptide described herein. The disclosure features methods of using a structurally-stabilized oncolytic peptide (e.g., any of the structurally-stabilized (e.g., stapled) peptides (or pharmaceutical compositions comprising said structurally-stabilized peptides) described herein) to determine whether a subject would be receptive to an HDMX-selective or dual HDM2-HDMX inhibitor.

Moreover, the disclosure additionally provides a method for predicting the efficacy of treatment of an HDMX-selective or dual HDM2-HDMX inhibitor in a subject having cancer. In some instances, the methods include testing a cell of a subject having cancer for the presence of wild-type or functional p53, and predicting that a structurally-stabilized (e.g., stapled) peptide would likely reverse inhibition of p53 activity in the cancer (and thereby treat the cancer) if the cell possesses wild-type or functional p53.

In some instances, the methods include isolating cells (e.g., biopsy; e.g., liquid biopsy) from a subject having cancer. In some instances, the isolated cells are cultured and treated with one or more of the HDMX-selective inhibitors and/or dual HDM2-HDMX inhibitors as disclosed herein. If the HDMX-selective inhibitor produces the same result (e.g., detection of the abundance of p53 or p53-related activity is the same or similar) as cells treated with a dual HDM2-HDMX inhibitor, then this would suggest that the cancer cells are HDMX-dependent. Thus, in some instances, the methods further include administering one or more HDMX-selective inhibitors to the subject whose cells produce the same result (e.g., produces the same abundance of p53 or p53-related activity) as cells treated with a dual HDM2-HDMX inhibitor.

In some instances, the methods include isolating cells (e.g., biopsy; e.g., liquid biopsy) from a subject having cancer. In some instances, the isolated cells are cultured and treated with one or more of the HDMX-selective inhibitors and/or dual HDM2-HDMX inhibitors as disclosed herein. If the dual HDM2-HDMX inhibitor produces the same result (e.g., detection of the abundance of p53 or p53-related activity is the same or similar) as cells treated with a dual HDM2-HDMX inhibitor, then this would suggest that the cancer cells are HDMX-dependent. Thus, in some instances, the methods further include administering one or more HDMX-selective inhibitors to the subject whose cells produce the same result (e.g., produces the same abundance of p53 or p53-related activity) as cells treated with a dual HDM2-HDMX inhibitor.

In some instances, the method can include, if the cancer cell is found to express wild-type or functional p53 (and detectable HDM2 and/or HDMX), administering one or more structurally-stabilized (e.g., stapled) peptides as disclosed herein that target HDM2, HDMX, or both HDM2 and HDMX to the subject with the cancer. In some embodiments, the methods can include developing a personalized treatment regimen for a subject with cancer. Such methods can include, e.g., identifying a subject with cancer cells that are sensitive to one or more structurally-stabilized (e.g., stapled) peptides as disclosed herein and treating the subject with one or more structurally-stabilized (e.g., stapled) peptides as disclosed herein. In some embodiments, the methods can include determining the most appropriate treatment for a subject confirmed to have cancer (e.g., by determining the susceptibility of one or more of the subject’s cancer cells to treatment using the compositions disclosed herein (e.g., in vitro)), developing a treatment regimen for the subject, and optionally administering to the subject a composition in accordance with the treatment regimen.

In some instances, the methods can include, for example, selecting a subject having a cancer; evaluating (e.g., detecting) the expression and/or activity of p53 in the subject’s cancer (e.g., in a cancer cell obtained from the subject (e.g., obtained by biopsy); and, if p53 expression and/or activity is detected, providing the subject with a personalized treatment regimen that includes administering an effective amount of one or more structurally-stabilized (e.g., stapled) peptides as disclosed herein to the subject. In some instances, the one or more structurally-stabilized (e.g., stapled) peptides as disclosed herein are HDMX-selective inhibitors as disclosed herein (e.g., SEQ ID NOs: 5, 7, 8, 10, 13, 21, 23, 24, 27, 29, 32, 34, 37, 38, 40, 42-46, 48-51, 56-59, 61, 62, 64, 67-76, 78, 80, 86-94, or 96, or (SEQ ID NO:84)-napthalene-(SEQ ID NO:238), as well as variants thereof). In some instances, the one or more structurally-stabilized (e.g., stapled) peptides as disclosed herein are a dual HDM2-HDMX inhibitor (e.g., SEQ ID NOs: 2-4, 6, 9, 11, 12, 14-20, 22, 25, 26, 28, 30, 31, 33, 35, 36, 39, 41, 60, 63, 65, 66, 77, 79, and 81, as well as variants thereof).

In some instances, the methods include selecting a subject having cancer; detecting the presence and/or level of a p53-HDMX complex in a sample (e.g., a cancer cell) obtained from the subject (e.g., a cancer cell obtained by biopsy); and, if the p53-HDMX complex is detected, providing the subject with a personalized treatment regimen that includes administering an effective amount of one or more structurally-stabilized (e.g., stapled) peptides as disclosed herein to the subject. In some embodiments, the method includes administering the one or more structurally-stabilized (e.g., stapled) peptides as disclosed herein to the subject under conditions and for a period of time sufficient to treat the subject.

In some instances, the methods include selecting a subject having cancer; detecting the presence and/or level of a p53-HDMX complex in a sample (e.g., a cancer cell) obtained from the subject (e.g., a cancer cell obtained by biopsy) and assessing the level of p53 in the sample to determine if the level or activity of p53 is low (e.g., relative to the level or activity of p53 in a cancer cell that exhibits reduced viability when contacted with one or more structurally-stabilized (e.g., stapled) peptides as disclosed herein). In some embodiments, activity can be assessed by titrating dissociation of HDMX-p53 complexes, as described herein); and, if the p53-HDMX complex is detected and the level of p53 is low, providing the subject with a personalized treatment regimen that includes administering an effective amount of one or more structurally-stabilized (e.g., stapled) peptides as disclosed herein to the subject. In some embodiments, the methods can also include providing the subject with a personalized treatment regimen that further includes administering an effective amount of a composition and/or method for inducing p53 expression and/or activity. In some embodiments, the method includes administering the one or more structurally-stabilized (e.g., stapled) peptides as disclosed herein and, optionally, the composition and/or method for inducing p53 expression and/or activity to the subject under conditions and for a period of time sufficient to treat the subject.

In some instances, the methods include selecting a subject with a cancer that has previously received one or more HDM2-modulating agents (e.g., Nutlin-3), but whose cancer cells were resistant (e.g., partially resistant) to the HDM2-modulating agents (e.g., Nutlin-3); and providing the subject with a personalized treatment regimen that includes administering an effective amount of one or more structurally-stabilized (e.g., stapled) peptides as disclosed herein and, optionally, a composition and/or method for inducing p53 expression and/or activity. In some embodiments, the method includes administering the one or more structurally-stabilized (e.g., stapled) peptides as disclosed herein and, optionally, the composition for inducing p53 expression and/or activity to the subject under conditions and for a period of time sufficient to treat the subject.

In some instances, the assays described herein, alone or in combination, can be used to identify pediatric cancers and/or types of pediatric cancers generally susceptible to, or likely to be susceptible to, treatment with any of the peptides described herein, including HDM2-specific peptides. For example, we have discovered that a large subset of pediatric cancers are unexpectedly susceptible to treatment with these peptides. These include, but are not limited to, e.g., the following pediatric cancers, including their presenting, relapsed, and/or refractory subtypes: acute myeloid leukemia (AML), acute lymphoblastic leukemia (ALL) (including T cell lineage ALL and B cell lineage ALL), Ewing sarcoma, retinoblastoma, neuroblastoma, glioma (including, e.g., diffuse interstitial pontine glioma (DIPG)), medulloblastoma, rhabdomyosarcoma (including, e.g., alveolar rhabdomyosarcoma and embryonal rhabdomyosarcoma), Wilm’s tumor, and malignant rhabdoid tumor (MRT). The compounds, assays, and methods of the document can also be applied to other forms of pediatric cancer, including other brain tumors, e.g., anaplastic astrocytoma, atypical teratoid rhabdoid tumor (AT/RT), diffuse astrocytoma, ependymoma, glioblastoma multiformae (GBM), gliomas, myeloid leukemias, oligodendroma, pilocytic astrocytoma, and primitive neuroectodermal tumor (PNET).

### Pharmaceutical Compositions

One or more of any of the structurally-stabilized (e.g., stapled) peptides described herein can be formulated for use as or in pharmaceutical compositions. The pharmaceutical compositions may be used in the methods of use described herein (see above). In certain instances, the pharmaceutical composition comprises a structurally-stabilized (e.g., stapled) peptide comprising or consisting of an amino acid sequence that is identical to an amino acid sequence set forth in any one of FIG. 3A, FIG. 3B, FIG. 4A, or FIG. 4B. In certain instances, the pharmaceutical composition comprises a structurally-stabilized (e.g., stapled) peptide comprising or consisting of an amino acid sequence that is identical to an amino acid sequence set forth in any one of FIG. 3A, FIG. 3B, FIG. 4A, or FIG. 4B, except for 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide comprising or consisting of an amino acid sequence that is identical to an amino acid sequence set forth in any one of FIG. 3A, FIG. 3B, FIG. 4A, or FIG. 4B, except for 1 to 10, 1 to 9, 1 to 8, 1 to 7, 1 to 6, 1 to 5, 1 to 4, 1 to 3, 1 to 2, or 1 amino acid substitution, insertion, or deletion. In certain instances, the pharmaceutical composition comprises a structurally-stabilized (e.g., stapled) peptide comprising or consisting of any one of constructs 1-92 of Formula (I) described in Table 4, except for 1 to 10, 1 to 9, 1 to 8, 1 to 7, 1 to 6, 1 to 5, 1 to 4, 1 to 3, 1 to 2, or 1 amino acid substitution, insertion, or deletion. Such compositions can be formulated or adapted for administration to a subject via any route, e.g., any route approved by the Food and Drug Administration (FDA). Exemplary methods are described in the FDA’s CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal administration, and/or topical administration (including topical (e.g., nasal) sprays and/or solutions).

In some instances, pharmaceutical compositions can include an effective amount of one or more structurally-stabilized (e.g., stapled) peptides. The terms “effective amount” and “effective to treat,” as used herein, refer to an amount or a concentration of one or more structurally-stabilized (e.g., stapled) peptides or a pharmaceutical composition described herein utilized for a period of time (including acute or chronic administration and periodic or continuous administration) that is effective within the context of its administration for causing an intended effect or physiological outcome (e.g., treatment of cancer).

Pharmaceutical compositions of this disclosure can include one or more structurally-stabilized (e.g., stapled) peptides described herein and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a structurally-stabilized peptide of this disclosure, and which does not destroy the pharmacological activity thereof and is nontoxic when administered in doses sufficient to deliver a therapeutic amount of the compound.

The pharmaceutical compositions of this disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

In some instances, one or more structurally-stabilized (e.g., stapled) peptides disclosed herein can be conjugated, for example, to a carrier protein. Such conjugated compositions can be monovalent or multivalent. For example, conjugated compositions can include one structurally-stabilized (e.g., stapled) peptide disclosed herein conjugated to a carrier protein. Alternatively, conjugated compositions can include two or more structurally-stabilized (e.g., stapled) peptides disclosed herein conjugated to a carrier.

As used herein, when two entities are “conjugated” to one another they are linked by a direct or indirect covalent or non-covalent interaction. In certain instances, the association is covalent. In other instances, the association is non-covalent. Non-covalent interactions include hydrogen bonding, van der Waals interactions, hydrophobic interactions, magnetic interactions, electrostatic interactions, etc. An indirect covalent interaction occurs when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Natl. Acad. Sci. USA 88:7175-7179, 1991; Li et al., Infect. Immun. 57:3823-3827, 1989; Szu et al., Infect. Immun. 59:4555-4561,1991; Szu et al., J. Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

### Exemplary Structurally-Stabilized Peptides and Methods of Use

In certain instances, the disclosure features a peptide that selectively binds to HDMX over HDM2 (e.g., binds to HDMX with at least 1.5-fold, at least 2-fold, at least 3-fold, at least 4-fold, at least 5-fold, or at least 10-fold greater affinity than to HDM2). In some instances, the peptide comprises SEQ ID NO: 59, 80, 86, 96, or 97. In some cases, the peptide is a variant of these peptides. For example, in some instances, the peptide comprises the amino acid sequence of SEQ ID NO: 59 with 1 to 7 (e.g., 1, 2, 3, 4, 5, 6, 7) amino acid substitutions. In some instances, the peptide comprises the amino acid sequence of SEQ ID NO: 80 with 1 to 7 (e.g., 1, 2, 3, 4, 5, 6, 7) amino acid substitutions. In some instances, the peptide comprises the amino acid sequence of SEQ ID NO:86 with 1 to 7 (e.g., 1, 2, 3, 4, 5, 6, 7) amino acid substitutions. In some instances, the peptide comprises the amino acid sequence of SEQ ID NO:96 with 1 to 7 (e.g., 1, 2, 3, 4, 5, 6, 7) amino acid substitutions. In some instances, the peptide comprises the amino acid sequence of SEQ ID NO: 97 with 1 to 7 (e.g., 1, 2, 3, 4, 5, 6, 7) amino acid substitutions. The substitutions in these variants are sometimes on the non-interacting face of the peptide. In some cases, all of these variants have Phe19 and Trp23 of wild type p53. Each of these variants also selectively bind to HDMX over HDM2 (e.g., binds to HDMX with at least 1.5-fold, at least 2-fold, at least 3-fold, at least 4-fold, at least 5-fold, or at least 10-fold greater affinity than to HDM2).

In some instances, the disclosure provides a stapled peptide comprising or consisting of the amino acid sequence of any one of SEQ ID NO: 59, 80, 86, 96, or 97 or comprising 1 to 13 (i.e., 1, 2, 3, 4, 5, 6, 7, 8, 9, 10, 11, 12, or 13) amino acid substitutions, deletions and/or insertions therein. Each of these variants also selectively bind to HDMX over HDM2 (e.g., binds to HDMX with at least 1.5-fold, at least 2-fold, at least 3-fold, at least 4-fold, at least 5-fold, or at least 10-fold greater affinity than to HDM2). In some cases, all of these variants have Phe19 and Trp23 of wild type p53.

In certain instances, disclosed herein are stapled peptides that are variants of the amino acid sequence of ATSP-7041 (SEQ ID NO:55) that selectively bind to HDMX over HDM2. In some instances, the stapled peptide selectively binds to HDMX over HDM2 and comprises the amino acid sequence LTF8EYWAQBXSAA (SEQ ID NO:55), wherein each of 8 and X is independently a stapling amino acid and B is cyclobutylalanine, with 1 to 10 (1, 2, 3, 4, 5, 6, 7, 8, 9, or 10) amino acid substitutions relative to the amino acid sequence of SEQ ID NO:55, wherein the 1 to 10 amino acid substitutions are not at positions 4 or 11 of SEQ ID NO:55. In some instances, position 4 of SEQ ID NO:55 is (S)-α-(4′-pentenyl)Alanine and position 11 of SEQ ID NO:55 is (R)-α-(7′-octenyl)Alanine. In some instances, position 4 of SEQ ID NO:55 is (R)-α-(7′-octenyl)Alanine and position 11 of SEQ ID NO:55 is (S)-α-(4′-pentenyl)Alanine. In some instances, position 10 of SEQ ID NO:55 is substituted with another amino acid. In one case, the substitution is to glutamic acid. In another case, the substitution is to E(OMe). In yet another case, the substitution is to 5-fluoronorvaline. In some instances, these peptides include 1 to 10 additional substitutions on the non-interacting and/or interacting face of the helix. The amino acids of the interacting face are at positions 1, 3, 4, 6, 7, 10, and 11. In some cases, the amino acids of the interacting face are not substituted. In some cases 1 to 4, 1 to 3, 1 to 2, or 1 amino acids of the interacting face are substituted (e.g., conservative amino acid substitutions). The amino acids of the non-interacting face are at positions 2, 5, 8, 9, 12, 13, and 14. In some cases, 1 to 7, 1 to 6, 1 to 5, 1 to 4, 1 to 3, 1 to 2, or 1 of the amino acids of the non-interacting face are substituted. In some cases the substitutions are non-conservative amino acid substitutions. In other cases, the substitutions are conservative amino acid substitutions. In some cases, the substitutions include both conservative and non-conservative amino acid substitutions. In some cases, all of these variants have Phe19 and Trp23 of wild type p53.

In some instances, position 4 of SEQ ID NO:55 includes a first stapling amino acid. In some instances, position 11 of SEQ ID NO:55 includes a second stapling amino acid. In some instances, the peptide disclosed herein comprises LTF8EYWAQEXSAA (SEQ ID NO:59) or a variant thereof, wherein 8 and X are stapling amino acids. In some instances, the peptide disclosed herein comprises LTF8EYWAQ#XSAA (SEQ ID NO:80) or a variant thereof, wherein 8 and X are stapling amino acids and # is E(OMe). In some instances, the peptide disclosed herein comprises LTF8EYWAQfXSAA (SEQ ID NO:86), wherein 8 and X are stapling amino acids and f is 5-fluoronorvaline.

In certain instances, disclosed herein are stapled peptides that are variants of the amino acid sequence of SAH-p53-8 (SEQ ID NO:95) that selectively bind to HDMX over HDM2. In some instances, the structurally-stabilized peptide selectively binds to HDMX over HDM2 and comprises the amino acid sequence QSQQTF8NLWRLLXQN (SEQ ID NO:95), wherein each of 8 and X is independently a stapling amino acid, with 1 to 10 (1, 2, 3, 4, 5, 6, 7, 8, 9, or 10) amino acid substitutions relative to the amino acid sequence of SEQ ID NO:95, wherein the 1 to 10 amino acid substitutions are not at positions 7 or 14 of SEQ ID NO:55. In some instances, position 7 of SEQ ID NO:95 is (S)-α-(4′-pentenyl)Alanine and position 14 of SEQ ID NO:95 is (R)-α-(7′-octenyl)Alanine. In some instances, position 7 of SEQ ID NO:95 is (R)-α-(7′-octenyl)Alanine and position 14 of SEQ ID NO:95 is (S)-α-(4′-pentenyl)Alanine. In some instances, position 13 of SEQ ID NO:95 is substituted with another amino acid. In one case, the substitution is to glutamic acid. In another case, the substitution is to E(OMe). In some instances, these peptides include 1 to 10 additional substitutions on the non-interacting and/or interacting face of the helix. The amino acids of the interacting face are at positions 4, 6, 7, 9, 10, 12, 13, 15, and 16. In some cases, the amino acids of the interacting face are not substituted. In some cases 1 to 5, 1 to 4, 1 to 3, 1 to 2, or 1 amino acids of the interacting face are substituted (e.g., conservative amino acid substitutions). The amino acids of the non-interacting face are at positions 1, 2, 3, 5, 8, 11, and 14. In some cases, 1 to 7, 1 to 6, 1 to 5, 1 to 4, 1 to 3, 1 to 2, or 1 of the amino acids of the non-interacting face are substituted. In some cases the substitutions are non-conservative amino acid substitutions. In other cases, the substitutions are conservative amino acid substitutions. In some cases, the substitutions include both conservative and non-conservative amino acid substitutions. In some cases, all of these variants have Phe19 and Trp23 of wild type p53.

In some instances, position 4 of SEQ ID NO: 95 includes a first stapling amino ac id. In some instances, position 7 of SEQ ID NO: 55 includes a second stapling amino acid. In some instances, the peptide disclosed herein includes QSQQTF8NLWRLEXQN (SEQ ID NO: 96) or a variant thereof, wherein 8 and X are stapling amino acids. In some instances, the peptide disclosed herein includes QSQQTF8NLWRL#XQN (SEQ ID NO: 97) or a variant thereof, wherein 8 and X are stapling amino acids and # is E(OMe).

In certain instances, the peptide of any one of SEQ ID NOs.: 59, 80, 86, 96, and 97, as well as variants thereof, may contain one or more (e.g., 1, 2, 3, 4, 5, 6, 7, 8, 9, 10) deletions from or additions to the N- and/or C-terminus of the peptide. For example, the peptide comprising the amino acid sequence of SEQ ID NOs.: 59, 80, 86, 96, or 97, as well as variants thereof may be at least 8 amino acids in length but less than 50 (e.g., 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8) amino acids in length. In certain instances, the peptide having any one of SEQ ID NOs.: 59, 80, 86, 96, and 97, as well as variants thereof, are 8-50 amino acids in length. In certain instances, the peptide having any one of SEQ ID NOs.: 59, 80, 86, 96, and 97, as well as variants thereof, are 8-40 amino acids in length. In certain instances, the peptide having any one of SEQ ID NOs.: 59, 80, 86, 96, and 97, as well as variants thereof, are 8-30 amino acids in length. In certain instances, the peptide having any one of SEQ ID NOs.: 59, 80, 86, 96, and 97, as well as variants thereof, are 8-20 amino acids in length. In certain instances, the peptide having any one of SEQ ID NOs.: 59, 80, 86, 96, and 97, as well as variants thereof, are 8-25 amino acids in length. In certain instances, the peptide having any one of SEQ ID NOs.: 59, 80, 86, 96, and 97, as well as variants thereof, are 8-16 amino acids in length. In certain instances, the peptide having any one of SEQ ID NOs.: 59, 80, 86, 96, and 97, as well as variants thereof, are 8-14 amino acids in length. In some cases, all of these peptides have Phe19 and Trp23 of wild type p53.

Also provided herein are methods of making structurally-stabilized peptides (e.g., a structurally-stabilized peptide described herein). In some instances, the method comprises (a) providing a peptide having any one of SEQ ID NOs.: 59, 80, 86, 96, and 97, or variants thereof, and (b) cross-linking the peptide (e.g., by performing a ring-closing metathesis reaction). In some instances, the method further comprises formulating the structurally-stabilized peptide as a pharmaceutical composition.

Also provided herein is a method of treating a cancer (e.g., an HDMX-overexpressing or HDMX-dependent cancer) using a structurally-stabilized (e.g., stapled) peptide having any one of SEQ ID NOs.: 59, 80, 86, 96, and 97, or a variant thereof. The disclosure features methods of using the structurally-stabilized peptide (e.g., any of the structurally-stabilized (e.g., stapled) peptides (or pharmaceutical compositions comprising said structurally-stabilized peptides having any one of SEQ ID NOs.: 59, 80, 86, 96, and 97, or variants thereof) for the prevention and/or treatment of a cancer (e.g. an HDMX-overexpressing or HDMX-dependent cancer) in a subject (e.g., human) in need thereof. In some cases, all of these variants of SEQ ID NOs.: 59, 80, 86, 96, and 97 have Phe19 and Trp23 of wild type p53. In certain cases, the structurally-stabilized peptide has at least 60% (at least 65%, at least 70%, at least 75%, at least 80%, at least 85%, at least 90% or at least 95%) sequence identity to the HDMX interacting face amino acid sequence of any one of SEQ ID NOs.: 59, 80, 86, 96, and 97. In some cases, all of these variants of SEQ ID NOs.: 59, 80, 86, 96, and 97 have Phe19 and Trp23 of wild type p53 and include 1 to 7, 1to 6, 1to 5, 1to 4, 1to 3, 1 to 2, or 1 amino acid substitution on the non-interacting face of the helix (i.e., the face that does not interact with HDMX). In some cases, the amino acid substitution(s) are non-conservative. In other cases, the amino acid substitution(s) are conservative. In yet other cases, the amino acid substitution(s) include both conservative and non-conservative changes.

In some instances, the cancer is a pediatric cancer. In some instances, the pediatric cancer is one of acute myeloid leukemia (AML), acute lymphoblastic leukemia (ALL) (including T cell lineage ALL and B cell lineage ALL), Ewing sarcoma, retinoblastoma, neuroblastoma, glioma (including, e.g., diffuse interstitial pontine glioma (DIPG)), medulloblastoma, rhabdomyosarcoma (including, e.g., alveolar rhabdomyosarcoma and embryonal rhabdomyosarcoma), Wilm’s tumor, and malignant rhabdoid tumor (MRT). In some instances, the peptides disclosed herein are also be applied to other forms of pediatric cancer, including other brain tumors, e.g., anaplastic astrocytoma, atypical teratoid rhabdoid tumor (AT/RT), diffuse astrocytoma, ependymoma. In some instances, the pediatric leukemia is acute myeloid leukemia. In some instances, the pediatric leukemia is acute lymphoblastic leukemia. In some instances, the acute lymphoblastic leukemia is a T cell lineage acute lymphoblastic leukemia or a B cell lineage acute lymphoblastic leukemia. In some instances, the pediatric cancer is Ewing sarcoma. In some instances, the pediatric cancer is selected from the group consisting of retinoblastoma, neuroblastoma, osteosarcoma, a glioma, medulloblastoma, rhabdomyosarcoma, Wilm’s tumor, and a malignant rhabdoid tumor. In some instances, the rhabdomyosarcoma is alveolar or embryonal rhabdomyosarcoma. In some instances, the glioma is a diffuse interstitial pontine glioma. In some instances, the pediatric cancer is a relapsed cancer. In some instances, the pediatric cancer is one that was refractory to one or more previous treatments.

Also provided herein is a method of diagnosing a subject as having cancer (e.g., an HDMX-overexpressing or HDMX-dependent cancer) using a structurally-stabilized (e.g., stapled) peptide having any one of SEQ ID NOs.: 59, 80, 86, 96, and 97, or a variant thereof. The disclosure features methods of using a structurally-stabilized peptide (e.g., any of the structurally-stabilized (e.g., stapled) peptides (or pharmaceutical compositions comprising said structurally-stabilized peptides)) having any one of SEQ ID NOs.: 59, 80, 86, 96, and 97, as well as variants thereof to determine whether a subject would be receptive to an HDMX-selective or dual HDM2-HDMX inhibitor.

Moreover, the disclosure additionally provides a method for predicting the efficacy of treatment of an HDMX-selective or dual HDM2-HDMX inhibitor in a subject having cancer. In some instances, the methods include testing a cell of a subject having cancer for the presence of wild-type or functional p53, and predicting that a structurally-stabilized (e.g., stapled) peptide having any one of SEQ ID NOs.: 59, 80, 86, 96, and 97, or a variant thereof, would likely reverse inhibition of p53 activity in the cancer (and thereby treat the cancer) if the cell possesses wild-type or functional p53. In some instances, the method include isolating cells (e.g., biopsy; e.g., liquid biopsy) from a subject having cancer. In some instances, the isolated cells are cultured and an efficacy of treatment is tested by administering a structurally-stabilized (e.g., stapled) peptide having any one of SEQ ID NOs.: 59, 80, 86, 96, and 97, or a variant thereof. In some instances, the methods can include, for example, selecting a subject having a cancer; evaluating (e.g., detecting) the expression and/or activity of p53 in the subject’s cancer (e.g., in a cancer cell obtained from the subject (e.g., obtained by biopsy); and, if p53 expression and/or activity is detected, providing the subject with a personalized treatment regimen that includes administering an effective amount of one or more structurally-stabilized (e.g., stapled) peptides having any one of SEQ ID NOs.: 59, 80, 86, 96, and 97, as well as variants thereof, to the subject.

### EXAMPLES

The following examples are provided to better illustrate the claimed subject matter and are not to be interpreted as limiting the scope of the disclosure. To the extent that specific materials are mentioned, it is merely for purposes of illustration and is not intended to limit the disclosure. One skilled in the art can develop equivalent means or reactants without the exercise of inventive capacity and without departing from the scope of the disclosure.

### Example 1: Synthesis and Competitive HDM2/HDMX Binding of Stapled P53 Peptide Mutational Libraries

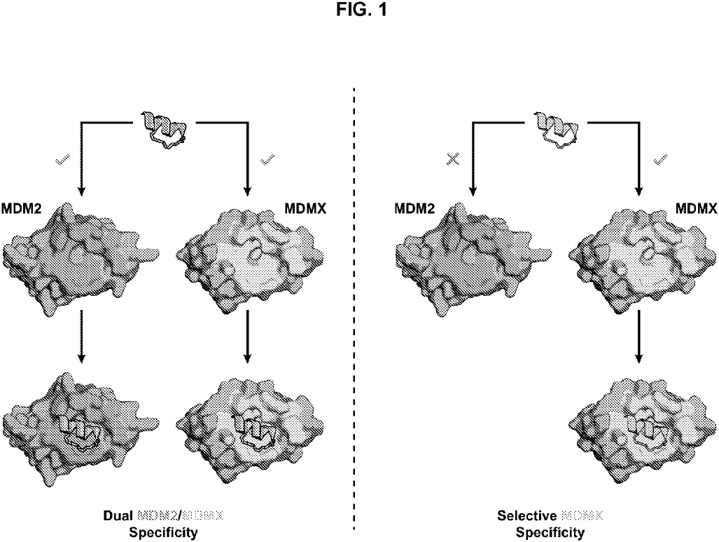

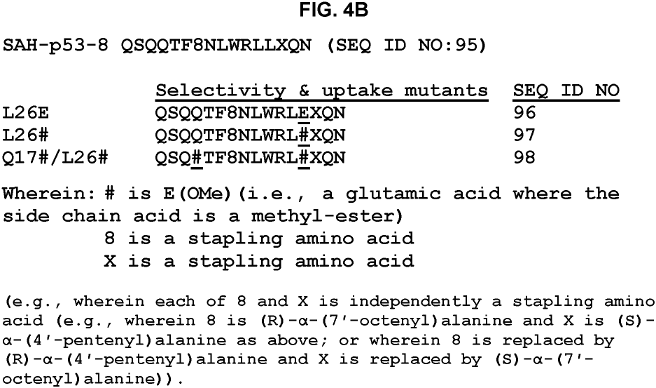

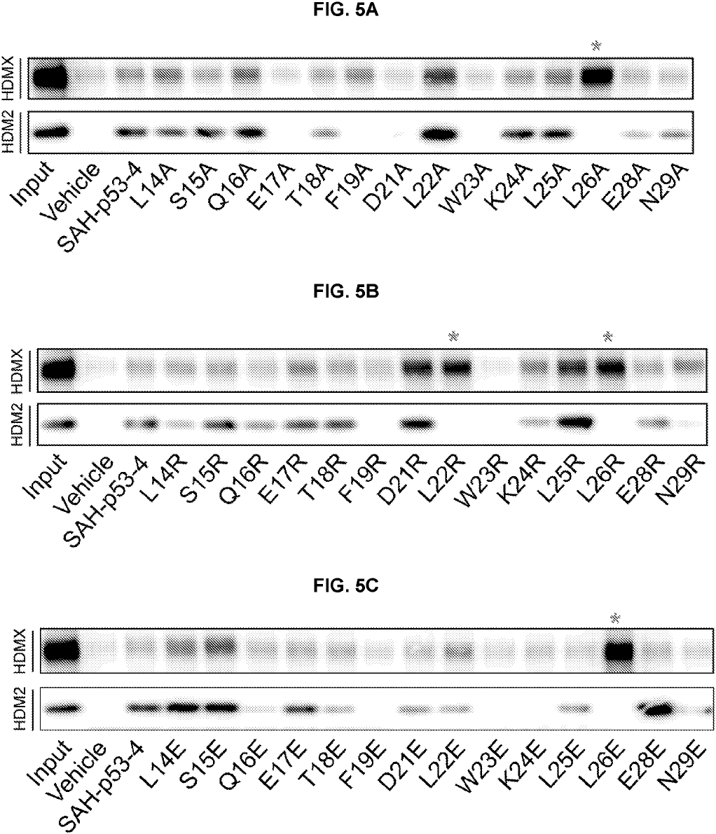

In order to develop an HDMX-selective inhibitor from a naturally dual HDM2/HDMX targeting stapled p53 peptide (FIG. 1), a series of mutational libraries of SAH-p53-4 (FIG. 3A and FIG. 3B), ATSP-7041 (FIG. 4A), and SAH-p53-8 (FIG. 4B) were generated for screening in competitive binding analyses. For these studies, biotinylated peptides were incubated with a 1:1 mixture of recombinant HDM2 (amino acids 17-125 of SEQ ID NO: 103) and HDMX (amino acids 1-490 of SEQ ID NO: 104), followed by streptavidin pull-down and HDM2 and HDMX Western analyses. Whereas the majority of stapled peptide constructs retained dual HDM2/HDMX binding activity, a rare few peptides showed striking HDMX binding selectivity, as exemplified by the L26A (FIG. 5A), L22R and L26R (FIG. 5B), and L26E mutants (FIG. 5C) of SAH-p53-4.

To further validate the qualitative competitive binding results and identify the most potent HDMX-selective stapled p53 peptide among the candidates, quantitative, direct-binding analyses were performed. First, we applied isothermal titration calorimetry (ITC) to measure and compare the binding affinities of SAH-p53-4 wild-type and L26A, L22R, L26R, and L26E mutant peptides for HDMX, using the humanized zebrafish HDMX protein (amino acids 15-106, L46V/V95L; referred to as zHDMX). We chose zHDMX for these studies because it can be produced in high yield, has been successfully applied in prior structural determinations (Chang et al., 2013), and is comparable to that portion of recombinant HDM2 protein used in our competitive binding analyses. Of the SAH-p53-4 peptides tested, the L26E mutant retained the highest binding affinity for zHDMX (Kd, 43.3 nM) as compared to the wild-type peptide (Kd, 37.4 nM), with the L22R, L26A, and L26R mutants showing progressively weaker binding activity (51.1, 92.6, and 151.8 nM, respectively) (FIGS. 6A-E, top and FIG. 6F) . We confirmed this binding hierarchy by an orthogonal approach, namely fluorescence polarization (FP) binding analysis using the corresponding FITC-labeled SAH-p53-4 peptides (FIGS. 6A-E, bottom, and FIG. 6F). Having identified SAH-p53-4 L26E as a lead peptide and to confirm HDMX-selectivity, we then applied ITC and FP to measure and compare its binding affinity for zHDMX and HDM2. Whereas wild-type and L26E-mutant SAH-p53-4 showed comparable binding affinity for zHDMX, L26E mutagenesis caused a 12-fold decrease in binding affinity for HDM2, as measured by both FP (FIGS. 7A-7B) and ITC (FIGS. 7C-7D).

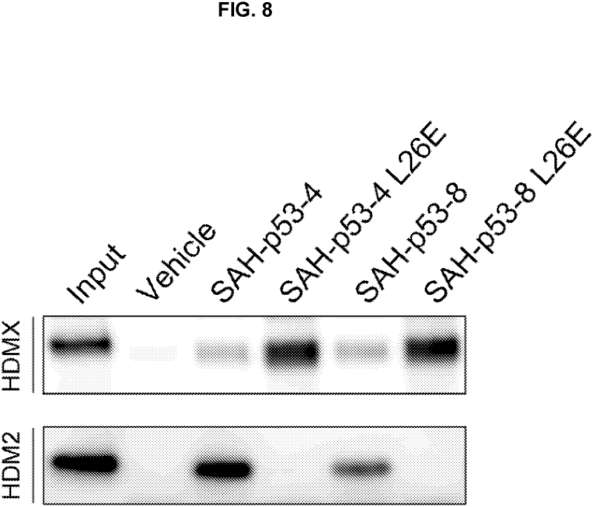

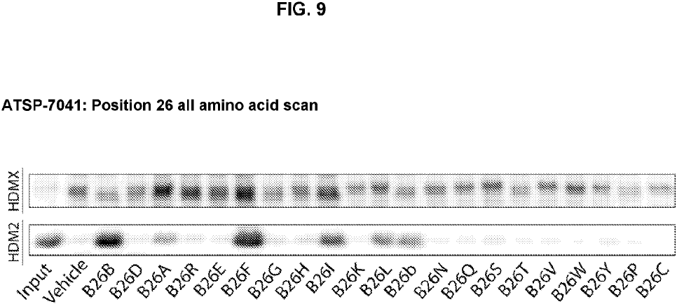

The L26E specificity determinant, installed into the SAH-p53-8 peptide (FIG. 4B) likewise showed exquisite binding selectivity for HDMX (FIG. 8). Replacing the corresponding cyclobutyl alanine (B) in position 26 of ATSP-7041 with each natural amino acid or norleucine, further revealed that Ala, Arg, and Glu mutations yielded the most HDMX-specific ATSP-7041 compounds, whereas mutations that restored hydrophobicity, such as with Phe, Ile, Leu, or NorLeu restored HDM2 binding and thus dual HDM2/HDXM-targeting activity (FIG. 9). These data further suggested that HDMX is significantly more tolerant of diverse mutations at position 26 as compared to HDM2, resulting in the observed HDMX specificity of select compounds.

### Example 2: Structural Basis for HDMX-selectivity of Stapled P53 Peptide Mutants Bearing a Glutamic Acid Residue at Position 26

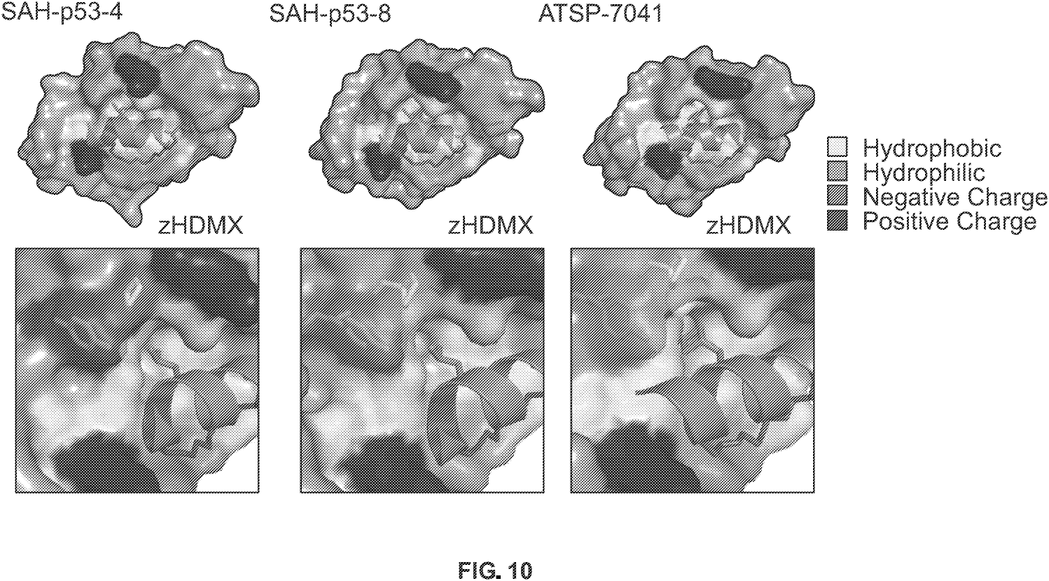

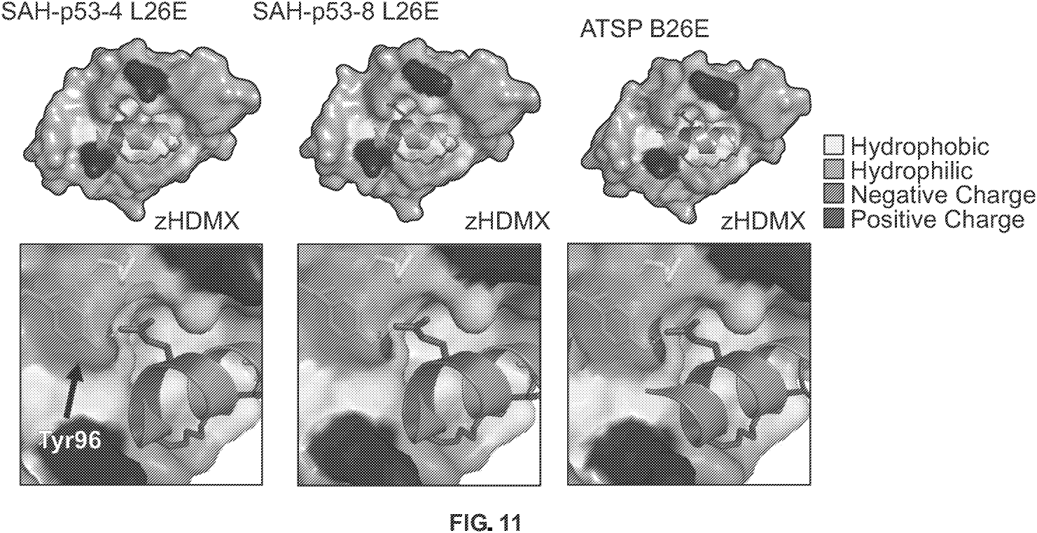

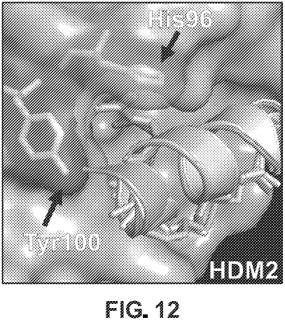

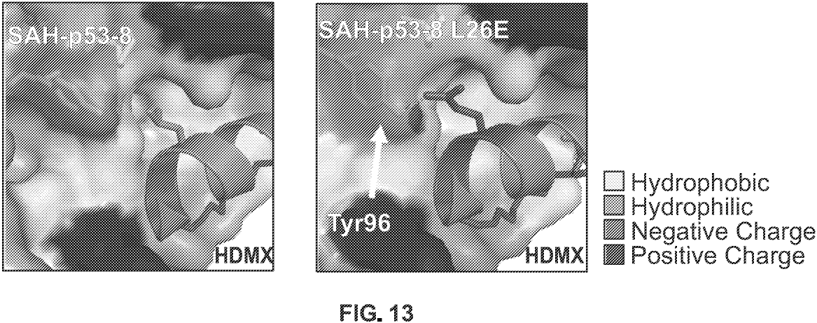

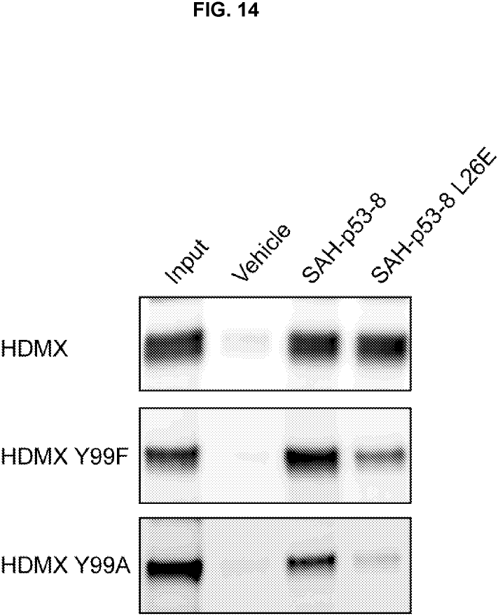

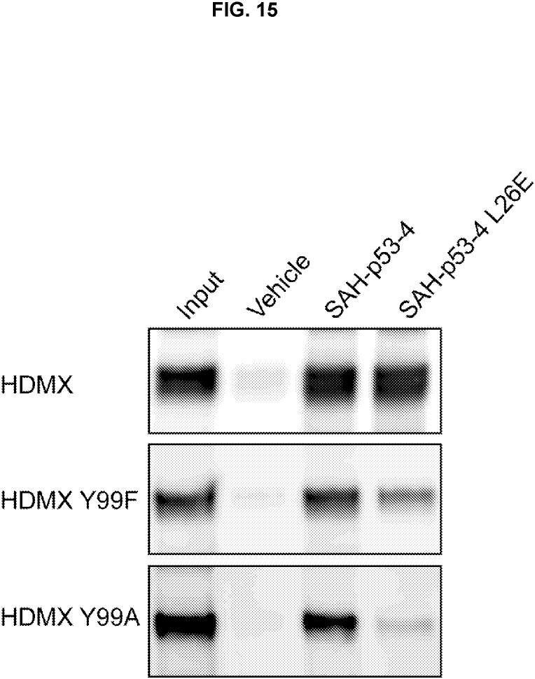

A series of crystal structures were solved to investigate the molecular basis for the HDMX specificity conferred by L26 mutagenesis. L26 naturally engages a deep hydrophobic pocket in the HDM2 and HDMX groove that naturally binds the p53 transactivation domain helix, as shown by the crystal structures of the complexes between HDM2 and SAH-p53-8 (Baek et al., J Am Chem Soc, 2012; PDB ID 3V3B), and between HDMX and SAH-p53-4 (FIG. 10, left, PDB ID 6V4H), SAH-p53-8 (FIG. 10, middle, PDB ID 6V4E), and ATSP-7041 (FIG. 10, right) (Chang et al., Proc Natl Acad Sci, 2013; PDB ID 4N5T). However, upon L26E (SAH compounds) or B26E (ATSP-7041) mutagenesis, the HDMX pocket is capable of accommodating the hydrophobic-to-acidic residue change through engagement of glutamate by Y96 (humanized zebrafish HDMX (amino acids 15-106, L46V/V95L), denoted as zHDMX (LPGEGTQVHPRAPLLQILKVAGAQEEVFTVKEVMHYLGQYIMMKQLYDKQR QHIVHCHDDPLGELLEVGSFSVKNPSPLYEMLKRNLVIL (SEQ ID NO:233)) ; Y99 in HDMX), as demonstrated by the crystal structure of the SAH-p53-4 L26E/zHDMX (FIG. 11, left, PDB ID 6V4F), SAH-p53-8/zHDMX (FIG. 11, middle, PDB ID 6V4G), and ATSP-7041 B26E/zHDMX (FIG. 11, right). Interestingly, an overlay of stapled p53 peptides bearing the glutamate residue onto the structure of the wild-type stapled p53 peptide/HDM2 interaction demonstrates that (1) the analogous tyrosine in HDM2 (Y100) is predicted to be located too distal from the interaction site to engage the installed glutamate (and instead interacts with N29 of the peptide [not shown]) and (2) H96 of the HDM2 binding pocket is predicted to clash with the L26E mutant peptide, providing a potential explanation for the incapability of L26E-mutant stapled p53 peptides to interact with HDM2 (FIG. 12), whereas the HDMX pocket is able to provide an alternative means for peptide engagement by replacing a hydrophobic (FIG. 13, left) with a hydrophilic Y-based (FIG. 13, right) interaction. To evaluate this hypothesis, the influence of Y99F and Y99A mutagenesis was tested in the context of full-length HDMX on binding to both wild-type and L26E-mutant forms of SAH-p53-8 and SAH-p53-4 peptides. In both cases, wild-type SAH-p53-8 (FIG. 14) and SAH-p53-4 (FIG. 15) peptides retained interaction with the Y99F and Y99A mutants of full-length HDMX, whereas the SAH-p53-8 L26E (FIG. 14) and SAH-p53-4 L26E (FIG. 15) mutants were significantly impaired. It is especially striking that only removing the hydroxyl group from Y99 upon mutation to F99, markedly reduced binding to the L26E-mutant SAH-p53-8 and SAH-p53-4 peptides, consistent with selective elimination of the hydrogen-bonding potential of Y99 with the installed glutamate at position 26. These data highlight that this example has discovered an explicit binding determinant for selective stapled p53 peptide engagement of HDMX, which otherwise precludes HDM2 binding, based on the unique interaction topography of the L26E mutant peptides with HDMX involving L26E (stapled p53 peptide) and Y99 (HDMX).

In sum, this data shows that HDMX broadly tolerates mutation at this position (L26) - including positively and negatively charged residues, and less hydrophobic residues than L, such as A, but not for example another bulky hydrophobic residue such as F or I, which enables retention of HDM2 binding activity.

### Example 3: The Recombinant HDMX Selectivity Conferred by Installing Glutamate at Position 26 of Stapled p53 Peptides Translates to the Native HDM2 and HDMX Proteins From Cells

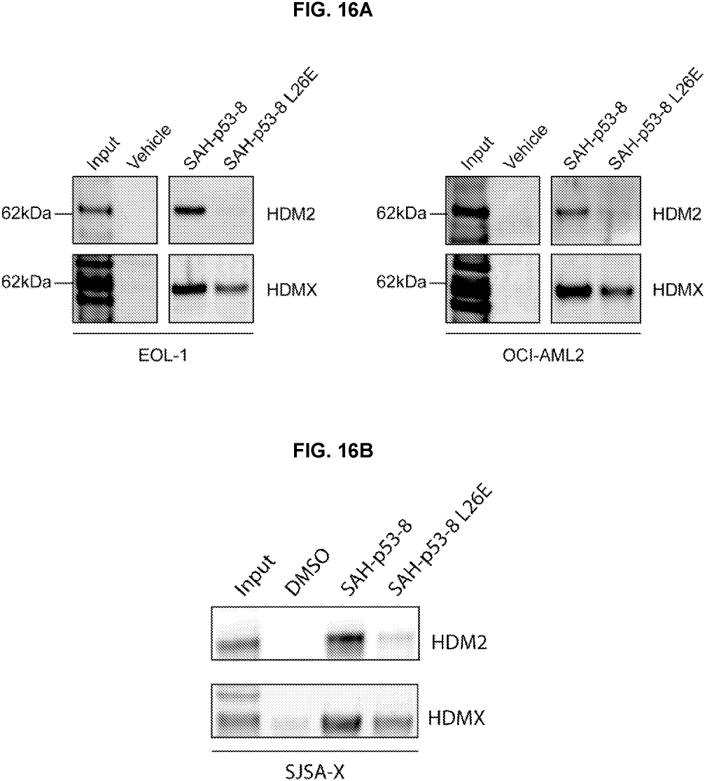

In order to determine if the insights gleaned from the biochemical and structural analyses (Examples 1 and 2, above) informed the development of a stapled p53 peptide with high fidelity selectivity for native HDMX from cells, binding assays were performed using cellular lysates from both hematologic (FIG. 16A) and solid (FIG. 16B) cancers. In each case, SAH-p53-8 effectively engaged both HDM2 and HDMX in EOL-1, OCI-AML2, and SJSA-X cancer cells, but upon L26E mutagenesis, HDMX binding activity was preserved and HDM2 binding activity was markedly impaired or abrogated (FIG. 16). These data demonstrate that L26E mutagenesis converts a dual-targeting HDM2/HDMX inhibitor into a selective HDMX inhibitor in the context of the native proteins from cancer cell lysates.

### Example 4: Development of a Selective HDMX Inhibitor Capable of Inducing Cancer Cell Cytotoxicity

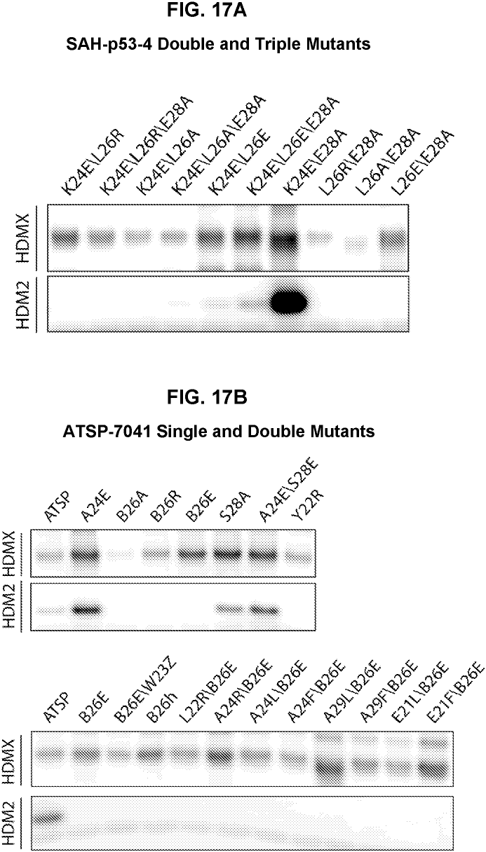

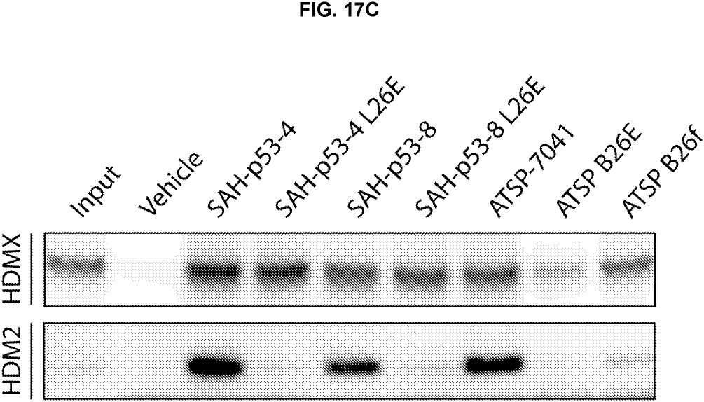

A major challenge in translating a biochemically-active stapled peptide into a cell penetrant agent capable of on-mechanism anti-cancer activity involves optimizing the compound for cellular uptake, pinosomal release, and avoidance of non-specific membrane lysis (Bird et al. Nat Chem Biol, 2016), followed by a series of requisite follow-on steps to optimize pharmacokinetics and pharmacodynamics in vivo. Because these parameters are not reliably predictable for the variety of stapled peptide templates and remain an active area of investigation, substantial compound iteration and testing is required. Indeed, modifications that maximize uptake and avoid membrane lysis may ultimately alter target specificity, requiring a cycle of iterative synthesis and testing to arrive at a lead compound with the desired litany of properties. Thus, iterative mutagenesis was performed involving single, double, and even triple amino acid substitutions within the SAH-p53 and ATSP-7041 template stapled p53 peptides in an effort to identify compounds that enhanced HDMX binding activity, retained HDMX binding selectivity, and optimized cellular uptake, and ultimately cancer cell cytotoxicity. A series of double and triple mutants of SAH-p53-4 retained HDMX binding selectivity, whereas some combinations unexpectedly restored dual HDM2/HDMX interaction, as evidenced in particular for the K24E/E28A mutant that confers especially robust HDM2 interaction along with HDMX binding, as assessed by competitive streptavidin pull-down of biotinylated peptides incubated with a 1:1 mixture of recombinant HDM2 and HDMX proteins, followed by HDM2 and HDMX western analyses (FIG. 17A). In the context of ATSP-7041, select single and double mutants of ATSP-7041 retained and even enhanced binding specificity toward HDMX (e.g. A24R/B26E, A29L/B26E, E21F/B26E), whereas other mutants restored dual HDM2/HDMX binding selectivity (e.g. A24E, S28A, A24E/S28E) (FIG. 17B).

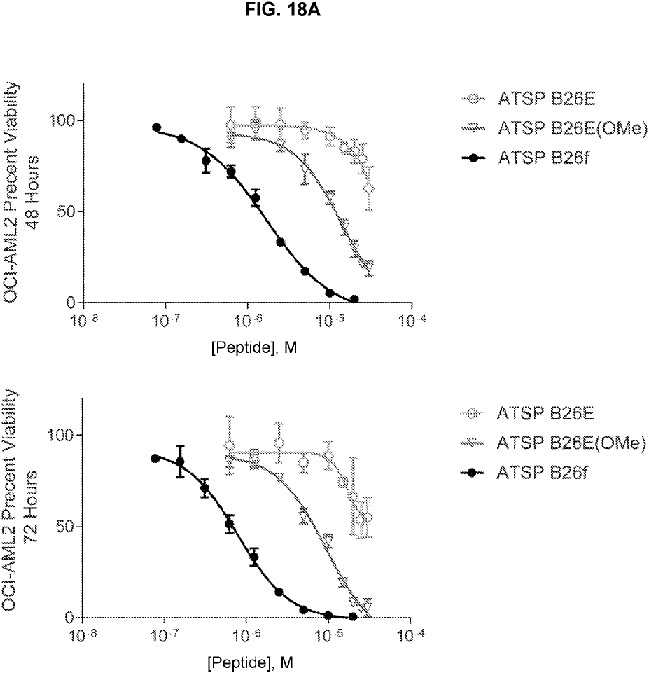

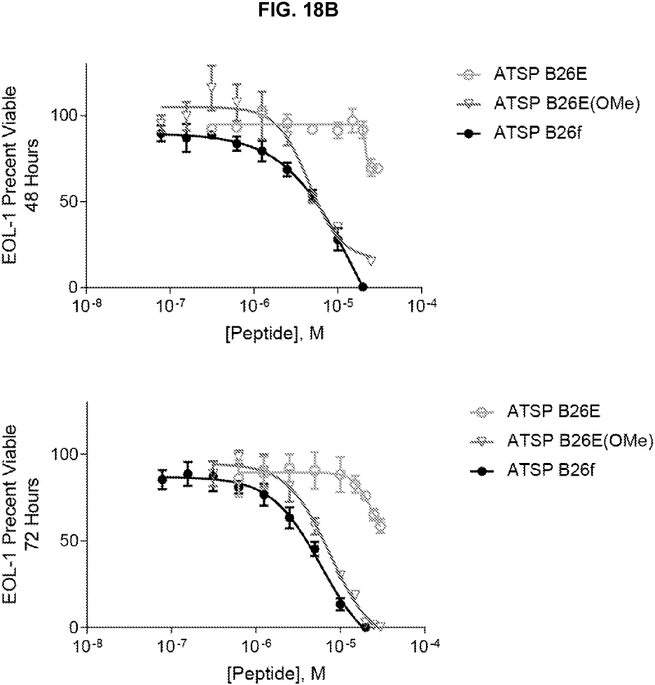

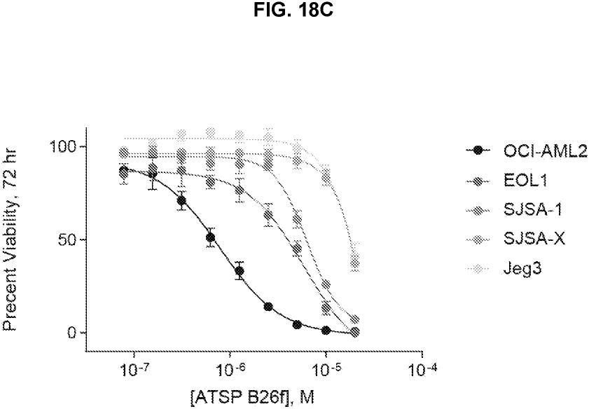

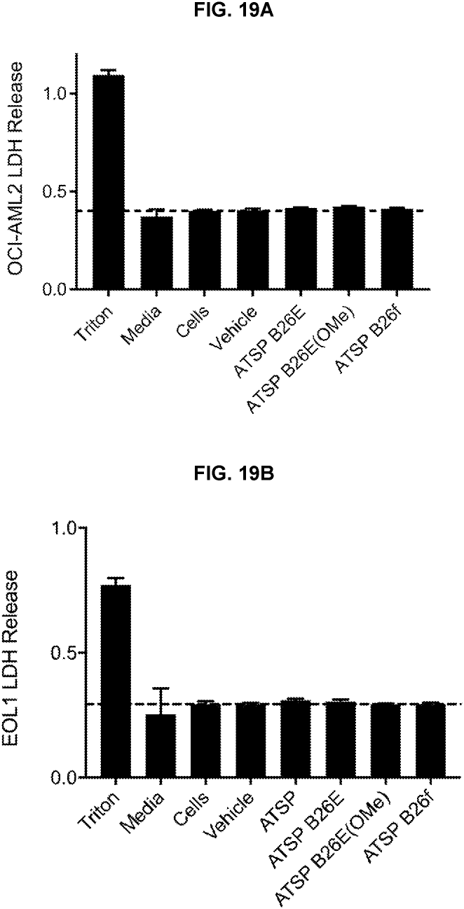

Because ATSP-7041 already has desirable cell penetrant properties, with effective in vivo anti-cancer activity in mouse models, converting this agent into an HDMX-selective agent was pursued. However, B26E mutagenesis - like L26E mutagenesis in the context of SAH-p53-4 and SAH-p53-8 - replaces a hydrophobic residue with a negatively charged residue, which mitigates cell uptake, resulting in decreased cell penetrance and cytotoxicity. Therefore, it was sought to optimize this position in SAH-p53-8 and ATSP-7041, for example, while maintaining HDMX-selectivity using a series of approaches including converting glutamate to its ester (OMe) and substituting glutamate with a 5-fluoronorvaline moiety (f). Indeed, the latter substitution in ATSP-7041 B26E enhanced HDMX binding activity compared to the parent compound while preserving HDMX-selectivity (FIG. 17C). Upon treatment of OCI-AML2 and EOL-1 cancer cells with the series of ATSP-7041 B26E, B26E(OMe), and B26f compounds, dose-responsive cancer cell cytotoxicity was observed, both at 48 and 72 hours of treatment, with serial improvement in potency based on the respective OMe and f design interventions (FIG. 18A and FIG. 18B). Cancer cell cytotoxicity was observed in both hematologic malignancies (OCI-AML2, EOL-1) and solid tumors (SJSA-1, SJSA-X, JEG-3), as demonstrated for ATSP B26f (FIG. 18C). Importantly, none of these compounds triggered non-specific membrane lysis (FIG. 19). Thus, this example demonstrates that an HDMX-selective stapled p53 peptide can be optimized to induce dose- and time-dependent cancer cell killing using full-serum media and without non-specific membrane lysis, highlighting the translational potential of the HDMX-selectivity determinant discovery described herein.

### Materials and Methods Used in Examples 1-4

Peptide Synthesis: SAH-p53 and ATSP-7041 peptides and their mutants were synthesized, derivatized with either biotin, acetyl or FITC at the N-terminus, purified, and quantified by amino acid analysis as previously described in detail (Bird et al., Methods in Enzymol, 2008). Stapled peptides were synthesized by solid phase Fmoc chemistry using rink-amide resin. Elongation of the peptides was performed with hexafluorophosphate azabenzotriazole tetramethyl uranium (HATU) as the coupling reagent and diisopropylethylamine (DIEA). Two naturally-occurring amino acids at discrete i, i+7 positions were replaced with the R-octenyl alanine and S-pentenyl alanine non-natural amino acids at the indicated locations to enable stapling by olefin metathesis using the Grubb’s first-generation catalyst. Peptides were deprotected and cleaved from the resin by a solution of 95:2.5:2.5 (v/v) TFA/TIS/water and purified by C18 reverse phase HPLC in acetonitrile/water gradient supplemented with 0.1% TFA to >95% purity. The pure peptides were lyophilized and stored at -20° C. until use. The following non-natural amino acids were used: Fmoc-Glu(OMe)-OH, Fmoc-Met(O)-OH, Fmoc-Homoglu(OtBu)-OH, Fmoc-3-(2-naphthyl)-L-alanine, Fmoc-b-cyclobutyl-L-alanine and Fmoc-5-fluoronorvaline-OH.

Recombinant Protein Expression and Purification: Recombinant HDM2 (amino acids 17-125 of SEQ ID NO: 103) and recombinant full-length (FL) HDMX (amino acids 1-490 of SEQ ID NO: 104) were generated as previously described (Bernal et al., 2007, 2010). The amino acid sequence of humanized zebrafish HDMX protein (amino acids 15-106, L46V/V95L) is set forth in SEQ ID NO:233. Recombinant HDM2 (amino acids 17-125 of SEQ ID NO: 103) with an N-terminal hexahistidine tag (SEQ IDNO:236) and a thrombin cleavage site was cloned into pET28a vector, expressed in BL21(DE3) E. coli and purified by sequential Ni-affinity and size-exclusion chromatography (SEC). Protein expression was induced with 1 mM isopropylthio-β-galactosidase (IPTG; Gold Biotechnology) for 4 hours at 30° C. Bacterial pellets were resuspended in lysis buffer (500 mM NaCl, 20 mM Tris, pH 8, containing 2 protease inhibitor tablets [Roche]) and lysed by two passes through a microfluidizer (M-110L, Microfluidics) chilled to 4° C. Insoluble cellular debris was pelleted by centrifugation (20,000 rpm, 45 minutes, 4° C.). The supernatant was clarified over a Ni-NTA (Qiagen) column pre-equilibrated with lysis buffer. The column was then sequentially washed with lysis buffer, wash buffer (300 mM NaCl, 20 mM Tris, pH 8) and wash buffer, and eluted with a gradient of imidazole ranging from 5 to 300 mM. The fractions containing the eluted protein were concentrated and purified on a Superdex S-75 (GE Healthcare) gel filtration system equilibrated with FPLC buffer (300 mM NaCl, 20 mM Hepes, pH 7.2). Protein purity and identity was confirmed by Coomassie staining and western blot analysis.

Recombinant FL HDMX with N-terminal thioredoxin, S, and hexahistidine tags (SEQ ID NO:236), and an engineered TEV protease cleavage site was cloned into pET32-LIC vector, and purified as described above for HDM2 except for the following modifications: fractions containing the eluted protein from the Ni-NTA column were buffer exchanged into FPLC buffer (as above) containing 5% glycerol and 50 mM arginine using a HiPrep 26/10 desalting column (GE Healthcare), concentrated, and treated overnight with recombinant AcTEV™ protease (ThermoFisher) at 4° C. The next morning cleaved protein was purified on a Superdex 200 gel filtration system as described above. Protein purity and identity was confirmed by Coomassie staining and western blot analysis.

Recombinant FL HDMX mutants were generated by PCR-based site directed mutagenesis (Q5 Site-Directed Mutagenesis Kit, NEB) using the following primers: HDMX Y99F Q5 mutagenesis, forward primer AGCCCTCTCTTTGATATGCTAAG (SEQ ID NO:229), reverse primer TGGGTCTTTCACGGAGAAG (SEQ ID NO:230); HDMX Y99A Q5 mutagenesis forward primer AAGCCCTCTCGCGGATATGCTAAGAAAG (SEQ ID NO:231), reverse primer GGGTCTTTCACGGAGAAGCTCTGACG (SEQ ID NO:232). Amino acid sequence of humanized zebrafish HDMX protein (amino acids 15-106, L46V/V95L):

LPGEGTQVHPRAPLLQILKVAGAQEEVFTVKEVMHYLGQYIMMKQLYDKQ
RQHIVHCHDDPLGELLEVGSFSVKNPSPLYEMLKRNLVIL (SEQ ID NO:233)

The humanized zebrafish HDMX construct was cloned, generated and purified as previously described (Chang et al. Proc Natl Acad Sci, 2013). Briefly, zHDMX (aa 15-106, L46V/V95L double mutation) bearing an N-terminal His₆ tag (SEQ ID NO:236) and enterokinase recognition sequence (pET15b vector, Novagen) was expressed in BL21(DE3) E.coli (0.2 mM IPTG, 4 hours induction at 30° C.) and purified by sequential Ni-affinity chromatography and SEC. Bacterial pellets were resuspended in lysis buffer (20 mM Tris pH 8, 500 mM NaCl, 2 protease inhibitor tablets [Roche]) and lysed by two passes through a microfluidizer (M-110L, Microfluidics) that was chilled to 4° C. Insoluble cellular debris was pelleted by centrifugation (20,000 rpm, 45 min, 4° C.) and the supernatant clarified over a Ni-NTA (Qiagen) column pre-equilibrated with lysis buffer. The column was sequentially washed with lysis buffer and wash buffer (20 mM Tris pH 8, 300 mM NaCl), and then eluted with a gradient of imidazole ranging from 5 to 300 mM in wash buffer. The fractions containing the eluted protein were dialyzed, concentrated and purified on a Superdex S-75 (GE Healthcare) gel filtration system equilibrated with FPLC buffer (50 mM NaPO4 pH 8, 150 mM NaCl, 2 mM TCEP). Protein purity and identity were confirmed by Coomassie staining and western blot analysis. For X-ray crystallography (see below), the protein buffer was exchanged to 20 mM Tris pH 8.0, 50 mM NaCl, 2 mM DTT.

Streptavidin Pull-Down - Recombinant Proteins: Recombinant wild-type/full-length (FL) HDMX and HDM2 (amino acids 17-125 of SEQ ID NO: 103), were combined (1:1, 0.5 µM each in FPLC buffer) and treated with 0.5 µM biotinlyted peptides or vehicle (0.25% DMSO) for 1 hour at 4° C. The peptide-protein complex was then incubated with pre-blocked (3% BSA, 2 hours, 4° C.) and equilibrated high-capacity Streptavidin agarose beads (30 µl per pull-down) for 2 hours at 4° C. The beads were washed three times each with 1% NP-40/FPLC buffer, T-BST and PBS. Bound protein was eluted from the beads by 10 minutes of boiling in 2x LDS supplemented with DTT, and then subjected to electrophoresis (input 10%, eluates 10 µL, 4-12% or 10% Bis-Tris gel) and western blot analysis. For comparative HDMX protein pull-downs, wild-type, Y99A, and Y99F HDMX proteins were individually incubated with biotinylated peptides and streptavidin pull-downs performed as above.

Isothermal Titration Calorimetry: ITC measurements were performed at 25° C. with 20 µM purified protein (cell) and titrating with 130 µM acetylated peptide (syringe) using an Affinity ITC (TA Instruments, New Castle, DE) equipped with an autosampler. Proteins and ligands were prepared in 3% DMSO/FPLC buffer containing 20 mM Tris pH 8.0, 50 mM NaCl, 2 mM DTT for zHDMX and 20 mM HEPES pH 7.2, 300 mM NaCl for HDM2. The data were analyzed using the NanoAnalyze software package (TA instrument) employing a single binding site model. Thermodynamic parameters were calculated according to the equation, ΔG = ΔH - TΔS = -RTlnKB, where ΔG, ΔH and ΔS are the changes in free energy, enthalpy and entropy of binding, respectively.

Fluorescence Polarization Binding Assays: Direct FP assays were performed as previously described (Bernal et al., 2007). Briefly, FITC peptides (25 nM) were incubated for 15 min with zHDMX or HDM2 (0.48 nM-2 µM) in binding assay buffer (0.01% Triton, 50 mM Tris pH 8, 140 mM NaCl), and then FP measured (485 nm excitation, 525 nm emission) on a SpectraMax M5 microplate reader (Molecular Devices). The Kd values were calculated by nonlinear regression analysis using Prism software (GraphPad).

Streptavidin Pull-Down - Cell Lysates: Cultured cells were trypsinized, washed once with cold PBS, and lysed on ice for 30 minutes in lysis buffer (0.5% NP-40, 150 mM NaCl, 50 mM Tris pH 7.4). Protein concentration of the isolated supernatant was determined by Bradford reagent. Lysate samples (2 mg) were incubated with vehicle (0.6% DMSO) or biotinylated peptides (15 nmole) overnight in lysis buffer at 4° C. Biotin capture was accomplished by incubating the mixture with equilibrated streptavidin agarose beads (50 µl per pull-down) for 2 hours at 4° C., followed by washing the pelleted beads with lysis buffer, TBS-T and PBS (3 times each). Bead-bound proteins were eluted by addition of 50 µl 2x LDS supplemented with DTT and boiling for 10 minutes. Eluted proteins were then subjected to electrophoresis and western blotting using HDM2 (IF2, EMD Millipore, 1:200) and MDM4 (A300-287A; Bethyl Laboratories, 1:1000) antibodies.

X-ray Crystallography: For co-crystallization with SAH-p53 and ATSP-7041 peptides, 7.8 mg/ml (652 µM) humanized zebrafish HDMX (4.8 mg/mL) was preincubated with 2x peptide (100 mM DMSO stock). An equal volume (100 nL) of HDMX was mixed with reservoir solution (50% PEG3350, 1 M sodium citrate pH = 6), and crystals were prepared in hanging drops at 12° C. Harvested crystals were flash-frozen in liquid nitrogen. Diffraction data from HDMX crystals were collected at beamline 24ID-C of the NE-CAT at the Advanced Photon Source (Argonne National Laboratory), and data sets were integrated and scaled using XIA2 package (Evans, 2006; Kabsch, 2010; Winter, 2010). The structure of the HDMX/peptide complex was solved by molecular replacement using the program Phaser (McCoy et al., 2007) and the search model PDB entry 4N5T. Iterative manual model building and refinement using Phenix (Adams et al., 2010) and Coot (Emsley and Cowtan, 2004) led to models with excellent statistics, including maximum diffraction of 1.62 Å (PDB ID 6V4E; SAH-p53-8/zHDMX), 1.25 Å (PDB ID 6V4G, SAH-p53-8 L26E/zHDMX), 1.53 Å (PDB ID 6V4H; SAH-p53-4/zHDMX), and 1.35 Å (PDB ID 6V4F; SAH-p53-4/zHDMX L26E)

Cell Viability Assays: OCI-AML2 cells were plated in 96-well opaque plates (1 × 10⁴ cells/well) in OptiMEM media containing 10% FBS and allowed to acclimate for 3 hours. The cells were then treated with the indicated concentrations of peptides or vehicle control (0.2% DMSO) in triplicate. Peptide stocks were diluted into water to achieve 10X working stocks, which were then diluted 10-fold into the treatment wells. Cell viability was measured by CellTiter-Glo assay (Promega) at the indicated time points, performed according to the manufacturer’s instructions, and percent viability calculated based on untreated control cells.

LDHRelease Assay: OCI-AML2 cells (1 × 10⁴ cells/well) were plated in 96-well plates and treated with the indicated peptides or vehicle (0.2% DMSO). After 1.5 hours incubation at 37° C., 100 µl of cell medium was transferred to a 96-well clear plate and incubated with 100 µl of LDH reagent (Roche) for 25 minutes while shaking, and absorbance at 490 nm measured on a microplate reader (SpectraMax M5 Microplate Reader, Molecular Devices).

What is claimed is:

1. A structurally-stabilized peptide that preferentially binds to HDMX over HDM2, wherein the structurally-stabilized peptide comprises the amino acid sequence LTFEEYWAQBTSAA (SEQ ID NO:101), with 3 to 10 amino acid substitutions relative to the amino acid sequence of SEQ ID NO: 101, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with a charged amino acid, alanine, or a hydrophilic amino acid, and optionally wherein the 3 to 10 amino acid substitutions comprises (i) substitution of the amino acid corresponding to F19 of SEQ ID NO: 102 with a charged amino acid or alanine, or (ii) substitution of the amino acid corresponding to W23 of SEQ ID NO: 102 with a charged amino acid, alanine, or an aromatic amino acid, wherein the structurally-stabilized peptide is stapled or stitched.

2. A structurally-stabilized peptide that preferentially binds to HDMX over HDM2, wherein the structurally -stabilized peptide comprises the amino acid sequence LSQETFSDLWKLLPEN (SEQ ID NO:99), with 3 to 10 amino acid substitutions relative to the amino acid sequence of SEQ ID NO:99, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with a charged amino acid, alanine, or a hydrophilic amino acid, and optionally wherein the 3 to 10 amino acid substitutions comprises: (i) substitution of the amino acid corresponding to F19 of SEQ ID NO: 102 with a charged amino acid or alanine, or (ii) substitution of the amino acid corresponding to W23 of SEQ ID NO: 102 with a charged amino acid, alanine, or an aromatic amino acid, wherein the structurally-stabilized peptide is stapled or stitched.

3. A structurally-stabilized peptide that preferentially binds to HDMX over HDM2, wherein the structurally-stabilized peptide comprises the amino acid sequence QSQQTFSNLWRLLPQN (SEQ ID NO:100), with 3 to 10 amino acid substitutions relative to the amino acid sequence of SEQ ID NO: 100, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with a charged amino acid, alanine, or a hydrophilic amino acid, and optionally wherein the 3 to 10 amino acid substitutions comprises: (i) substitution of the amino acid corresponding to F19 of SEQ ID NO: 102 with a charged amino acid or alanine, or (ii) substitution of the amino acid corresponding to W23 of SEQ ID NO: 102 with a charged amino acid, alanine, or an aromatic amino acid, wherein the structurally-stabilized peptide is stapled or stitched.

4. The structurally-stabilized peptide of any one of claims 1 to 3, wherein one of the 3 to 10 amino acid substitutions is a substitution of the amino acid corresponding to L26 of SEQ ID NO: 102 with a charged amino acid or alanine.

5. A structurally-stabilized peptide that preferentially binds to HDMX over HDM2, wherein the structurally-stabilized peptide comprises:
(a) the amino acid sequence LTF8EYWAQBXSAA, wherein 8 is a first stapling amino acid, X is a second stapling amino acid, and B is cyclobutyl alanine (SEQ ID NO:55), with 1 to 10 amino acid substitutions relative to the amino acid sequence of SEQ ID NO:55, wherein the 1 to 10 amino acid substitutions are not at positions 4 or 11 of SEQ ID NO:55, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid;
(b) the amino acid sequence LSQETF8DLWKLLXEN, wherein 8 is a first stapling amino acid and X is a second stapling amino acid (SEQ ID NO: 1), with 1 to 10 amino acid substitutions relative to the amino acid sequence of SEQ ID NO:1, wherein the 1 to 10 amino acid substitutions are not at positions 7 or 14 of SEQ ID NO: 1, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid; or
(c) the amino acid sequence QSQQTF8NLWRLLXQN, wherein 8 is a first stapling amino acid and X is a second stapling amino acid (SEQ ID NO:95), with 1 to 10 amino acid substitutions relative to the amino acid sequence of SEQ ID NO:95, wherein the 1 to 10 amino acid substitutions are not at positions 7 or 14 of SEQ ID NO:95, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid.

6. The structurally-stabilized peptide of claim 5, wherein the structurally-stabilized peptide preferentially binds to HDMX with at least 1.5-fold higher, at least 2-fold higher, at least 2.5-fold higher, at least 3-fold higher, at least 4-fold higher, at least 5-fold higher, at least 6-fold higher, at least 7-fold higher, at least 8-fold higher, at least 9-fold higher, at least 10-fold higher, at least 15-fold higher, or at least 20-fold higher binding affinity than a binding affinity for HDM2.

7. The structurally-stabilized peptide of claim 5 or 6, wherein the structurally-stabilized peptide comprises:
(a) the amino acid sequence LTF8EYWAQBXSAA, wherein 8 is a first stapling amino acid, X is a second stapling amino acid, and B is cyclobutyl alanine (SEQ ID NO:55), with 1 to 10 amino acid substitutions relative to the amino acid sequence of SEQ ID NO:55, wherein the 1 to 10 amino acid substitutions are not at positions 4 or 11 of SEQ ID NO:55, wherein at least one of the amino acid substitutions is at the amino acid position corresponding to E17, F19, L22, W23, K24, L26, or N29, or combinations thereof, of the amino acid sequence of SEQ ID NO: 102, and wherein a sidechain of the first stapling amino acid is cross-linked to a sidechain of the second stapling amino acid.

8. The structurally-stabilized peptide of claim 5 or 6, wherein the structurally-stabilized peptide comprises:
(b) the amino acid sequence LSQETF8DLWKLLXEN, wherein 8 is a first stapling amino acid and X is a second stapling amino acid (SEQ ID NO: 1), with 1 to 10 amino acid substitutions relative to the amino acid sequence of SEQ ID NO:1, wherein the 1 to 10 amino acid substitutions are not at positions 7 or 14 of SEQ ID NO: 1, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid.

9. The structurally-stabilized peptide of claim 5 or 6, wherein the structurally-stabilized peptide comprises:
(c) the amino acid sequence QSQQTF8NLWRLLXQN, wherein 8 is a first stapling amino acid and X is a second stapling amino acid (SEQ ID NO:95), with 1 to 10 amino acid substitutions relative to the amino acid sequence of SEQ ID NO:95, wherein the 1 to 10 amino acid substitutions are not at positions 7 or 14 of SEQ ID NO:95, wherein at least one of the amino acid substitutions is at the amino acid position corresponding to Q16, E17, F19, D21, L22, W23, K24, L26, or N29, or combinations thereof, of the amino acid sequence of SEQ ID NO: 102, and wherein a sidechain of the first stapling amino acid is cross-linked to a sidechain of the second stapling amino acid.

10. The structurally-stabilized peptide of any one of claims 5 to 9, wherein:
the substitution at the amino acid position corresponding to E17 of the amino acid sequence of SEQ ID NO: 102 is with a negatively charged amino acid;
the substitution at the amino acid position corresponding to F19 of the amino acid sequence of SEQ ID NO: 102 is with a charged amino acid or alanine;
the substitution at the amino acid position corresponding to L22 of the amino acid sequence of SEQ ID NO: 102 is with a positively charged amino acid;
the substitution at the amino acid position corresponding to W23 of the amino acid sequence of SEQ ID NO: 102 is with a charged amino acid, alanine, or an aromatic amino acid;
the substitution at the amino acid position corresponding to K24 of the amino acid sequence of SEQ ID NO: 102 is with a charged amino acid or an aromatic amino acid;
the substitution at the amino acid position corresponding to L26 of the amino acid sequence of SEQ ID NO: 102 is with a charged amino acid, alanine, a hydrophobic amino acid less hydrophobic than alanine, or a hydrophilic amino acid; or
the substitution at the amino acid position corresponding to N29 of the amino acid sequence of SEQ ID NO:102 is with a positively charged amino acid or an aromatic amino acid.

11. The structurally-stabilized peptide of claim 7, wherein the at least one of the amino acid substitutions is at the amino acid position corresponding to F19, L22, W23, K24, L26, and N29, or combinations thereof of the amino acid sequence of SEQ ID NO:102, wherein the substitution at position L22 is with a positively charged amino acid, the substitution at position W23 is with an aromatic amino acid, the substitution at position K24 is with a hydrophobic or positively charged amino acid, the substitution at position L26 is with a charged amino acid or alanine, and the substitution at position N29 is with a hydrophobic amino acid.

12. The structurally-stabilized peptide of claim 11, wherein the substitution at position L22 is with arginine (R), the substitution at position W23 is with naphthalene, the substitution at position K24 is with arginine (R), leucine (L), or phenylalanine (F), the substitution at position L26 is with alanine (A), arginine (R), glutamate (E), homoglutamic acid (h), 5-fluoronorvaline (f), O-methylated glutamic acid (E(OMe)), glycine (G), histidine (H), lysine (K), asparagine (N), glutamine (Q), serine (S), threonine (T), valine (V), tryptophan (W), tyrosine (Y), proline (P), or cysteine (C), and the substitution at position N29 is with leucine (L) or phenylalanine (F).

13. The structurally-stabilized peptide of claim 7, wherein the 1 to 10 amino acid substitutions corresponding to the amino acid sequence of SEQ ID NO:102 comprise one or more of:
(i) L26A;
(ii) L26R;
(iii) L26E;
(iv) L26h, wherein h is homoglutamic acid;
(v) L26f, wherein f is 5-fluoronorvaline (f);
(vi) L26E(OMe), wherein E(OMe) is O-methylated glutamic acid;
(vii) L26G;
(viii) L26H;
(ix) L26K;
(x) L26N;
(xi) L26Q;
(xii) L26S;
(xiii) L26T;
(xiv) L26V;
(xv) L26W;
(xvi) L26Y;
(xvii) L26P;
(xviii) L26C;
(xix) L26D;
(xx) L22R;
(xxi) W23Z and L26E, wherein Z is naphthalene;
(xxii) L22R and L26E;
(xxiii) K24R and L26E;
(xxiv) K24L and L26E;
(xxv) K24F and L26E;
(xxvi) N29L and L26E;
(xxvii) N29F and L26E;
(xxviii) D21L and L26E;
(xxix) D21F and L26E; or
(xxx) L26Met(O), wherein Met(O) is methionine-sulfoxide.

14. The structurally-stabilized peptide of any one of claims 7 and 11 to 13, which is 14 to 50 amino acids in length.

15. The structurally-stabilized peptide of claim 8, wherein the at least one of the amino acid substitutions is at the amino acid position corresponding to F19, L22, W23, K24, L26, and N29, or combinations thereof, wherein the substitution at position F19 is with a charged amino acid or alanine, the substitution at position L22 is with a positively charged amino acid, the substitution at position W23 is with a charged amino acid or alanine, the substitution at position K24 is with a negatively charged amino acid, the substitution at position L26 is with a charged amino acid or alanine, and the substitution at position N29 is with a positively charged amino acid.

16. The structurally-stabilized peptide of claim 15, wherein the substitution at position F19 is with alanine (A), arginine (R), or glutamate (E), the substitution at position L22 is with arginine (R), the substitution at position W23 is with alanine (A), arginine (R), or glutamate (E), the substitution at position K24 is with glutamate (E), the substitution at position L26 is with alanine (A), arginine (R), or glutamate (E), and the substitution at position N29 is with arginine (R).

17. The structurally-stabilized peptide of claim 8, wherein the 1-8 amino acid substitutions comprise one or more of:
(i) F19A;
(ii) F19R;
(iii) F19E;
(iv) L22R;
(v) W23A;
(vi) W23R;
(vii) W23E;
(viii) K24E;
(ix) L26A;
(x) L26R;
(xi) L26E;
(xii) N29R;
(xiii) K24E and L26R;
(xiv) K24E, L26R, and E28A;
(xv) K24E and L26A;
(xvi) K24E and L26E;
(xvii) K24E, L26E, and E28A;
(xviii) K24E, L26A and E28A;
(xix) L26R and E28A;
(xx) L26A and E28A; or
(xxi) L26E and E28A.

18. The structurally-stabilized peptide of claim 9, wherein the at least one of the amino acid substitutions is at the amino acid position corresponding to E17 and L26, or combinations thereof, wherein the substitution at position E17 is with a negatively charged amino acid and the substitution at position L26 is with a charged amino acid or alanine.

19. The structurally-stabilized peptide of claim 18, wherein the substitution at position E17 is with O-methylated glutamate (E(OMe)) and substitution at position L26 is with alanine (A), arginine (R), glutamate (E), O-methylated glutamate (E(OMe)), or Met(O) (wherein Met(O) is methionine-sulfoxide).

20. The structurally-stabilized peptide of claim 9, wherein the 1 to 10 amino acid substitutions comprise:
(i) L26E;
(ii) L26E(OMe); or
(iii) E17E(OMe) and L26E(OMe).

21. The structurally-stabilized peptide of any one of claims 8, 9, and 15 to 20, which is 16 to 50 amino acids in length.

22. The structurally-stabilized peptide of any one of claims 5 to 21, wherein 8 is (R)-α-(7′-octenyl)alanine and X is (S)-α-(4′-pentenyl)alanine, or wherein 8 is (R)-α-(4′-pentenyl)alanine and X is (S)-α-(7′-octenyl)alanine.

23. A structurally-stabilized peptide comprising an amino acid sequence set forth in any one of SEQ ID NOs:80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), wherein a sidechain of the first stapling amino acid is cross-linked to a sidechain of the second stapling amino acid.

24. The structurally-stabilized peptide of claim 23, which consists of the amino acid sequence set forth in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), wherein a sidechain of the first stapling amino acid is cross-linked to a sidechain of the second stapling amino acid.

25. The structurally-stabilized peptide of claim 23 or 24, wherein in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), 8 is (R)-α-(7′-octenyl)alanine and X is (S)-α-(4′-pentenyl)alanine, or 8 is (R)-α-(4′-pentenyl)alanine and X is (S)-α-(7′-octenyl)alanine.

26. A structurally-stabilized peptide comprising an amino acid sequence set forth in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), comprising 1 to 4 amino acid substitutions, wherein the structurally-stabilized peptide is stapled, and wherein the peptide selectively binds to HDMX.

27. The structurally-stabilized peptide of claim 26, wherein in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), 8 is (R)-α-(7′-octenyl)alanine and X is (S)-α-(4′-pentenyl)alanine or 8 is (R)-α-(4′-pentenyl)alanine and X is (S)-α-(7′-octenyl)alanine.

28. The structurally-stabilized peptide of any one of claims 23 and 25 to 27, comprising 16 to 50 amino acids.

29. A structurally-stabilized peptide that preferentially binds to HDMX over HDM2, wherein the structurally-stabilized peptide comprises the amino acid sequence LTFEEYWAQBTSAA (SEQ ID NO:101), with 3 to 10 amino acid substitutions relative to the amino acid sequence of SEQ ID NO: 101, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with any amino acid other than phenylalanine, isoleucine, norleucine, and leucine, and optionally wherein the 3 to 10 amino acid substitutions comprises (i) substitution of the amino acid corresponding to F19 of SEQ ID NO:102 with a charged amino acid or alanine, or (ii) substitution of the amino acid corresponding to W23 of SEQ ID NO: 102 with a charged amino acid, alanine, or an aromatic amino acid, wherein the structurally-stabilized peptide is stapled or stitched.

30. A structurally-stabilized peptide that preferentially binds to HDMX over HDM2, wherein the structurally -stabilized peptide comprises the amino acid sequence LSQETFSDLWKLLPEN (SEQ ID NO:99), with 3 to 10 amino acid substitutions relative to the amino acid sequence of SEQ ID NO:99, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with any amino acid other than phenylalanine, isoleucine, norleucine, and leucine, and optionally wherein the 3 to 10 amino acid substitutions comprises: (i) substitution of the amino acid corresponding to F19 of SEQ ID NO: 102 with a charged amino acid or alanine, or (ii) substitution of the amino acid corresponding to W23 of SEQ ID NO: 102 with a charged amino acid, alanine, or an aromatic amino acid, wherein the structurally-stabilized peptide is stapled or stitched.

31. A structurally-stabilized peptide that preferentially binds to HDMX over HDM2, wherein the structurally-stabilized peptide comprises the amino acid sequence QSQQTFSNLWRLLPQN (SEQ ID NO:100), with 3 to 10 amino acid substitutions relative to the amino acid sequence of SEQ ID NO: 100, wherein at least two of the 3 to 10 amino acid substitutions are substitutions with stapling amino acids, wherein one of the 3 to 10 amino acid substitutions is a substitution of the amino acid corresponding to L26 of SEQ ID NO: 102 with any amino acid other than phenylalanine, isoleucine, norleucine, and leucine, and optionally wherein the 3 to 10 amino acid substitutions comprises: (i) substitution of the amino acid corresponding to F19 of SEQ ID NO: 102 with a charged amino acid or alanine, or (ii) substitution of the amino acid corresponding to W23 of SEQ ID NO: 102 with a charged amino acid, alanine, or an aromatic amino acid, wherein the structurally-stabilized peptide is stapled or stitched.

32. A peptide comprising the amino acid sequence set forth in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98 or (SEQ ID NO:84)-napthalene-(SEQ ID NO:238).

33. A pharmaceutical composition comprising: (i) the structurally-stabilized peptide of any one of claims 1 to 28; and (ii) a pharmaceutically acceptable carrier.

34. A pharmaceutical composition comprising: (i) the structurally-stabilized peptide of any one of claims 29 to 31; and (ii) a pharmaceutically acceptable carrier.

35. A method of making a structurally-stabilized peptide, the method comprising:
(a) providing a peptide having the sequence set forth in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98 or (SEQ ID NO:84)-napthalene-(SEQ ID NO:238), and (b) cross-linking the peptide.

36. The method of claim 35, wherein:
(a) in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or or (SEQ ID NO:84)-napthalene-(SEQ ID NO:238), respectively, 8 is (R)-α-(7′-octenyl)alanine, and wherein X in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), respectively, is (S)-α-(4′-pentenyl)alanine; or
(b) in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238), respectively, 8 is (R)-α-(4′-pentenyl)alanine, and X in any one of SEQ ID NOs: 80, 86, 5, 7, 10, 13, 21, 23, 24, 27, 29, 34, 37, 38, 40, 42-47, 49-51, 57-59, 61, 62, 64, 67-76, 78, 87-94, and 96-98, or (SEQ ID NO:84)-napthalene-(SEQ ID NO:238) is (S)-α-(7′-octenyl)alanine.

37. A method of treating cancer in a human subject in need thereof, comprising administering to the subject a therapeutically effective amount of the structurally-stabilized peptide of any one of claims 1 to 28.

38. The method of claim 33, wherein the cancer is an HDMX-overexpressing or HDMX-dependent cancer.

39. The method of claim 33 or 34, wherein the cancer is a hematologic cancer.

40. The method of claim 33 or 34, wherein the cancer is a solid cancer.

41. The method of claim 33 or 34, wherein the cancer is an eosinophilic leukemia.

42. The method of claim 33 or 34, wherein the cancer is acute myeloid leukemia.

43. The method of claim 33 or 34, wherein the cancer is an osteosarcoma.

44. The method of claim 33, wherein the cancer is a pediatric cancer.

45. A method of treating cancer in a human subject in need thereof, comprising administering to the subject a therapeutically effective amount of the structurally-stabilized peptide of any one of claims 29 to 31.

46. The method of claim 45, wherein the cancer is an HDMX-overexpressing or HDMX-dependent cancer.

47. The method of claim 45 or 46, wherein the cancer is a hematologic cancer, a soldi cancer, an eosinophilic leukemia, acute myeloid leukemia, an osteosarcoma, or a pediatric cancer.

48. A structurally-stabilized peptide of any one of claims 1 to 28, for use in tailoring a more selective and non-toxic treatment for a patient identified to have an HDMX-dependent cancer.

49. A structurally-stabilized peptide of any one of claims 29 to 31, for use in tailoring a more selective and non-toxic treatment for a patient identified to have an HDMX-dependent cancer.

50. A method of selecting a treatment for a cancer in a human subject, the method comprising:
obtaining cancer cells from the subject;
determining that the cancer cells express or are dependent on HDMX; and
administering to the subject a therapeutically effective amount of the structurally-stabilized peptide of any one of claims 1 to 28.

51. The method of claim 50, further comprising determining that the structurally-stabilized peptide of any one of claims 1 to 28 is cytotoxic to the cancer cells obtained from the human subject in an ex vivo assay.

52. A method of selecting a treatment for a cancer in a human subject, the method comprising:
obtaining cancer cells from the subject;
determining that the cancer cells express or are dependent on HDMX; and
administering to the subject a therapeutically effective amount of the structurally-stabilized peptide of any one of claims 29 to 31.

53. A method of selecting a treatment for a cancer in a human subject, the method comprising:
obtaining cancer cells from the subject;
separating the cancer cells into a first sample and a second sample;
contacting the cancer cells of the first sample with a HDMX-specific inhibitor and the cancer cells of the second sample with a HDM2/HDMX dual inhibitor;
determining that the cancer cells of the first sample are killed substantially the same or better than the cancer cells of the second sample; and
selecting the HDMX-specific inhibitor for treatment of the cancer.

54. A structurally stabilized peptide comprising an internally cross-linked polypeptide comprising the amino acid sequence Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆- (SEQ ID NO: 147), wherein:
Xaa₁ is Leu or a conservative amino acid substitution thereof, or Gln or a conservative amino acid substitution thereof, or is missing;
Xaa₂ is Ser or a conservative amino acid substitution thereof, or is missing;
Xaa₃ is Gln or a conservative amino acid substitution thereof, or is missing;
Xaa₄ is Glu or a conservative amino acid substitution thereof,, Leu, Ala, Gln or a conservative amino acid substitution thereof,;
Xaas is Thr or a conservative amino acid substitution thereof, or Ala;
Xaa₆ is Phe or a conservative substitution thereof, Ala, Arg, Glu, or a naphthalene;
Xaa₇ is Ser or Glu;
Xaas is Asp or a conservative amino acid substitution thereof, Glu, Ala, or Asn or a conservative amino acid substitution thereof;
Xaa₉ is Leu or a conservative amino acid substitution thereof, Tyr, or Arg;
Xaa₁₀ is Trp or a conservative substitution thereof, Ala, Arg, or Glu;
Xaa₁₁ is Lys or a conservative amino acid substitution thereof, Ala, Arg or a conservative amino acid substitution thereof, or Glu;
Xaa₁₂ is Leu or a conservative amino acid substitution thereof, G Lys, or Ala;
Xaa₁₃ is a charged amino acid, a hydrophobic amino acid less hydrophobic than alanine, a hydrophilic amino acid, Ala, Arg, Glu, E(OMe), Met(O), homoglutamic acid, 5-fluoronorvaline, Gly, His, Lys, Asn, Gln, Ser, Thr, Val, Trp, Tyr, Pro, or Cys;
Xaa₁₄ is Pro or Thr;
Xaa₁₅ is Glu or a conservative amino acid substitution thereof, Ser, Gln or a conservative amino acid substitution thereof, or Ala;
Xaa₁₆ is Asn or a conservative amino acid substitution thereof or Ala, or is missing;
wherein the side chains of at least two amino acids of the amino acid sequence separated by three or six amino acids are replaced by an internal cross-link, and wherein the internally cross-linked polypeptide preferentially binds HDMX over HDM2.

55. The structurally-stabilized peptide of claim 54, wherein the internally cross-linked polypeptide is alpha helical, neutral to positively charged, cell permeable, and/or not ubiquitinylated.

56. The structurally-stabilized peptide of claim 54 or 55, wherein Xaa₇ and Xaa₁₄ are stapling amino acids.

57. The structurally-stabilized peptide of any one of claims 54-56, wherein Xaa₇ is (R)-α-(7′-octenyl)alanine and Xaa₁₄ is (S)-α-(4′-pentenyl)alanine, or wherein Xaa₇ is (R)-α-(4′-pentenyl)alanine and Xaa₁₄ is (S)-α-(7′-octenyl)alanine.

58. The structurally-stabilized peptide of any one of claims 54-57, wherein the peptide comprising the internal cross-link is 17 to 50 amino acids in length.

59. The structurally-stabilized peptide of claim 1, wherein the structurally-stabilized peptide comprises the amino acid sequence of any one of SEQ ID NO: 59, 80, or 86.

60. The structurally stabilized peptide of claim 3, wherein the structurally-stabilized peptide comprises the amino acid sequence of any one of SEQ ID NO: 96 or 97.

61. A compound comprising a structurally-stabilized peptide comprising a cross-linked amino acid sequence having the formula: 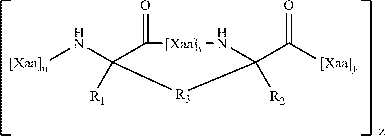
or a pharmaceutically acceptable salt thereof, wherein:
each R₁ and R₂ is independently H, alkyl, alkenyl, alkynyl, arylalkyl, cycloalkylalkyl, heteroarylalkyl, or heterocyclylalkyl, any of which is substituted or unsubstituted;
each R₃ is independently alkylene, alkenylene, or alkynylene, any of which is substituted or unsubstituted;
wherein the cross-linked amino acid sequence has the sequence set forth in SEQ ID NO: 100 or 101 with at least two amino acid substitutions, where the at least two amino acid substitutions include substitutions of two amino acids in SEQ ID NO: 100 or 101 with a stapling amino acid the side chains of which are cross-linked, wherein the structurally-stabilized peptide selectively binds HDMX over HDM2, and optionally wherein the amino acid sequence includes Phe19 and Trp23 of wild type p53.

62. The compound or the pharmaceutically acceptable salt thereof of claim 61, wherein the at least two amino acid substitutions are 3 to 10 amino acid substitutions, optionally 3 to 7, 3 to 6, 3 to 5 or 4 amino acid substitutions.

63. The compound or the pharmaceutically acceptable salt thereof of claim 61 or 62, wherein:
(a) [Xaa]_{w} is LTF, [Xaa]ₓ is EYWAQE (SEQ ID NO:194), and [Xaa]_{y} is SAA;
(b) [Xaa]_{w} is LTF, [Xaa]ₓ is EYWAQ# (SEQ ID NO:228), and [Xaa]_{y} is SAA;
(c) [Xaa]_{w} is LTF, [Xaa]ₓ is EYWAQf (SEQ ID NO:218), and [Xaa]_{y} is SAA;
(d) [Xaa]_{w} is QSQQTF (SEQ ID NO:225), [Xaa]ₓ is NLWRLE (SEQ ID NO:226), and [Xaa]_{y} is QN; or
(e) [Xaa]_{w} is QSQQTF (SEQ ID NO:225), [Xaa]ₓ is NLWRL# (SEQ ID NO:237), and [Xaa]_{y} is QN;
wherein # is E(OMe) and f is 5-fluoronorvaline.

64. The compound or the pharmaceutically acceptable salt thereof of any one of claims 61 to 63, wherein the pharmaceutically acceptable salt is acetate, sulfate, or chloride.

65. A structurally-stabilized peptide comprising a peptide set forth in any one of SEQ ID NOs.: 59, 80, 86, 96, or 97 with 1 to 8 amino acid substitutions, wherein the substitutions are not at locations of stapling amino acids in the peptide, and optionally wherein the peptide includes Phe19 and Trp23 of wild type p53.

66. The structurally-stabilized peptide of claim 65, having a length of 14-50 amino acids.

67. The structurally-stabilized peptide of claim 65 or 66, wherein 1 to 7, 1 to 6, 1 to 5, 1 to 4, 1 to 3, 1 to 2, or 1 amino acid substitutions are on the non-interacting face of the helix.

68. The structurally-stabilized peptide of any one of claim 65 to 67, wherein 1 to 5, 1 to 4, 1 to 3, 1 to 2, or 1 of the amino acids of the interacting face are substituted.

69. A method of treating a HDMX-expressing or dependent cancer in a human subject, the method comprising administering to the human subject a therapeutically effective amount of the compound or the pharmaceutically acceptable salt thereof of any one of claims 61 to 64, or the structurally-stabilized peptide of any one of claims 65 to 68.

70. A structurally-stabilized peptide that preferentially binds HDM2 over HDMX, wherein the structurally-stabilized peptide comprises the amino acid sequence set forth in SEQ ID NO:47 or a variant thereof, optionally wherein the variant of the amino acid sequence set forth in SEQ ID NO:47 comprises an amino acid sequence having 1 to 10 amino acid substitutions relative to the amino acid sequence set forth in SEQ ID NO:47.

71. A structurally-stabilized peptide that binds HDM2 and HDMX, wherein the structurally-stabilized peptide comprises the amino acid sequence set forth in any one of SEQ ID NOs: 2-4, 6, 9, 11, 12, 14-20, 22, 25, 26, 28, 30, 31-33, 35, 36, 39, 41, 47, 60, 63, 65, 66, 77, 79, and 81 or a variant thereof, optionally wherein the variant of the amino acid sequence set forth in any one of SEQ ID NOs: 2-4, 6, 9, 11, 12, 14-20, 22, 25, 26, 28, 30, 31-33, 35, 36, 39, 41, 47, 60, 63, 65, 66, 77, 79, and 81 comprises an amino acid sequence having 1 to 10 amino acid substitutions relative to the amino acid sequence set forth in any one of SEQ ID NOs: 2-4, 6, 9, 11, 12, 14-20, 22, 25, 26, 28, 30, 31-33, 35, 36, 39, 41, 47, 60, 63, 65, 66, 77, 79, and 81, respectively.

72. A pharmaceutical composition comprising: (i) the structurally-stabilized peptide of claim 70 or 71; and (ii) a pharmaceutically acceptable carrier.

73. A method of making a structurally-stabilized peptide, the method comprising:
(a) providing a peptide having the sequence set forth in any one of SEQ ID NOs: 2-4, 6, 9, 11, 12, 14-20, 22, 25, 26, 28, 30, 31-33, 35, 36, 39, 41, 47, 60, 63, 65, 66, 77, 79, and 81, or a variant thereof, and (b) cross-linking the peptide, optionally wherein the variant of the amino acid sequence set forth in any one of SEQ ID NOs: 2-4, 6, 9, 11, 12, 14-20, 22, 25, 26, 28, 30, 31-33, 35, 36, 39, 41, 47, 60, 63, 65, 66, 77, 79, and 81 comprises an amino acid sequence having 1 to 10 amino acid substitutions relative to the amino acid sequence set forth in any one of SEQ ID NOs: 2-4, 6, 9, 11, 12, 14-20, 22, 25, 26, 28, 30, 31-33, 35, 36, 39, 41, 47, 60, 63, 65, 66, 77, 79, and 81, respectively.

74. A method of treating cancer in a human subject in need thereof, comprising administering to the subject a therapeutically effective amount of the structurally-stabilized peptide of claim 70 or 71.

75. A pharmaceutical composition comprising (a) means for preferentially binding HDMX over HDM2; and (b) a pharmaceutically acceptable carrier.

76. A pharmaceutical composition comprising (a) means for preferentially binding HDM2 over HDMX; and (b) a pharmaceutically acceptable carrier.

77. A pharmaceutical composition comprising (a) means for selectively binding HDM2 and HDMX; and (b) a pharmaceutically acceptable carrier.


SEQUENCE LISTING (242 sequences)

SEQ ID NO: 1 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLLXEN

SEQ ID NO: 2 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
ASQETFXDLWKLLXEN

SEQ ID NO: 3 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LAQETFXDLWKLLXEN

SEQ ID NO: 4 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSAETFXDLWKLLXEN

SEQ ID NO: 5 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQATFXDLWKLLXEN

SEQ ID NO: 6 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQEAFXDLWKLLXEN

SEQ ID NO: 7 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETAXDLWKLLXEN

SEQ ID NO: 8 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXALWKLLXEN

SEQ ID NO: 9 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDAWKLLXEN

SEQ ID NO: 10 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLAKLLXEN

SEQ ID NO: 11 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWALLXEN

SEQ ID NO: 12 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKALXEN

SEQ ID NO: 13 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLAXEN

SEQ ID NO: 14 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLLXAN

SEQ ID NO: 15 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLLXEA

SEQ ID NO: 16 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
RSQETFXDLWKLLXEN

SEQ ID NO: 17 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LRQETFXDLWKLLXEN

SEQ ID NO: 18 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSRETFXDLWKLLXEN

SEQ ID NO: 19 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQRTFXDLWKLLXEN

SEQ ID NO: 20 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQERFXDLWKLLXEN

SEQ ID NO: 21 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETRXDLWKLLXEN

SEQ ID NO: 22 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXRLWKLLXEN

SEQ ID NO: 23 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDRWKLLXEN

SEQ ID NO: 24 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLRKLLXEN

SEQ ID NO: 25 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWRLLXEN

SEQ ID NO: 26 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKRLXEN

SEQ ID NO: 27 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLRXEN

SEQ ID NO: 28 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLLXRN

SEQ ID NO: 29 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLLXER

SEQ ID NO: 30 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
ESQETFXDLWKLLXEN

SEQ ID NO: 31 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LEQETFXDLWKLLXEN

SEQ ID NO: 32 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSEETFXDLWKLLXEN

SEQ ID NO: 33 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQEEFXDLWKLLXEN

SEQ ID NO: 34 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETEXDLWKLLXEN

SEQ ID NO: 35 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXELWKLLXEN

SEQ ID NO: 36 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDEWKLLXEN

SEQ ID NO: 37 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLEKLLXEN

SEQ ID NO: 38 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELLXEN

SEQ ID NO: 39 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKELXEN

SEQ ID NO: 40 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLEXEN

SEQ ID NO: 41 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLLXEE

SEQ ID NO: 42 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELRXEN

SEQ ID NO: 43 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELRXAN

SEQ ID NO: 44 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELAXEN

SEQ ID NO: 45 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELEXEN

SEQ ID NO: 46 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELEXAN

SEQ ID NO: 47 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELLXAN

SEQ ID NO: 48 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWELAXAN

SEQ ID NO: 49 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLRXAN

SEQ ID NO: 50 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLAXAN

SEQ ID NO: 51 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDLWKLEXAN

SEQ ID NO: 52 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDRWKLEXEN

SEQ ID NO: 53 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDRWKLRXEN

SEQ ID NO: 54 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
LSQETFXDRWKLAXEN

SEQ ID NO: 55 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWAQXXSAA

SEQ ID NO: 56 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQDXSAA

SEQ ID NO: 57 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQAXSAA

SEQ ID NO: 58 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQRXSAA

SEQ ID NO: 59 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQEXSAA

SEQ ID NO: 60 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQFXSAA

SEQ ID NO: 61 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQGXSAA

SEQ ID NO: 62 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQHXSAA

SEQ ID NO: 63 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQIXSAA

SEQ ID NO: 64 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQKXSAA

SEQ ID NO: 65 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQLXSAA

SEQ ID NO: 66 - protein, 14 aa, Artificial Sequence
  source: /note="Description of Artificial Sequence: Synthetic peptide"
  MOD_RES (4)..(4): stapling amino acid
  MOD_RES (10)..(10): norleucine
  MOD_RES (11)..(11): stapling amino acid
LTFXEYWAQXXSAA

SEQ ID NO: 67 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQNXSAA

SEQ ID NO: 68 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQQXSAA

SEQ ID NO: 69 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQSXSAA

SEQ ID NO: 70 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQTXSAA

SEQ ID NO: 71 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQVXSAA

SEQ ID NO: 72 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQWXSAA

SEQ ID NO: 73 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQYXSAA

SEQ ID NO: 74 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQPXSAA

SEQ ID NO: 75 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQCXSAA

SEQ ID NO: 76 - protein, 14 aa, Artificial Sequence
  source: /note="Description of Artificial Sequence: Synthetic peptide"
  MOD_RES (4)..(4): stapling amino acid
  MOD_RES (10)..(10): norleucine
  MOD_RES (11)..(11): stapling amino acid
LTFXERWAQXXSAA

SEQ ID NO: 77 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWEQXXSAA

SEQ ID NO: 78 - protein, 14 aa, Artificial Sequence
  source: /note="Description of Artificial Sequence: Synthetic peptide"
  MOD_RES (4)..(4): stapling amino acid
  MOD_RES (10)..(10): homoglutamic acid
  MOD_RES (11)..(11): stapling amino acid
LTFXEYWAQXXSAA

SEQ ID NO: 79 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWAQXXAAA

SEQ ID NO: 80 - protein, 14 aa, Artificial Sequence
  source: /note="Description of Artificial Sequence: Synthetic peptide"
  MOD_RES (4)..(4): stapling amino acid
  MOD_RES (10)..(10): E(OMe)
  MOD_RES (11)..(11): stapling amino acid
LTFXEYWAQXXSAA

SEQ ID NO: 81 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWEQXXEAA

SEQ ID NO: 82 - protein, 14 aa, Artificial Sequence
  source: /note="Description of Artificial Sequence: Synthetic peptide"
  MOD_RES (4)..(4): stapling amino acid
  MOD_RES (10)..(10): methionine-sulfoxide
  MOD_RES (11)..(11): stapling amino acid
LTFXEYWAQXXSAA

SEQ ID NO: 83 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXERWAQXXSAA

SEQ ID NO: 84 - protein, 6 aa, Artificial Sequence
  source: /note="Description of Artificial Sequence: Synthetic peptide"
  MOD_RES (4)..(4): stapling amino acid
LTFXEY

SEQ ID NO: 85 - protein, 14 aa, Artificial Sequence
  source: /note="Description of Artificial Sequence: Synthetic peptide"
  MOD_RES (4)..(4): stapling amino acid
  MOD_RES (5)..(5): E(OMe)
  MOD_RES (10)..(10): E(OMe)
  MOD_RES (11)..(11): stapling amino acid
LTFXXYWAQXXSAA

SEQ ID NO: 86 - protein, 14 aa, Artificial Sequence
  source: /note="Description of Artificial Sequence: Synthetic peptide"
  MOD_RES (4)..(4): stapling amino acid
  MOD_RES (10)..(10): 5-fluoronorvaline
  MOD_RES (11)..(11): stapling amino acid
LTFXEYWAQXXSAA

SEQ ID NO: 87 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWRQEXSAA

SEQ ID NO: 88 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWLQEXSAA

SEQ ID NO: 89 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQEXSLA

SEQ ID NO: 90 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXLYWAQEXSAA

SEQ ID NO: 91 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWFQEXSAA

SEQ ID NO: 92 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXEYWAQEXSFA

SEQ ID NO: 93 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXFYWAQEXSAA

SEQ ID NO: 94 - protein, 14 aa, Artificial Sequence
  source: /note="Description of Artificial Sequence: Synthetic peptide"
  MOD_RES (4)..(4): stapling amino acid
  MOD_RES (11)..(11): stapling amino acid
LTFXERWAQEXSAA

SEQ ID NO: 95 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
QSQQTFXNLWRLLXQN

SEQ ID NO: 96 - protein, 16 aa, Artificial Sequence
  source: /note="Description of Artificial Sequence: Synthetic peptide"
  MOD_RES (7)..(7): stapling amino acid
  MOD_RES (14)..(14): stapling amino acid
QSQQTFXNLWRLEXQN

SEQ ID NO: 97 - protein, 16 aa, Artificial Sequence
  source: /note="Description of Artificial Sequence: Synthetic peptide"
  MOD_RES (7)..(7): stapling amino acid
  MOD_RES (13)..(13): E(OMe)
  MOD_RES (14)..(14): stapling amino acid
QSQQTFXNLWRLXXQN

SEQ ID NO: 98 - protein, 16 aa, Artificial Sequence
  source: /note="Description of Artificial Sequence: Synthetic peptide"
  MOD_RES (4)..(4): E(OMe)
  MOD_RES (7)..(7): stapling amino acid
  MOD_RES (13)..(13): E(OMe)
  MOD_RES (14)..(14): stapling amino acid
QSQXTFXNLWRLXXQN

SEQ ID NO: 99 - protein, 16 aa, Unknown
  source: /note="Description of Unknown: p53 transactivation domain"
LSQETFSDLWKLLPEN

SEQ ID NO: 100 - protein, 16 aa, Artificial Sequence
  source: /note="Description of Artificial Sequence: Synthetic peptide"
QSQQTFSNLWRLLPQN

SEQ ID NO: 101 - protein, 14 aa, Artificial Sequence
  source: /note="Description of Artificial Sequence: Synthetic peptide"
  MOD_RES (10)..(10): cyclobutylalanine
LTFEEYWAQXTSAA

SEQ ID NO: 102 - protein, 393 aa, Homo sapiens [sequence omitted]

SEQ ID NO: 103 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 104 - protein, 490 aa, Homo sapiens
MTSFSTSAQCSTSDSACRISPGQINQVRPKLPLLKILHAAGAQGEMFTVKEVMHYLGQYIMVKQLYDQQEQHMVYCGGDLLGELLGRQSFSVKDPSPLYDMLRKNLVTLATATTDAAQTLALAQDHSMDIPSQDQLKQSAEESSTSRKRTTEDDIPTLPTSEHKCIHSREDEDLIENLAQDETSRLDLGFEEWDVAGLPWWFLGNLRSNYTPRSNGSTDLQTNQDVGTAIVSDTTDDLWFLNESVSEQLGVGIKVEAADTEQTSEEVGKVSDKKVIEVGKNDDLEDSKSLSDDTDVEVTSEDEWQCTECKKFNSPSKRYCFRCWALRKDWYSDCSKLTHSLSTSDITAIPEKENEGNDVPDCRRTISAPVVRPKDAYIKKENSKLFDPCNSVEFLDLAHSSESQETISSMGEQLDNLSEQRTDTENMEDCQNLLKPCSLCEKRPRDGNIIHGRTGHLVTCFHCARRLKKAGASCPICKKEIQLVIKVFIA

SEQ ID NO: 105 - protein, 16 aa, Artificial Sequence
  source: /note="Description of Artificial Sequence: Synthetic peptide"
LSEETFSDLWKLLPEN

SEQ ID NO: 106 - protein, 16 aa, Artificial Sequence
  source: /note="Description of Artificial Sequence: Synthetic peptide"
LSQATFSDLWKLLPEN

SEQ ID NO: 107 - protein, 16 aa, Artificial Sequence
  source: /note="Description of Artificial Sequence: Synthetic peptide"
  VARIANT (6)..(6): /replace="Arg" or "Glu"
  SITE (1)..(16): /note="Variant residues given in the sequence have no preference with respect to those in the annotations for variant positions"
LSQETASDLWKLLPEN

SEQ ID NO: 108 - protein, 16 aa, Artificial Sequence
  source: /note="Description of Artificial Sequence: Synthetic peptide"
LSQETFSALWKLLPEN

SEQ ID NO: 109 - protein, 16 aa, Artificial Sequence
  source: /note="Description of Artificial Sequence: Synthetic peptide"
LSQETFSDRWKLLPEN

SEQ ID NO: 110 - protein, 16 aa, Artificial Sequence
  source: /note="Description of Artificial Sequence: Synthetic peptide"
  VARIANT (10)..(10): /replace="Arg" or "Glu"
  SITE (1)..(16): /note="Variant residues given in the sequence have no preference with respect to those in the annotations for variant positions"
LSQETFSDLAKLLPEN

SEQ ID NO: 111 - protein, 16 aa, Artificial Sequence
  source: /note="Description of Artificial Sequence: Synthetic peptide"
LSQETFSDLWELLPEN

SEQ ID NO: 112 - protein, 16 aa, Artificial Sequence
  source: /note="Description of Artificial Sequence: Synthetic peptide"
  VARIANT (13)..(13): /replace="Arg" or "Glu"
  SITE (1)..(16): /note="Variant residues given in the sequence have no preference with respect to those in the annotations for variant positions"
LSQETFSDLWELAPEN

SEQ ID NO: 113 - protein, 16 aa, Artificial Sequence
  source: /note="Description of Artificial Sequence: Synthetic peptide"
  VARIANT (13)..(13): /replace="Arg" or "Glu"
  SITE (1)..(16): /note="Variant residues given in the sequence have no preference with respect to those in the annotations for variant positions"
LSQETFSDLWELAPAN

SEQ ID NO: 114 - protein, 16 aa, Artificial Sequence
  source: /note="Description of Artificial Sequence: Synthetic peptide"
  VARIANT (13)..(13): /replace="Arg" or "Glu"
  SITE (1)..(16): /note="Variant residues given in the sequence have no preference with respect to those in the annotations for variant positions"
LSQETFSDLWKLAPEN

SEQ ID NO: 115 - protein, 16 aa, Artificial Sequence
  source: /note="Description of Artificial Sequence: Synthetic peptide"
  VARIANT (13)..(13): /replace="Arg" or "Glu"
  SITE (1)..(16): /note="Variant residues given in the sequence have no preference with respect to those in the annotations for variant positions"
LSQETFSDLWKLAPAN

SEQ ID NO: 116 - protein, 16 aa, Artificial Sequence
  source: /note="Description of Artificial Sequence: Synthetic peptide"
LSQETFSDLWKLLPER

SEQ ID NO: 117 - protein, 14 aa, Artificial Sequence
  source: /note="Description of Artificial Sequence: Synthetic peptide"
  VARIANT (10)..(10): /replace="Ala" or "Arg" or "Glu" or "Gly" or "His" or "Lys" or "Asn" or "Gln" or "Ser" or "Thr" or "Val" or "Trp" or "Tyr" or "Pro" or "Cys" or "homoglutamic acid" or "E(OMe)" or "5-fluoronorvaline"
  SITE (1)..(14): /note="Variant residues given in the sequence have no preference with respect to those in the annotations for variant positions"
LTFEEYWAQDTSAA

SEQ ID NO: 118 - protein, 6 aa, Artificial Sequence
  source: /note="Description of Artificial Sequence: Synthetic peptide"
LTFEEY

SEQ ID NO: 119 - protein, 14 aa, Artificial Sequence
  source: /note="Description of Artificial Sequence: Synthetic peptide"
  VARIANT (8)..(8): /replace="Leu" or "Phe"
  SITE (1)..(14): /note="Variant residues given in the sequence have no preference with respect to those in the annotations for variant positions"
LTFEEYWRQETAAA

SEQ ID NO: 120 - protein, 14 aa, Artificial Sequence
  source: /note="Description of Artificial Sequence: Synthetic peptide"
  VARIANT (13)..(13): /replace="Phe"
  SITE (1)..(14): /note="Variant residues given in the sequence have no preference with respect to those in the annotations for variant positions"
LTFEEYWAQETSLA

SEQ ID NO: 121 - protein, 14 aa, Artificial Sequence
  source: /note="Description of Artificial Sequence: Synthetic peptide"
  VARIANT (5)..(5): /replace="Phe"
  SITE (1)..(14): /note="Variant residues given in the sequence have no preference with respect to those in the annotations for variant positions"
LTFELYWAQETSAA

SEQ ID NO: 122 - protein, 14 aa, Artificial Sequence
  source: /note="Description of Artificial Sequence: Synthetic peptide"
LTFEERWAQETSAA

SEQ ID NO: 123 - protein, 16 aa, Artificial Sequence
  source: /note="Description of Artificial Sequence: Synthetic peptide"
  VARIANT (13)..(13): /replace="E(OMe)"
  SITE (1)..(16): /note="Variant residues given in the sequence have no preference with respect to those in the annotations for variant positions"
QSQQTFSNLWRLEPQN

SEQ ID NO: 124 - protein, 17 aa, Artificial Sequence
  source: /note="Description of Artificial Sequence: Synthetic peptide"
  MOD_RES (4)..(4): E(OMe)
  MOD_RES (8)..(8): stapling amino acid
  MOD_RES (14)..(14): E(OMe)
QSQXTFSXNLWRLXPQN

SEQ ID NO: 125 - protein, 16 aa, Artificial Sequence
  source: /note="Description of Artificial Sequence: Synthetic peptide"
LSQETFSDLWELLPAN

SEQ ID NO: 126 - protein, 16 aa, Artificial Sequence
  source: /note="Description of Artificial Sequence: Synthetic peptide"
  VARIANT (1)..(1): /replace="Arg" or "Glu"
  SITE (1)..(16): /note="Variant residues given in the sequence have no preference with respect to those in the annotations for variant positions"
ASQETFSDLWKLLPEN

SEQ ID NO: 127 - protein, 16 aa, Artificial Sequence
  source: /note="Description of Artificial Sequence: Synthetic peptide"
  VARIANT (2)..(2): /replace="Arg" or "Glu"
  SITE (1)..(16): /note="Variant residues given in the sequence have no preference with respect to those in the annotations for variant positions"
LAQETFSDLWKLLPEN

SEQ ID NO: 128 - protein, 16 aa, Artificial Sequence
  source: /note="Description of Artificial Sequence: Synthetic peptide"
  VARIANT (3)..(3): /replace="Arg"
  SITE (1)..(16): /note="Variant residues given in the sequence have no preference with respect to those in the annotations for variant positions"
LSAETFSDLWKLLPEN

SEQ ID NO: 129 - protein, 16 aa, Artificial Sequence
  source: /note="Description of Artificial Sequence: Synthetic peptide"
LSQRTFSDLWKLLPEN

SEQ ID NO: 130 - protein, 16 aa, Artificial Sequence
  source: /note="Description of Artificial Sequence: Synthetic peptide"
  VARIANT (5)..(5): /replace="Arg" or "Glu"
  SITE (1)..(16): /note="Variant residues given in the sequence have no preference with respect to those in the annotations for variant positions"
LSQEAFSDLWKLLPEN

SEQ ID NO: 131 - protein, 16 aa, Artificial Sequence
  source: /note="Description of Artificial Sequence: Synthetic peptide"
  VARIANT (8)..(8): /replace="Glu"
  SITE (1)..(16): /note="Variant residues given in the sequence have no preference with respect to those in the annotations for variant positions"
LSQETFSRLWKLLPEN

SEQ ID NO: 132 - protein, 16 aa, Artificial Sequence
  source: /note="Description of Artificial Sequence: Synthetic peptide"
  VARIANT (9)..(9): /replace="Glu"
  SITE (1)..(16): /note="Variant residues given in the sequence have no preference with respect to those in the annotations for variant positions"
LSQETFSDAWKLLPEN

SEQ ID NO: 133 - protein, 16 aa, Artificial Sequence
  source: /note="Description of Artificial Sequence: Synthetic peptide"
  VARIANT (11)..(11): /replace="Arg"
  SITE (1)..(16): /note="Variant residues given in the sequence have no preference with respect to those in the annotations for variant positions"
LSQETFSDLWALLPEN

SEQ ID NO: 134 - protein, 16 aa, Artificial Sequence
  source: /note="Description of Artificial Sequence: Synthetic peptide"
  VARIANT (12)..(12): /replace="Arg" or "Glu"
  SITE (1)..(16): /note="Variant residues given in the sequence have no preference with respect to those in the annotations for variant positions"
LSQETFSDLWKALPEN

SEQ ID NO: 135 - protein, 16 aa, Artificial Sequence
  source: /note="Description of Artificial Sequence: Synthetic peptide"
  VARIANT (15)..(15): /replace="Arg"
  SITE (1)..(16): /note="Variant residues given in the sequence have no preference with respect to those in the annotations for variant positions"
LSQETFSDLWKLLPAN

SEQ ID NO: 136 - protein, 16 aa, Artificial Sequence
  source: /note="Description of Artificial Sequence: Synthetic peptide"
  VARIANT (16)..(16): /replace="Glu"
  SITE (1)..(16): /note="Variant residues given in the sequence have no preference with respect to those in the annotations for variant positions"
LSQETFSDLWKLLPEA

SEQ ID NO: 137 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWEQXXSAA

SEQ ID NO: 138 - protein, 14 aa, Artificial Sequence
  source: /note="Description of Artificial Sequence: Synthetic peptide"
  VARIANT (10)..(10): /replace="Ile" or "Leu" or "norleucine"
  SITE (1)..(14): /note="Variant residues given in the sequence have no preference with respect to those in the annotations for variant positions"
LTFEEYWAQFTSAA

SEQ ID NO: 139 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWAQXXAAA

SEQ ID NO: 140 - protein, 14 aa, Artificial Sequence
  source: /note="Description of Artificial Sequence: Synthetic peptide"
  MOD_RES (4)..(4): stapling amino acid
  MOD_RES (10)..(10): cyclobutylalanine
  MOD_RES (11)..(11): stapling amino acid
LTFXEYWEQXXEAA

SEQ ID NO: 141-143 - skipped (3 entries)

SEQ ID NO: 144 - protein, 14 aa, Artificial Sequence
  source: /note="Description of Artificial Sequence: Synthetic peptide"
  VARIANT (1)..(1): /replace="conservative amino acid substitution of Leu"
  VARIANT (2)..(2): /replace="conservative amino acid substitution of Thr"
  VARIANT (3)..(3): /replace="conservative amino acid substitution of Phe"
  MOD_RES (4)..(4): stapling amino acid
  VARIANT (5)..(5): /replace="conservative amino acid substitution of Glu"
  VARIANT (6)..(6): /replace="conservative amino acid substitution of Tyr"
  VARIANT (7)..(7): /replace="conservative amino acid substitution of Trp"
  VARIANT (8)..(8): /replace="conservative amino acid substitution of Ala"
  VARIANT (9)..(9): /replace="conservative amino acid substitution of Gln"
  VARIANT (10)..(10): /replace="charged amino acid" or "hydrophobic amino acid less hydrophobic than alanine" or "hydrophilic amino acid"
  MOD_RES (11)..(11): stapling amino acid
  VARIANT (12)..(12): /replace="conservative amino acid substitution of Ser"
  VARIANT (13)..(13): /replace="conservative amino acid substitution of Ala"
  VARIANT (14)..(14): /replace="conservative amino acid substitution of Ala"
  SITE (1)..(14): /note="Variant residues given in the sequence have no preference with respect to those in the annotations for variant positions"
LTFXEYWAQAXSAA

SEQ ID NO: 145 - protein, 14 aa, Artificial Sequence
  source: /note="Description of Artificial Sequence: Synthetic peptide"
  VARIANT (1)..(1): /replace="conservative amino acid substitution of Leu"
  VARIANT (2)..(2): /replace="conservative amino acid substitution of Thr"
  VARIANT (3)..(3): /replace="charged amino acid"
  MOD_RES (4)..(4): stapling amino acid
  VARIANT (5)..(5): /replace="conservative amino acid substitution of Glu"
  VARIANT (6)..(6): /replace="conservative amino acid substitution of Tyr"
  VARIANT (7)..(7): /replace="conservative amino acid substitution of Trp"
  VARIANT (8)..(8): /replace="conservative amino acid substitution of Ala"
  VARIANT (9)..(9): /replace="conservative amino acid substitution of Gln"
  VARIANT (10)..(10): /replace="cyclobutyl alanine"
  MOD_RES (11)..(11): stapling amino acid
  VARIANT (12)..(12): /replace="conservative amino acid substitution of Ser"
  VARIANT (13)..(13): /replace="conservative amino acid substitution of Ala"
  VARIANT (14)..(14): /replace="conservative amino acid substitution of Ala"
  SITE (1)..(14): /note="Variant residues given in the sequence have no preference with respect to those in the annotations for variant positions"
LTAXEYWAQLXSAA

SEQ ID NO: 146 - protein, 14 aa, Artificial Sequence
  source: /note="Description of Artificial Sequence: Synthetic peptide"
  VARIANT (1)..(1): /replace="conservative amino acid substitution of Leu"
  VARIANT (2)..(2): /replace="conservative amino acid substitution of Thr"
  VARIANT (3)..(3): /replace="conservative amino acid substitution of Phe"
  MOD_RES (4)..(4): stapling amino acid
  VARIANT (5)..(5): /replace="conservative amino acid substitution of Glu"
  VARIANT (6)..(6): /replace="conservative amino acid substitution of Tyr"
  VARIANT (7)..(7): /replace="charged amino acid" or "aromatic amino acid other than W"
  VARIANT (8)..(8): /replace="conservative amino acid substitution of Ala"
  VARIANT (9)..(9): /replace="conservative amino acid substitution of Gln"
  VARIANT (10)..(10): /replace="cyclobutyl alanine"
  MOD_RES (11)..(11): stapling amino acid
  VARIANT (12)..(12): /replace="conservative amino acid substitution of Ser"
  VARIANT (13)..(13): /replace="conservative amino acid substitution of Ala"
  VARIANT (14)..(14): /replace="conservative amino acid substitution of Ala"
  SITE (1)..(14): /note="Variant residues given in the sequence have no preference with respect to those in the annotations for variant positions"
LTFXEYAAQLXSAA

SEQ ID NO: 147 - protein, 16 aa, Artificial Sequence
  source: /note="Description of Artificial Sequence: Synthetic peptide"
  VARIANT (1)..(1): /replace="conservative amino acid substitution of Leu" or "Gln" or "conservative amino acid substitution of Gln" or " "
  VARIANT (2)..(2): /replace="conservative amino acid substitution of Ser" or " "
  VARIANT (3)..(3): /replace="conservative amino acid substitution of Gln" or " "
  VARIANT (4)..(4): /replace="conservative amino acid substitution of Glu" or "Gln" or "conservative amino acid substitution of Gln" or "Leu" or "Ala"
  VARIANT (5)..(5): /replace="conservative amino acid substitution of Thr" or "Ala"
  VARIANT (6)..(6): /replace="conservative amino acid substitution of Phe" or "Ala" or "Arg" or "Glu" or " "
  VARIANT (7)..(7): /replace="Glu"
  VARIANT (8)..(8): /replace="conservative amino acid substitution of Asp" or "Asn" or "conservative amino acid substitution of Asn" or "Glu" or "Ala"
  VARIANT (9)..(9): /replace="conservative amino acid substitution of Leu" or "Tyr" or "Arg"
  VARIANT (10)..(10): /replace="conservative amino acid substitution of Trp" or "Ala" or "Arg" or "Glu"
  VARIANT (11)..(11): /replace="conservative amino acid substitution of Lys" or "Arg" or"conservative amino acid substitution of Arg" or "Ala" or "Glu"
  VARIANT (12)..(12): /replace="conservative amino acid substitution of Leu" or "Gln" or "Lys" or "Ala"
  VARIANT (13)..(13): /replace="charged amino acid" or "hydrophobic amino acid less hydrophobic than alanine" or "hydrophilic amino acid" or "Arg" or "Glu" or "E(OMe)" or "Met(O)" or "homoglutamic acid" or "5-fluoronorvaline" or "Gly" or "His" or "Lys" or "Asn" or
  VARIANT (13)..(13): /replace="Gln" or "Ser" or "Thr" or "Val" or "Trp" or "Tyr" or "Prp" or "Cys"
  VARIANT (14)..(14): /replace="Thr"
  VARIANT (15)..(15): /replace="conservative amino acid substitution of Glu" or "Gln" or "conservative amino acid substitution of Gln" or "Ser" or "Ala"
  VARIANT (16)..(16): /replace="conservative amino acid substitution of Asn" or "Ala" or " "
  SITE (1)..(16): /note="Variant residues given in the sequence have no preference with respect to those in the annotations for variant positions"
LSQETFSDLWKLAPEN

SEQ ID NO: 148 - protein, 16 aa, Artificial Sequence
  source: /note="Description of Artificial Sequence: Synthetic peptide"
  VARIANT (1)..(1): /replace="conservative amino acid substitution of Leu" or "Gln" or "conservative amino acid substitution of Gln"
  VARIANT (2)..(2): /replace="conservative amino acid substitution of Ser"
  VARIANT (3)..(3): /replace="conservative amino acid substitution of Gln"
  VARIANT (4)..(4): /replace="conservative amino acid substitution of Glu" or "Gln" or "conservative amino acid substitution of Gln"
  VARIANT (5)..(5): /replace="conservative amino acid substitution of Thr"
  VARIANT (6)..(6): /replace="charged amino acid"
  MOD_RES (7)..(7): stapling amino acid
  VARIANT (8)..(8): /replace="conservative amino acid substitution of Asp" or "Asn" or "conservative amino acid substitution of Asn"
  VARIANT (9)..(9): /replace="conservative amino acid substitution of Leu"
  VARIANT (10)..(10): /replace="conservative amino acid substitution of Trp"
  VARIANT (11)..(11): /replace="conservative amino acid substitution of Lys" or "Arg" or "conservative amino acid substitution of Arg"
  VARIANT (12)..(12): /replace="conservative amino acid substitution of Leu"
  VARIANT (13)..(13): /replace="conservative amino acid substitution of Leu"
  MOD_RES (14)..(14): stapling amino acid
  VARIANT (15)..(15): /replace="conservative amino acid substitution of Glu" or "Gln" or "conservative amino acid substitution of Gln"
  VARIANT (16)..(16): /replace="conservative amino acid substitution of Asn"
  SITE (1)..(16): /note="Variant residues given in the sequence have no preference with respect to those in the annotations for variant positions"
LSQETAXDLWKLLXEN

SEQ ID NO: 149 - protein, 16 aa, Artificial Sequence
  source: /note="Description of Artificial Sequence: Synthetic peptide"
  VARIANT (1)..(1): /replace="conservative amino acid substitution of Leu" or "Gln" or "conservative amino acid substitution of Gln"
  VARIANT (2)..(2): /replace="conservative amino acid substitution of Ser"
  VARIANT (3)..(3): /replace="conservative amino acid substitution of Gln"
  VARIANT (4)..(4): /replace="conservative amino acid substitution of Glu" or "Gln" or "conservative amino acid substitution of Gln"
  VARIANT (5)..(5): /replace="conservative amino acid substitution of Thr"
  VARIANT (6)..(6): /replace="conservative amino acid substitution of Phe"
  MOD_RES (7)..(7): stapling amino acid
  VARIANT (8)..(8): /replace="conservative amino acid substitution of Asp" or "Asn" or "conservative amino acid substitution of Asn"
  VARIANT (9)..(9): /replace="conservative amino acid substitution of Leu"
  VARIANT (10)..(10): /replace="charged amino acid" or "aromatic amino acid other than W"
  VARIANT (11)..(11): /replace="conservative amino acid substitution of Lys" or "Arg" or "conservative amino acid substitution of Arg"
  VARIANT (12)..(12): /replace="conservative amino acid substitution of Leu"
  VARIANT (13)..(13): /replace="conservative amino acid substitution of Leu"
  MOD_RES (14)..(14): stapling amino acid
  VARIANT (15)..(15): /replace="conservative amino acid substitution of Glu" or "Gln" or "conservative amino acid substitution of Gln"
  VARIANT (16)..(16): /replace="conservative amino acid substitution of Asn"
  SITE (1)..(16): /note="Variant residues given in the sequence have no preference with respect to those in the annotations for variant positions"
LSQETFXDLAKLLXEN

SEQ ID NO: 150 - protein, 6 aa, Artificial Sequence
  source: /note="Description of Artificial Sequence: Synthetic peptide"
ASQETF

SEQ ID NO: 151 - protein, 6 aa, Artificial Sequence
  source: /note="Description of Artificial Sequence: Synthetic peptide"
DLWKLL

SEQ ID NO: 152 - protein, 6 aa, Artificial Sequence
  source: /note="Description of Artificial Sequence: Synthetic peptide"
LAQETF

SEQ ID NO: 153 - protein, 6 aa, Artificial Sequence
  source: /note="Description of Artificial Sequence: Synthetic peptide"
LSAETF

SEQ ID NO: 154 - protein, 6 aa, Artificial Sequence
  source: /note="Description of Artificial Sequence: Synthetic peptide"
LSQATF

SEQ ID NO: 155 - protein, 6 aa, Artificial Sequence
  source: /note="Description of Artificial Sequence: Synthetic peptide"
LSQEAF

SEQ ID NO: 156 - protein, 6 aa, Artificial Sequence
  source: /note="Description of Artificial Sequence: Synthetic peptide"
LSQETA

SEQ ID NO: 157 - protein, 6 aa, Artificial Sequence
  source: /note="Description of Artificial Sequence: Synthetic peptide"
LSQETF

SEQ ID NO: 158 - protein, 6 aa, Artificial Sequence
  source: /note="Description of Artificial Sequence: Synthetic peptide"
ALWKLL

SEQ ID NO: 159 - protein, 6 aa, Artificial Sequence
  source: /note="Description of Artificial Sequence: Synthetic peptide"
DAWKLL

SEQ ID NO: 160 - protein, 6 aa, Artificial Sequence
  source: /note="Description of Artificial Sequence: Synthetic peptide"
DLAKLL

SEQ ID NO: 161 - protein, 6 aa, Artificial Sequence
  source: /note="Description of Artificial Sequence: Synthetic peptide"
DLWALL

SEQ ID NO: 162 - protein, 6 aa, Artificial Sequence
  source: /note="Description of Artificial Sequence: Synthetic peptide"
RSQETF

SEQ ID NO: 163 - protein, 6 aa, Artificial Sequence
  source: /note="Description of Artificial Sequence: Synthetic peptide"
LRQETF

SEQ ID NO: 164 - protein, 6 aa, Artificial Sequence
  source: /note="Description of Artificial Sequence: Synthetic peptide"
LSRETF

SEQ ID NO: 165 - protein, 6 aa, Artificial Sequence
  source: /note="Description of Artificial Sequence: Synthetic peptide"
LSQRTF

SEQ ID NO: 166 - protein, 6 aa, Artificial Sequence
  source: /note="Description of Artificial Sequence: Synthetic peptide"
LSQERF

SEQ ID NO: 167 - protein, 6 aa, Artificial Sequence
  source: /note="Description of Artificial Sequence: Synthetic peptide"
LSQETR

SEQ ID NO: 168 - protein, 6 aa, Artificial Sequence
  source: /note="Description of Artificial Sequence: Synthetic peptide"
RLWKLL

SEQ ID NO: 169 - protein, 6 aa, Artificial Sequence
  source: /note="Description of Artificial Sequence: Synthetic peptide"
DRWKLL

SEQ ID NO: 170 - protein, 6 aa, Artificial Sequence
  source: /note="Description of Artificial Sequence: Synthetic peptide"
DLRKLL

SEQ ID NO: 171 - protein, 6 aa, Artificial Sequence
  source: /note="Description of Artificial Sequence: Synthetic peptide"
DLWRLL

SEQ ID NO: 172 - protein, 6 aa, Artificial Sequence
  source: /note="Description of Artificial Sequence: Synthetic peptide"
DLWKRL

SEQ ID NO: 173 - protein, 6 aa, Artificial Sequence
  source: /note="Description of Artificial Sequence: Synthetic peptide"
DLWKLR

SEQ ID NO: 174 - protein, 6 aa, Artificial Sequence
  source: /note="Description of Artificial Sequence: Synthetic peptide"
ESQETF

SEQ ID NO: 175 - protein, 6 aa, Artificial Sequence
  source: /note="Description of Artificial Sequence: Synthetic peptide"
LEQETF

SEQ ID NO: 176 - protein, 6 aa, Artificial Sequence
  source: /note="Description of Artificial Sequence: Synthetic peptide"
LSEETF

SEQ ID NO: 177 - protein, 6 aa, Artificial Sequence
  source: /note="Description of Artificial Sequence: Synthetic peptide"
LSQEEF

SEQ ID NO: 178 - protein, 6 aa, Artificial Sequence
  source: /note="Description of Artificial Sequence: Synthetic peptide"
LSQETE

SEQ ID NO: 179 - protein, 6 aa, Artificial Sequence
  source: /note="Description of Artificial Sequence: Synthetic peptide"
ELWKLL

SEQ ID NO: 180 - protein, 6 aa, Artificial Sequence
  source: /note="Description of Artificial Sequence: Synthetic peptide"
DEWKLL

SEQ ID NO: 181 - protein, 6 aa, Artificial Sequence
  source: /note="Description of Artificial Sequence: Synthetic peptide"
DLEKLL

SEQ ID NO: 182 - protein, 6 aa, Artificial Sequence
  source: /note="Description of Artificial Sequence: Synthetic peptide"
DLWELL

SEQ ID NO: 183 - protein, 6 aa, Artificial Sequence
  source: /note="Description of Artificial Sequence: Synthetic peptide"
DLWKEL

SEQ ID NO: 184 - protein, 6 aa, Artificial Sequence
  source: /note="Description of Artificial Sequence: Synthetic peptide"
DLWKLE

SEQ ID NO: 185 - protein, 6 aa, Artificial Sequence
  source: /note="Description of Artificial Sequence: Synthetic peptide"
DLWELR

SEQ ID NO: 186 - protein, 6 aa, Artificial Sequence
  source: /note="Description of Artificial Sequence: Synthetic peptide"
DLWELA

SEQ ID NO: 187 - protein, 6 aa, Artificial Sequence
  source: /note="Description of Artificial Sequence: Synthetic peptide"
DLWELE

SEQ ID NO: 188 - protein, 6 aa, Artificial Sequence
  source: /note="Description of Artificial Sequence: Synthetic peptide"
DRWKLE

SEQ ID NO: 189 - protein, 6 aa, Artificial Sequence
  source: /note="Description of Artificial Sequence: Synthetic peptide"
DRWKLR

SEQ ID NO: 190 - protein, 6 aa, Artificial Sequence
  source: /note="Description of Artificial Sequence: Synthetic peptide"
DRWKLA

SEQ ID NO: 191 - protein, 6 aa, Artificial Sequence
  source: /note="Description of Artificial Sequence: Synthetic peptide"
EYWAQD

SEQ ID NO: 192 - protein, 6 aa, Artificial Sequence
  source: /note="Description of Artificial Sequence: Synthetic peptide"
EYWAQA

SEQ ID NO: 193 - protein, 6 aa, Artificial Sequence
  source: /note="Description of Artificial Sequence: Synthetic peptide"
EYWAQR

SEQ ID NO: 194 - protein, 6 aa, Artificial Sequence
  source: /note="Description of Artificial Sequence: Synthetic peptide"
EYWAQE

SEQ ID NO: 195 - protein, 6 aa, Artificial Sequence
  source: /note="Description of Artificial Sequence: Synthetic peptide"
EYWAQF

SEQ ID NO: 196 - protein, 6 aa, Artificial Sequence
  source: /note="Description of Artificial Sequence: Synthetic peptide"
EYWAQG

SEQ ID NO: 197 - protein, 6 aa, Artificial Sequence
  source: /note="Description of Artificial Sequence: Synthetic peptide"
EYWAQH

SEQ ID NO: 198 - protein, 6 aa, Artificial Sequence
  source: /note="Description of Artificial Sequence: Synthetic peptide"
EYWAQI

SEQ ID NO: 199 - protein, 6 aa, Artificial Sequence
  source: /note="Description of Artificial Sequence: Synthetic peptide"
EYWAQK

SEQ ID NO: 200 - protein, 6 aa, Artificial Sequence
  source: /note="Description of Artificial Sequence: Synthetic peptide"
EYWAQL

SEQ ID NO: 201 - protein, 6 aa, Artificial Sequence
  source: /note="Description of Artificial Sequence: Synthetic peptide"
  MOD_RES (6)..(6): norleucine
EYWAQX

SEQ ID NO: 202 - protein, 6 aa, Artificial Sequence
  source: /note="Description of Artificial Sequence: Synthetic peptide"
EYWAQN

SEQ ID NO: 203 - protein, 6 aa, Artificial Sequence
  source: /note="Description of Artificial Sequence: Synthetic peptide"
EYWAQQ

SEQ ID NO: 204 - protein, 6 aa, Artificial Sequence
  source: /note="Description of Artificial Sequence: Synthetic peptide"
EYWAQS

SEQ ID NO: 205 - protein, 6 aa, Artificial Sequence
  source: /note="Description of Artificial Sequence: Synthetic peptide"
EYWAQT

SEQ ID NO: 206 - protein, 6 aa, Artificial Sequence
  source: /note="Description of Artificial Sequence: Synthetic peptide"
EYWAQV

SEQ ID NO: 207 - protein, 6 aa, Artificial Sequence
  source: /note="Description of Artificial Sequence: Synthetic peptide"
EYWAQW

SEQ ID NO: 208 - protein, 6 aa, Artificial Sequence
  source: /note="Description of Artificial Sequence: Synthetic peptide"
EYWAQY

SEQ ID NO: 209 - protein, 6 aa, Artificial Sequence
  source: /note="Description of Artificial Sequence: Synthetic peptide"
EYWAQP

SEQ ID NO: 210 - protein, 6 aa, Artificial Sequence
  source: /note="Description of Artificial Sequence: Synthetic peptide"
EYWAQC

SEQ ID NO: 211 - protein, 6 aa, Artificial Sequence
  source: /note="Description of Artificial Sequence: Synthetic peptide"
  MOD_RES (6)..(6): cyclobutylalanine
ERWAQX

SEQ ID NO: 212 - protein, 6 aa, Artificial Sequence
  source: /note="Description of Artificial Sequence: Synthetic peptide"
  MOD_RES (6)..(6): cyclobutylalanine
EYWEQX

SEQ ID NO: 213 - protein, 6 aa, Artificial Sequence
  source: /note="Description of Artificial Sequence: Synthetic peptide"
  MOD_RES (6)..(6): homoglutamic acid
EYWAQX

SEQ ID NO: 214 - protein, 6 aa, Artificial Sequence
  source: /note="Description of Artificial Sequence: Synthetic peptide"
  MOD_RES (6)..(6): cyclobutylalanine
EYWAQX

SEQ ID NO: 215 - protein, 6 aa, Artificial Sequence
  source: /note="Description of Artificial Sequence: Synthetic peptide"
  MOD_RES (6)..(6): cyclobutylalanine
EYWEQX

SEQ ID NO: 216 - protein, 6 aa, Artificial Sequence
  source: /note="Description of Artificial Sequence: Synthetic peptide"
  MOD_RES (6)..(6): cyclobutylalanine
ERWAQX

SEQ ID NO: 217 - skipped

SEQ ID NO: 218 - protein, 6 aa, Artificial Sequence
  source: /note="Description of Artificial Sequence: Synthetic peptide"
  MOD_RES (6)..(6): 5-fluoronorvaline
EYWAQX

SEQ ID NO: 219 - protein, 6 aa, Artificial Sequence
  source: /note="Description of Artificial Sequence: Synthetic peptide"
EYWRQE

SEQ ID NO: 220 - protein, 6 aa, Artificial Sequence
  source: /note="Description of Artificial Sequence: Synthetic peptide"
EYWLQE

SEQ ID NO: 221 - protein, 6 aa, Artificial Sequence
  source: /note="Description of Artificial Sequence: Synthetic peptide"
LYWAQE

SEQ ID NO: 222 - protein, 6 aa, Artificial Sequence
  source: /note="Description of Artificial Sequence: Synthetic peptide"
EYWFQE

SEQ ID NO: 223 - protein, 6 aa, Artificial Sequence
  source: /note="Description of Artificial Sequence: Synthetic peptide"
FYWAQE

SEQ ID NO: 224 - protein, 6 aa, Artificial Sequence
  source: /note="Description of Artificial Sequence: Synthetic peptide"
ERWAQE

SEQ ID NO: 225 - protein, 6 aa, Artificial Sequence
  source: /note="Description of Artificial Sequence: Synthetic peptide"
QSQQTF

SEQ ID NO: 226 - protein, 6 aa, Artificial Sequence
  source: /note="Description of Artificial Sequence: Synthetic peptide"
NLWRLE

SEQ ID NO: 227 - protein, 6 aa, Artificial Sequence
  source: /note="Description of Artificial Sequence: Synthetic peptide"
  MOD_RES (6)..(6): methionine-sulfoxide
EYWAQX

SEQ ID NO: 228 - protein, 6 aa, Artificial Sequence
  source: /note="Description of Artificial Sequence: Synthetic peptide"
  MOD_RES (6)..(6): E(OMe)
EYWAQX

SEQ ID NO: 229 - DNA, 23 bp, Artificial Sequence
  source: /note="Description of Artificial Sequence: Synthetic primer"
agccctctctttgatatgctaag

SEQ ID NO: 230 - DNA, 19 bp, Artificial Sequence
  source: /note="Description of Artificial Sequence: Synthetic primer"
tgggtctttcacggagaag

SEQ ID NO: 231 - DNA, 28 bp, Artificial Sequence
  source: /note="Description of Artificial Sequence: Synthetic primer"
aagccctctcgcggatatgctaagaaag

SEQ ID NO: 232 - DNA, 26 bp, Artificial Sequence
  source: /note="Description of Artificial Sequence: Synthetic primer"
gggtctttcacggagaagctctgacg

SEQ ID NO: 233 - protein, 90 aa, Artificial Sequence
  source: /note="Description of Artificial Sequence: Synthetic polypeptide"
LPGEGTQVHPRAPLLQILKVAGAQEEVFTVKEVMHYLGQYIMMKQLYDKQRQHIVHCHDDPLGELLEVGSFSVKNPSPLYEMLKRNLVIL

SEQ ID NO: 234 - protein, 6 aa, Artificial Sequence
  source: /note="Description of Artificial Sequence: Synthetic peptide"
DLWKAL

SEQ ID NO: 235 - protein, 6 aa, Artificial Sequence
  source: /note="Description of Artificial Sequence: Synthetic peptide"
DLWKLA

SEQ ID NO: 236 - protein, 6 aa, Artificial Sequence
  source: /note="Description of Artificial Sequence: Synthetic 6xHis tag"
HHHHHH

SEQ ID NO: 237 - protein, 6 aa, Artificial Sequence
  source: /note="Description of Artificial Sequence: Synthetic peptide"
  MOD_RES (6)..(6): E(OMe)
NLWRLX

SEQ ID NO: 238 - protein, 7 aa, Artificial Sequence
  source: /note="Description of Artificial Sequence: Synthetic peptide"
  MOD_RES (4)..(4): stapling amino acid
AQEXSAA

SEQ ID NO: 239 - protein, 7 aa, Artificial Sequence
  source: /note="Description of Artificial Sequence: Synthetic peptide"
AQETAAA

SEQ ID NO: 240 - protein, 16 aa, Artificial Sequence
  source: /note="Description of Artificial Sequence: Synthetic peptide"
  VARIANT (1)..(1): /replace="conservative amino acid substitution of Leu" or "Gln" or "conservative amino acid substitution of Gln"
  VARIANT (2)..(2): /replace="conservative amino acid substitution of Ser"
  VARIANT (3)..(3): /replace="conservative amino acid substitution of Gln"
  VARIANT (4)..(4): /replace="conservative amino acid substitution of Glu" or "Gln" or "conservative amino acid substitution of Gln"
  VARIANT (5)..(5): /replace="conservative amino acid substitution of Thr"
  VARIANT (6)..(6): /replace="conservative amino acid substitution of Phe"
  MOD_RES (7)..(7): stapling amino acid
  VARIANT (8)..(8): /replace="conservative amino acid substitution of Asp" or "Asn" or "conservative amino acid substitution of Asn"
  VARIANT (9)..(9): /replace="conservative amino acid substitution of Leu"
  VARIANT (10)..(10): /replace="conservative amino acid substitution of Trp"
  VARIANT (11)..(11): /replace="conservative amino acid substitution of Lys" or "Arg" or "conservative amino acid substitution of Arg"
  VARIANT (12)..(12): /replace="conservative amino acid substitution of Leu"
  VARIANT (13)..(13): /replace="charged amino acid" or "hydrophobic amino acid less hydrophobic than alanine" or "hydrophilic amino acid"
  MOD_RES (14)..(14): stapling amino acid
  VARIANT (15)..(15): /replace="conservative amino acid substitution of Glu" or "Gln" or "conservative amino acid substitution of Gln"
  VARIANT (16)..(16): /replace="conservative amino acid substitution of Asn"
  SITE (1)..(16): /note="Variant residues given in the sequence have no preference with respect to those in the annotations for variant positions"
LSQETFXDLWKLAXEN

SEQ ID NO: 241 - protein, 14 aa, Artificial Sequence
  source: /note="Description of Artificial Sequence: Synthetic peptide"
  VARIANT (1)..(1): /replace="conservative amino acid substitution of Leu"
  VARIANT (2)..(2): /replace="conservative amino acid substitution of Thr"
  VARIANT (3)..(3): /replace="conservative amino acid substitution of Phe"
  MOD_RES (4)..(4): stapling amino acid
  VARIANT (5)..(5): /replace="conservative amino acid substitution of Glu"
  VARIANT (6)..(6): /replace="conservative amino acid substitution of Tyr"
  VARIANT (7)..(7): /replace="conservative amino acid substitution of Trp"
  VARIANT (8)..(8): /replace="conservative amino acid substitution of Ala"
  VARIANT (9)..(9): /replace="conservative amino acid substitution of Gln"
  MOD_RES (10)..(10): Any amino acid except phenylalanine, isoleucine, norleucine, and leucine
  MOD_RES (11)..(11): stapling amino acid
  VARIANT (12)..(12): /replace="conservative amino acid substitution of Ser"
  VARIANT (13)..(13): /replace="conservative amino acid substitution of Ala"
  VARIANT (14)..(14): /replace="conservative amino acid substitution of Ala"
  SITE (1)..(14): /note="Variant residues given in the sequence have no preference with respect to those in the annotations for variant positions"
LTFXEYWAQXXSAA

SEQ ID NO: 242 - protein, 16 aa, Artificial Sequence
  source: /note="Description of Artificial Sequence: Synthetic peptide"
  VARIANT (1)..(1): /replace="conservative amino acid substitution of Leu" or "Gln" or "conservative amino acid substitution of Gln"
  VARIANT (2)..(2): /replace="conservative amino acid substitution of Ser"
  VARIANT (3)..(3): /replace="conservative amino acid substitution of Gln"
  VARIANT (4)..(4): /replace="conservative amino acid substitution of Glu" or "Gln" or "conservative amino acid substitution of Gln"
  VARIANT (5)..(5): /replace="conservative amino acid substitution of Thr"
  VARIANT (6)..(6): /replace="conservative amino acid substitution of Phe"
  MOD_RES (7)..(7): stapling amino acid
  VARIANT (8)..(8): /replace="conservative amino acid substitution of Asp" or "Asn" or "conservative amino acid substitution of Asn"
  VARIANT (9)..(9): /replace="conservative amino acid substitution of Leu"
  VARIANT (10)..(10): /replace="conservative amino acid substitution of Trp"
  VARIANT (11)..(11): /replace="conservative amino acid substitution of Lys" or "Arg" or "conservative amino acid substitution of Arg"
  VARIANT (12)..(12): /replace="conservative amino acid substitution of Leu"
  MOD_RES (13)..(13): Any amino acid except phenylalanine, isoleucine, norleucine, and leucine
  MOD_RES (14)..(14): stapling amino acid
  VARIANT (15)..(15): /replace="conservative amino acid substitution of Glu" or "Gln" or "conservative amino acid substitution of Gln"
  VARIANT (16)..(16): /replace="conservative amino acid substitution of Asn"
  SITE (1)..(16): /note="Variant residues given in the sequence have no preference with respect to those in the annotations for variant positions"
LSQETFXDLWKLXXEN